(19) United States
(12) Patent Application Publication
(10) Pub. No.: US 2023/0082376 A1
(43) Pub. Date: Mar. 16, 2023

(54) DIRECTED GAS FLOW ACCESSORY FOR PROVIDING GASES TO AND VENTING GASES FROM A PATIENT

(71) Applicant: FISHER & PAYKEL HEALTHCARE LIMITED, Auckland (NZ)

(72) Inventors: Zach Jonathan WARNER, Auckland (NZ); Jonathan Stuart MCFEDRIES, Auckland (NZ); Lucila San Jose DE JESUS, Auckland (NZ); German KLINK, Auckland (NZ); Simon REVELLY, Auckland (NZ); Joshua Robert LEE, Auckland (NZ); Katie-Ann Jane BUCKELS, Auckland (NZ); Rory Alexander MONRO, Auckland (NZ); Jemma Tamsin SOMERVILLE, Auckland (NZ); Bernard Tsz Lun IP, Auckland (NZ); Zane Paul GELL, Auckland (NZ); Danielle Elizabeth EVANS, Auckland (NZ); Eu-Lee TEH, Auckland (NZ); Monika BAUMANN, Auckland (NZ); Jasmine Emerald Michelle LOUIE, Auckland (NZ); Abigail Sharmini Rajen ARULANDU, Auckland (NZ); Jose FLORES VAZQUEZ, Auckland (NZ); Lotte Gertrudis Theodora VAN DEN HEUIJ, Auckland (NZ); James Michael GILBERT, Auckland (NZ)

(21) Appl. No.: 17/904,099

(22) PCT Filed: Feb. 12, 2021
(86) PCT No.: PCT/IB2021/051155

§ 371 Date: Aug. 11, 2022

Related U.S. Application Data
(63) Continuation of application No. 62/976,993, filed on Feb. 14, 2020.

(51) Int. Cl. A61B 1/00; A61B 1/015

(52) U.S. Cl. CPC A61B 1/00131; A61B 1/015

(57) ABSTRACT

Disclosed herein is a cannula and/or medical instrument accessory configured for providing localized insufflation or venting of gases with respect to a surgical cavity of a patient (such as the pneumoperitoneum) and allowing insertion of medical instruments into the surgical cavity through the cannula. A medical instrument accessory such as a cannula and/or medical instrument accessory can be used for localizing insufflation or venting of gas or fluid near operating end of a medical instrument. The medical instrument accessory can comprise a body mountable over at least a portion of a medical instrument shaft, the body having an inner lumen, proximal end and distal end, the distal end comprising an opening, wherein the distal end is arranged in use at or adjacent an operating end of the medical instrument. An outer wall of the medical instrument shaft and lumen can define a gas flow path, wherein as or fluid is released or introduced into the gas flow path at the distal end and adjacent the end of the medical instrument shaft.

### CROSS-REFERENCE TO RELATED APPLICATIONS

The present application claims priority from U.S. patent application no. 62/976,993, filed on 14 Feb. 2020, the content of which is hereby incorporated by reference.

### FIELD OF THE DISCLOSURE

The present disclosure relates, generally, to medical instrument accessories and components of medical instrument accessories, and, in particular, to such accessories configured to direct gases to a patient and/or vent gases from a patient, in particular during a medical procedure.

### BACKGROUND

Various medical procedures require the provision of gases, typically carbon dioxide, to a patient during the procedure. For example, two general categories of medical procedures often require providing gases to a patient, being closed type medical procedures and open type medical procedures.

In closed type medical procedures, an insufflator is arranged to deliver gases to a body cavity of the patient to inflate the body cavity and/or to resist collapse of the body cavity during the procedure. Examples of such medical procedures include laparoscopy and endoscopy, although an insufflator may be used with any other type of medical procedure as required. Endoscopic procedures enable a medical practitioner to visualize a body cavity by inserting an endoscope, or the like, through one or more natural openings, small puncture(s), or incision(s) to generate an image of the body cavity. In laparoscopy procedures, a medical practitioner typically inserts a medical instrument through natural openings, small puncture(s), or incision(s) to perform a medical procedure in the body cavity. In some cases an initial endoscopic procedure may be carried out to assess the body cavity, and then a subsequent laparoscopy carried out to operate on the body cavity. Such procedures are widely used, for example, on the peritoneal cavity, or during a thoracoscopy, colonoscopy, gastroscopy or bronchoscopy.

In open type medical procedures, for example, open surgeries, gases are used to fill a surgical cavity, with excess gases spilling outward from the opening. The gases can also be used to provide a layer of gases over exposed body parts for example, including internal body parts where there is no discernible cavity. For these procedures, rather than serving to inflate a cavity, the gases can be used to prevent or reduce desiccation and infection by covering exposed internal body parts with a layer of heated, humidified, sterile gases.

An apparatus for delivering gases during these medical procedures can include an insufflator arranged to be connected to a remote source of pressurized gases, for example, a gases supply system in a hospital. The apparatus can be operative to control the pressure and/or flow of the gases from the gases source to a level suitable for delivery into the body cavity, usually via a cannula or needle connected to the apparatus and inserted into the body cavity, or via a diffuser arranged to diffuse gases over and into the wound or surgical cavity.

The internal body temperature of a human patient is typically around 37° C. It can be desirable to match the temperature of the gases delivered from the apparatus as closely as possible to the typical human body temperature. It can also be desirable to deliver gases above or below internal body temperature, such as, for example, any degree between 1 to 10° C., at 15° C., or more or less above or below internal body temperature for example, or ranges including any two of the foregoing values. It can also be desirable to deliver gases of a desired fixed or variable humidity and/or a desired fixed or variable temperature. The gases at the desired gas temperature and/or humidity (which may be also referred to herein as standard) can be dry cold gas, dry hot gas, humidified cold gas, or humidified hot gas for example. Further, the gases delivered into the patient's body can be relatively dry, which can cause damage to the body cavity, such as for example cell desiccation, cell death or adhesions. In many cases, a humidifier is operatively coupled to the insufflator. A controller of the apparatus can energize a heater of the humidifier located in the gases flow path to deliver humidification fluid to the gases stream prior to entering the patient's body cavity. The humidification fluid may be water.

The humidified gas can be delivered to the patient via further tubing which may also be heated. The insufflator and humidifier can be located in separate housings that are connected together via suitable tubing and/or electrical connections, or located in a common housing arranged to be connected to a remote gas supply via suitable tubing.

Because of a difference between the temperature of a medical instrument and the temperature within a human body, condensation can occur on surfaces of the instrument when it is introduced to the body. When condensation forms on a viewing surface of a medical instrument, such as a lens of a camera or scope, this causes a fogging effect which impairs visibility through the viewing surface. When condensation forms on the instrument, the condensation can coalesce into water droplets. This can occur directly on the viewing surface or other surfaces which can then migrate to or be deposited on the viewing surface. Accordingly, as used herein condensation and/or fogging means condensation generally and in some instances, specifically with respect to condensation on a viewing surface (i.e. fogging).

When operating a medical instrument within the body, bodily fluids, tissue, or debris may inhibit vision through a viewing surface of the instrument. For example, the viewing surface may be exposed to blood, smoke and/or bone particles which can occlude vision through the surface.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

Condensation occurs when the temperature of a gas falls below the dew point temperature for the level of humidity the gas is carrying, and/or if there are surfaces below the dew-point temperature. The human body is a warm and humid environment, having a temperature of about 37° C. When cold (for example, at or below typical room temperature, and/or below a typical human body temperature) cameras, scopes, or other medical instruments are inserted into this environment, condensation can cause droplets to form on the lens or elsewhere on the scope, which can drip onto the lens area. Similarly, condensation can form droplets on an internal wall of a cannula upper housing and/or shaft and drip down onto the lens area. When such fluid collects on the lens area, this inhibits light transmission through the lens, consequently impairing vision of the operator of the scope. Further, although humidification and heating of insufflation gases can reduce damage to the patient's tissue in a surgical cavity, the humidification and temperature of the gases can exacerbate causing condensation to deposit on and/or about the lens of a scope.

During a medical procedure, various other materials can contact the lens of a scope to inhibit light transmission through the lens. For example, the lens may contact bodily fluids or tissue, or debris or particles created by the procedure, such as surgical smoke. Any such materials or debris on the lens can impede vision, for example, of a surgeon or other medical personnel participating in the medical procedure (for example, surgery). When the lens becomes contaminated by particles, fluid droplets, or the like, it may be necessary to remove the camera and/or the other medical instruments and wipe it (or them) down to remove the contamination. However, removing a medical instrument from the surgical cavity can cause it to cool to below the patient's body temperature. As a result, when the instrument is reinserted to the body, further condensation can form which, again, can inhibit operator vision through the lens. Past approaches to resolve this include pre-warming the medical instruments, and/or using a light or a heating source at the end of the camera to warm the lens. Such interventions typically require additional steps that can negatively impact the workflow and efficiency of the procedure. Furthermore, repetitive heating of instruments, or parts thereof, such as with a heating element adjacent a lens, can affect the structure of the instrument, and/or increase complexity of sterilizing the instrument.

The present disclosure provides examples of a directed gas flow cannula and/or medical instrument accessory, or a medical instrument, configured to direct gas flow relative to an end of the cannula/instrument. In particular, disclosed examples are suitable for localizing insufflation or venting of fluid near, or localizing/directing fluid flow around and/or across, a distal end of a medical instrument. The disclosed cannula and/or medical instrument accessory examples are operable to move fluid and/or debris across and/or away from the end of the cannula/instrument, and/or heat the end. This can inhibit condensation forming droplets at the end of the cannula/instrument, and/or otherwise remove material from the end. Should the instrument be a scope, this can maintain or enhance light transmission through a lens at the end of the scope to enhance the field of vision. Some disclosed examples can direct gases to flow around the scope/medical instrument to affect the environment at or near the lens and/or medical instrument. Other disclosed examples can direct insufflation gases to disperse smoke, condensation, or other unwanted media away from the instrument.

According to one disclosed aspect, there is provided a medical instrument accessory for localizing insufflation or venting of fluid near a distal end of a medical instrument. The medical instrument accessory can include a body which is mountable over at least a portion of a shaft of the medical instrument. The body has an inner lumen, proximal end and distal end, and the distal end comprises an opening. The distal end is configured to be arranged, in use, at or adjacent the distal end of the medical instrument. An outer wall of the medical instrument shaft and the inner lumen define a fluid flow path, such that fluid flows in and/or out of the fluid flow path at or adjacent the distal end of the medical instrument shaft.

The medical instrument accessory described in any of the preceding paragraphs may further comprise one or more of the following features. The body can be elongate. The body can be generally cylindrical. The lumen may be shaped to at least partially surround the shaft of the instrument. The fluid flow path can be at least partly defined by an inner wall of the body and the outer wall of the medical instrument shaft. The body can be configured to attach to the distal end of a cannula or other medical instrument. The body can be at least partially flexible and/or may include an extendible element configured to attach to a distal end of the cannula. The body can be movable between a retracted position and an extended position.

The body can be configured to attach to the medical instrument at the proximal end with an attachment. The attachment can be a sealing attachment. The attachment is configured to create a fluid-tight seal. The proximal end of the body can be in fluid communication with a fluid source and/or vent. The body can have a first portion where the inner lumen has a first diameter substantially the same as the medical instrument outer wall diameter, and a second portion where the inner lumen has a second diameter, wherein the second diameter is greater than the medical instrument outer wall diameter, the body having at least one aperture in fluid communication with the fluid flow path. The diameter of the lumen can transition from the first diameter to the second diameter. The at least one aperture can be located in the transition between the first and the second diameter.

The medical instrument can be a laparoscope and the fluid flow path be configured such that fluid exits or enters the fluid flow path adjacent a lens of the laparoscope. Additionally or alternatively, the fluid flow path can be configured such that fluid exits or enters the fluid flow path parallel to a lens of the laparoscope. The body can define a projection, such as a shoulder or ring portion, arranged at the distal end. The projection can define at least one aperture. The projection may define an inner diameter that is less than an outer diameter of the medical instrument. The medical instrument can be an electrocautery tool. The body can have a length dimensioned to be substantially equivalent to the length of the medical instrument such that the distal end of the body is arranged adjacent the distal end of the medical instrument.

In some cases, a medical instrument accessory, for localizing or directing fluid flow around a distal end of a medical instrument, can comprise a body configured to mount over at least a portion of a shaft of the medical instrument. The body can comprise a lumen with an inner wall, a proximal end, an open distal end, and at least one structure configured to, in use, position the medical instrument shaft in the lumen such that a fluid flow path is defined between the lumen inner wall and the medical instrument shaft. The body may further cause fluid to be directed into or out of the open distal end. The body may also cause fluid to be directed around an end of the medical instrument.

The medical instrument accessory described in any of the preceding paragraphs may further comprise one or more of the following features. The at least one structure can be on the inner wall of the accessory. The body can be configured to fit over at least a portion of the medical instrument shaft. The proximal end of the body can be in fluid communication with a fluid source or vent. The at least one structure can comprise a plurality of structures. The at least one structure can hold the medical instrument shaft substantially concentrically in the lumen.

The at least one structure can comprise one or more sub-structures, such as surfaces, projections or ribs, extending inwardly from the inner wall. The one or more sub-structures can extend substantially along an entire length of the inner wall. The one or more sub-structures can be located at least partially about the open distal end. The one or more sub-structures can be located adjacent the proximal end. The one or more sub-structures can be located adjacent the proximal and distal ends. The at least one structure can comprise one or more protrusions extending inwardly from the inner wall of the lumen. The one or more protrusions can be located at the proximal end, the distal end and/or be intermediate along the length of the lumen. The one or more sub-structures or protrusions can be spaced substantially uniformly around a diameter of the lumen. The one or more sub-structures or protrusions can be spaced non-uniformly. The one or more sub-structures or protrusions may at least partially define a plurality of fluid flow paths, in some embodiments the paths defining different shapes and/or sizes.

The at least one structure can comprise one or more fins extending inwardly from the lumen inner wall. The one or more fins can be arranged in a substantially spiral configuration. The at least one structure can comprise one or more flexible members extending from the open distal end. The at least one structure can comprise a movable tip located at the distal open end, the movable tip having a flexible portion and a solid edge with one or more protrusions extending radially inwardly. The solid edge can be laterally movable and is substantially parallel with the open distal end. The solid edge can be configured to engage with an end of the medical instrument. The lumen can have a cross-sectional shape of a different shape than the medical instrument shaft. The cross-sectional shape of the lumen can be substantially oval shaped. The at least one structure can comprise one or more channels disposed in the inner wall of the lumen. The one or more channels can extend substantially an entire length of the lumen.

In some cases, a medical instrument accessory for directing fluid flow relative to a distal end of a medical instrument, such as around and/or across the distal end, can comprise a body configured to mount over at least a portion of a shaft of the medical instrument to at least partially surround the shaft. The body can comprise a lumen with an inner wall, a proximal end, an open distal end, and a stopping portion which may be arranged at or adjacent the open distal end. The body defines a longitudinal axis between the ends.

The medical instrument accessory described in any of the preceding paragraphs may further comprise one or more of the following features. The stopping portion can be configured to, in use, locate a distal end of the medical instrument to be a predetermined distance away from the open distal end of the body. The medical instrument accessory can further comprise one or more structures, such as ribs or protrusions, disposed on the inner wall of the lumen. The ribs or protrusions can be arranged substantially concentrically around the inner wall of the lumen.

The medical instrument accessory can further comprise at least one deflection structure, such as a shelf, shoulder or ledge, arranged to extend partially across the open distal end to allow receiving fluid flowing through the lumen and deflecting the fluid to flow relative to, for example, transversely to, the longitudinal axis. The, or each, deflection structure may extend radially inwardly, and may be arranged to extend from an edge, side or rim of the open distal end. The deflection structure may include a plurality of deflection surfaces arranged to direct fluid in a respective plurality of streams across the longitudinal axis. The deflection structure may be separate from, and mountable to the accessory, such as to a shaft of the accessory. The deflection structure can extend substantially perpendicularly from the edge of the open distal end, and may be defined by a ring structure. The deflection structure can define a surface area that is a segment of a circle. The stopping portion can be arranged in the lumen to be spaced axially from the deflection structure, or may be arranged at least partially on the deflection structure.

The medical instrument accessory can further comprise a protuberance, such as a flange, extending longitudinally from the open distal end to allow directing fluid flow. The protuberance may extend from an edge or rim of the distal end to partially surround the opening. The protuberance may define a free end and the deflection structure can extend radially inwards from the free end of the protuberance.

The body can include at least one second lumen configured to convey fluid through and out of the body, such as to channel insufflation gas into a surgical cavity. The body can include at least one venting lumen configured to receive and convey fluid through the body, such as to vent fluid from the surgical cavity. The at least one venting lumen can have an inlet arranged in the body. The open distal end can be angled relative to a longitudinal axis of the body.

In some cases, a medical instrument accessory for heating a medical instrument can comprise a body that can be configured to mount over at least a portion of the medical instrument. The body can include a heating device. The heating device may, in use, directly or indirectly heat the medical instrument.

The medical instrument accessory described in any of the preceding paragraphs may further comprise one or more of the following features. The body can have a lumen with an inner wall. The inner wall may be configured to contact a surface of the medical instrument. The inner wall may be configured, in use, to be spaced apart from a surface of the medical instrument. The body may define a length which is less than a length of a shaft of the medical instrument. The heating device can comprise one or more selected from the group consisting of: heating coils, resistive material, flexible PCB, chemical heating, insulated material, and vaporization. The heating device can be powered by one or more of an external unit, an associated cannula, a battery, a tubeset, a tube, and a wireless power transfer. The heating device can provide heating substantially along an entire length of a shaft of the medical instrument. The heating device can be configured to provide graduated heat along a shaft of the medical instrument. The heating device can be configured to provide heat localized to a portion of a shaft of the medical instrument.

In some cases, a medical instrument accessory, for localising or directing fluid flow around a distal end of a medical instrument, can comprise a body configured to mount over at least a portion of a shaft of the medical instrument. The body may have at least one structure configured to, in use, position the medical instrument shaft in a cannula lumen such that a fluid flow path is defined between a wall of the cannula lumen and the medical instrument shaft.

The medical instrument accessory described in any of the preceding paragraphs may further comprise one or more of the following features. The fluid flow path may be configured to direct fluid relative to the distal end of the medical instrument, such as around and/or across the distal end. The at least one structure can comprise a plurality of structures. The at least one structure can be configured to, in use, abut or be adjacent an outer surface of the medical instrument.

In some cases, a medical instrument for use in laparoscopic surgical procedures can comprise a shaft configured to direct fluid flow relative to a shaft of the instrument, such as over or adjacent to a distal end of the shaft.

The medical instrument described in any of the preceding paragraphs may further comprise one or more of the following features. The shaft may have a lumen to direct fluid flow through the shaft to exit at, or adjacent to, the distal end of the shaft. The lumen can be concentric with respect to the shaft. The medical instrument can comprise a deflection structure extending inwardly to direct fluid flow from the lumen across the distal end of shaft. The deflection structure can be ring shaped. The lumen can be offset from an axis of the shaft. The deflection structure can extend inwardly from a rim of the distal end of the shaft.

A surface of the shaft can have one or more protrusions extending radially outwards. The one or more protrusions may be configured, in use, to contact an inner wall of a cannula to allow defining a fluid flow path between the shaft and the cannula. The one or more protrusions can be ribs extending at least partially along the shaft. The ribs can extend substantially along an entire length of the shaft. The one or more protrusions can be arranged substantially uniformly around a circumference of the shaft. The one or more protrusions can be arranged non-uniformly around a circumference of shaft. The one or more protrusions can be different sizes and around a circumference of shaft, wherein the different sizes of the one or more protrusions are configured to create varied gas path sizes. The one or more protrusions can comprise a spiral fin. The shaft can have a cross-sectional shape different from a cross-sectional shape of the cannula. The shaft of the medical instrument can comprise a heating device.

According to another disclosed aspect, there is provided a medical instrument accessory for localizing insufflation or venting of fluid from a distal end of a medical instrument having a shaft. The medical instrument accessory includes a body configured to be mounted to the medical instrument. The body defines an inner lumen dimensioned to receive at least a portion of the shaft, a proximal end, a distal end, and a longitudinal axis between the ends. The distal end defines an opening and is configured to be arranged, in use, at or adjacent the distal end of the medical instrument. The inner lumen is shaped to define a fluid flow path, such that, in use, fluid flows in and/or out of the fluid flow path through the opening.

The medical instrument accessory described in any of the preceding paragraphs may further comprise one or more of the following features. The accessory may include at least one deflection structure arranged to receive fluid flowing through the inner lumen and direct the received fluid to flow transversely to the longitudinal axis. The at least one deflection structure may be arranged to direct fluid to flow out of the opening. The at least one deflection structure may be configured such that, in use, the, or each, deflection structure directs the received fluid to flow substantially across the distal end of the medical instrument. The at least one deflection structure may be configured to, in use, direct the received fluid to flow substantially parallel to the distal end of the medical instrument. The at least deflection structure may be arranged to direct the received fluid to flow substantially perpendicularly to the longitudinal axis. The at least one deflection structure may be arranged to direct the received fluid in a plurality of separate streams. The at least one deflection structure may be arranged such that at least two of the streams are directed to intersect with each other. The at least one deflection structure may be arranged such that each of the at least two of the streams are directed radially towards the longitudinal axis. The at least one deflection structure may be arranged such that at least two of the streams are directed to be parallel to each other. The at least one deflection structure may be arranged such that at least two of the streams are directed to diverge away from each other. The at least one deflection structure may be arranged such that the plurality of streams are directed across two or more planes spaced axially apart from each other. The, or each, deflection structure may be arranged to extend from one half of the inner lumen. The, or each, deflection structure may be arranged to cover equal to, or less than, half of the opening defined by the distal end.

The inner lumen may define two portions, being a first portion defining a first diameter, and a second portion defining a second diameter which is greater than the first diameter. The, or each, deflection structure may extend from the second portion partially across the opening. The first diameter may be dimensioned to be substantially equivalent to an external diameter of the medical instrument such that, in use, the first portion forms a close fit with the medical instrument. The body may comprise a shaft and an end cap releasably securable to the shaft, and wherein the at least one deflection structure is defined by the end cap.

The medical instrument accessory described in any of the preceding paragraphs may include an alignment feature defining a recess shaped to at least partially receive a portion of the medical instrument, and the recess be arranged to inhibit relative rotation of the medical instrument and the accessory. The alignment feature may be arranged at, or adjacent to, the proximal end of the body. The recess may be configured to be an open-ended slot configured to extend along the longitudinal axis. The recess may be defined by a pair of spaced elongate members. The recess may be defined by a shroud shaped to be complementary to, and at least partially surround, the portion of the medical instrument. The body may comprise a shaft and an end cap releasably securable to the shaft, and the alignment feature may extend from the end cap. The alignment feature may be releasably securable to the end cap.

The medical instrument accessory described in any of the preceding paragraphs may include a locking mechanism operable to retain the medical instrument in the accessory. The locking mechanism may include a cam rotatable about an axis between an open position and a locked position, such that, in the locked position during use, the cam is arranged to interfere with the medical instrument. The locking mechanism may be arranged at, or adjacent to, the proximal end of the body. The body may define a first slot and a second slot extending perpendicularly to the first slot and intersecting with the first slot, and the cam may include shaft, and each slot can be dimensioned to receive the shaft. The cam may include a protrusion at each end of the shaft, and the first slot can be dimensioned to receive the shaft and the protrusions, and the second slot can be dimensioned to receive only the shaft such that the protrusions are arranged outside of the second slot to engage the cam with the body.

It will be appreciated that reference to “proximal” and “distal” in this specification are in accordance with the conventional meanings in the art for these words, where the terms are relative to an operator or user of a device. For example, a distal end of a medical instrument is typically the end arranged, in use, to be away from the operator, typically within or against the patient.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the scope of the disclosure. In some cases, a “slice” has been shown for clarity purposes for some sectional and cross-sectional views of a three-dimensional cannula, sheath, or accessory. A person skilled in the art would be able to appreciate that these figures illustrate a slice of a three-dimensional cannula, sheath, or accessory. In some cases, projecting surfaces have not been shown for clarity. For example, projecting hole surfaces are hidden in some views.

FIG. 1 illustrates schematically an example medical gases delivery apparatus in use in surgery.

FIG. 2 illustrates schematically an example medical gases delivery apparatus.

FIGS. 3A-3D illustrate schematic views of an embodiment of a cannula configured to direct the flow of gases within a surgical cavity.

FIGS. 4-5B illustrate the impact on the field of vision within the surgical cavity with the use of localized insufflation and/or localized venting.

FIGS. 6A-6F illustrate an extendable element on a cannula that can be secured to a medical instrument and release or vent gas at the distal end of the medical instrument.

FIGS. 7A-7B illustrate an extension for a cannula to release or vent gas at the distal end of the medical instrument.

FIGS. 8A-8B illustrate a medical instrument accessory that can be attached to a medical instrument.

FIGS. 9A-9D illustrate a medical instrument accessory that can be positioned within a cannula and positioned around a medical instrument.

FIGS. 10A-10B illustrate a medical instrument accessory that can be positioned within a cannula and positioned around a medical instrument.

FIG. 11 illustrates the directed gas flow around the medical instrument within the medical instrument accessory.

FIGS. 12A-12B illustrate ribs located on an inner surface of a medical instrument accessory.

FIGS. 13A-13B illustrate ribs that can be positioned at a distal end of the medical instrument accessory, defining gas flow paths between the ribs.

FIGS. 14A-14B illustrates protrusions that can be positioned at a distal end of the medical instrument accessory.

FIGS. 15A-15C illustrates ribs that can be positioned at a first location at a distal end of the medical instrument accessory and a second spaced-apart location at the proximal end of the medical instrument accessory.

FIG. 16 illustrates a spiral structure that can include spiral fins positioned along the inner wall of the medical instrument accessory.

FIG. 17 illustrates a flexible flaring tip that can be positioned at the distal end of the medical instrument accessory.

FIGS. 18A-18B illustrate a flexible section that can be positioned at the distal end of the medical instrument accessory.

FIGS. 19A-19B illustrate uneven spacing of ribs that can be positioned at the distal end of the medical instrument accessory.

FIGS. 20A-20B illustrate ribs of different widths that can be spaced around the distal end of the medical instrument accessory.

FIGS. 21A-21B illustrate notches that can be positioned along the body of the accessory.

FIGS. 22A-22B illustrate a medical instrument accessory that can have a shape that is non-circular thereby allowing gas flow around the medical instrument.

FIG. 23 illustrates a directed gas flow across the medical instrument within the cannula.

FIGS. 24A-37E include a medical instrument accessory that can have a body that can be positioned over at least a portion of a shaft of the medical instrument and a stopping portion at or adjacent the distal open end and the stopping portion can position an end of the medical instrument shaft adjacent to the open distal end of the medical instrument accessory.

FIGS. 38-40C illustrate a heating device that can be used with any of the directed gases flow cannulas or medical instrument accessories described herein.

FIGS. 41A-41F illustrate various heating methods to integrate into a medical instrument accessory described herein. FIGS. 42A-42F illustrate various power options for providing power to the heating devices.

FIGS. 43A-43C illustrate variations of how heat is transferred to the gas flow that passes by or through the medical instrument accessory.

FIGS. 44A-47 illustrate gas supply options that can be used for any of the cannulas, medical instrument accessories, or medical instruments described herein.

FIGS. 48-52D illustrate attachment options for securement of the device that incorporates a medical instrument accessory and that can be used for any cannulas, medical instrument accessories, or medical instruments.

FIGS. 52E-52F illustrates a locking mechanism at a proximal end of a medical instrument accessory shaft.

FIGS. 53A-53B illustrate a cannula with a gas flow path between the cannula lumen and the medical instrument.

FIGS. 54A-54B illustrate a medical instrument with a medical instrument accessory attachment.

FIGS. 55A-55B illustrate a medical instrument with more than one medical instrument accessory attached to the medical instrument.

FIG. 56 illustrates a medical instrument with a medical instrument accessory attached to the medical instrument.

FIGS. 57A-57B illustrate a medical instrument with a medical instrument accessory.

FIGS. 58A-58B illustrate a medical instrument with a medical instrument accessory.

FIGS. 59A-59B illustrate a medical instrument with a medical instrument accessory.

FIGS. 60A-60B illustrate a cannula with a gas flow path between the cannula lumen and the medical instrument.

FIGS. 61A-61C illustrate an embodiment of a medical instrument with a lumen to direct gas flow through the medical instrument.

FIGS. 62A-62C illustrate an embodiment of a medical instrument with a lumen to direct gas flow through the medical instrument.

FIGS. 63A-63B illustrate protrusions arranged uniformly around the circumference of the shaft of the medical instrument to direct flow concentrically around the medical instrument.

FIGS. 64A-64B illustrate two sets of protrusions arranged uniformly around the circumference of the shaft of the medical instrument to direct flow concentrically around the medical instrument.

FIG. 65 illustrates protrusions in a spiral arrangement around the circumference of the shaft of the medical instrument to direct flow concentrically around the medical instrument.

FIGS. 66A-66B illustrate protrusions arranged non-uniformly around the circumference of the shaft of the medical instrument to direct flow concentrically around the medical instrument.

FIGS. 67A-67B illustrate protrusions with uneven widths around the shaft of the medical instrument to direct flow concentrically around the medical instrument.

FIGS. 68A-68B illustrate a medical instrument with a shaft with a non-circular cross-section.

FIGS. 69A-69C illustrate a medical instrument with a ledge.

FIGS. 70A-70C illustrate a medical instrument with a ledge on one side of the medical instrument to direct gas across the lens.

FIGS. 71A-71C illustrate a medical instrument with a circular ledge around an angled medical instrument to direct gas across the lens in all directions of the ledge plane.

FIGS. 72A-72C illustrate an angled medical instrument with a ledge on one side of the angled medical instrument to direct gas across the lens.

FIGS. 73A-73E illustrate embodiments of power options for a heating device for a medical instrument.

FIGS. 74A-74C illustrate an embodiment of a medical instrument accessory including a deflection structure configured to direct fluid flow relative to the distal end of the accessory.

FIGS. 74D-74I illustrate alternative embodiments of the medical instrument accessory shown in FIGS. 74A-74C. FIG. 75 illustrates a medical instrument accessory with a seal element at the proximal end of the shaft of the accessory to prevent gas from exiting or leaking at the proximal end.

FIGS. 76A-76C illustrate embodiments of an alignment feature configured to inhibit relative rotation of a medical instrument and an accessory for the medical instrument.

FIGS. 77A-76F illustrate an embodiment of a locking mechanism operable to secure an accessory for the medical instrument to the medical instrument.

### DETAILED DESCRIPTION

Although certain embodiments and examples are described below, it will be appreciated that the disclosure extends beyond the disclosed embodiments and/or uses, and includes obvious modifications and equivalents thereof. It is intended that the scope of the disclosure should not be limited by any particular embodiments described below. It will be appreciated that while some features may be disclosed in relation to one or more embodiments, and other features be disclosed in relation to one or more other embodiments, combining these features together in one or more further embodiments is within the scope of the disclosure. It would consequently be understood that any combinations of any disclosed features in an embodiment of a medical instrument accessory, or the instrument itself, is within the scope of the disclosure.

### Example Medical Gases Delivery Systems

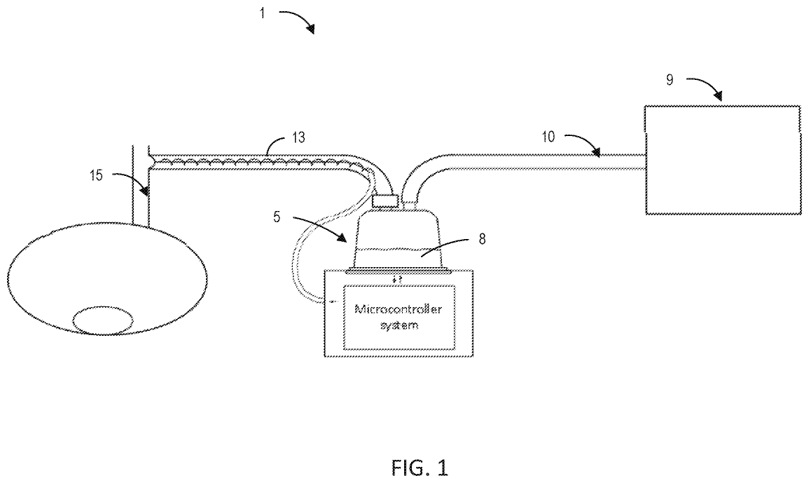

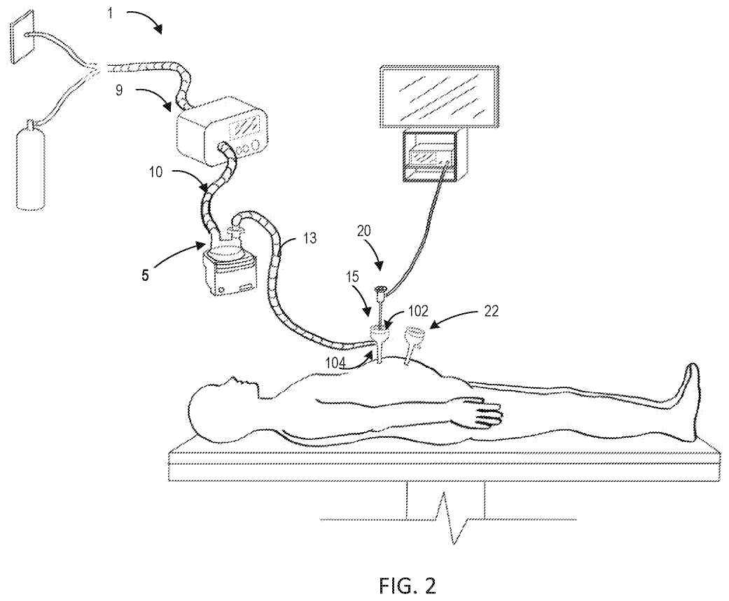

Example surgical systems are shown in FIGS. 1 and 2, which illustrate an insufflation system 1 during a medical procedure. In some embodiments, the system can comprise a fluid source, such as a gas source 9, and a medical instrument configured to be inserted into a surgical cavity within a patient 2, via a cannula 15. The gas source 9 may include any appropriate supply of gases, such as a canister, wall source, and generator, such as a blower. It will be appreciated that a fluid as referred to herein may refer to any gas or liquid. A humidifier, for example a passover humidifier with a humidifier chamber 5 holding a volume of humidification fluid 8, may be located between the gases source and the surgical cavity. The system may have functionality for suction and/or venting of surgical smoke, debris, or the like. This functionality may include one or more venting cannulas 22. The system 1 can include monitoring equipment that is used together with the system. For example, a surgical scope (hereinafter referred to as a “scope”), such as a laparoscope, including a camera, may be used with, or be part of, the medical instrument to allow displaying images recorded by the scope on an external monitor. Optionally, the gases may be delivered via delivery tubes 10, 13 which can heat or cool the gases as they travel between the gases source and the surgical cavity.

The cannula 15 can be used to deliver gases into the surgical cavity. The cannula 15 can include one or more passages to introduce gases and/or one or more medical instruments 20 into the surgical cavity. The medical instrument may be any appropriate instrument for use within the surgical cavity, such as a scope, an electrocautery tool, an electro-surgery tool, an energy, laser cutting and/or cauterizing tool, or the like.

As described herein, a proximal direction with respect to a cannula, medical instrument, or medical instrument accessory, generally refers to an operatively top end of the cannula, instrument, or accessory, while a distal direction with respect to a cannula, instrument, or accessory generally refers to an operatively bottom end of the cannula, instrument, or accessory. The operatively bottom end is generally configured to be the first end inserted into the surgical cavity. More detailed examples of the directed gases flow cannulas and medical instrument accessories are described below. Reference numerals of the same or substantially the same features may share the same last two digits.

### Examples of Medical Instrument Accessory

Condensation occurs when the temperature of a gas falls below the dew point temperature for the level of humidity the gas is carrying. This may be caused by the gas contacting a surface which is at a temperature below the dew-point temperature. Medical instruments intended for insertion into the surgical cavity via the cannula 15, such as cameras and/or surgical scopes, are typically at a temperature lower than the human body. The humidified gases can thus condense on the instrument to form droplets on a lens, and/or elsewhere on the instrument which can drip onto the lens. Similarly, the lens of the instrument may collect other debris and/or fluids, such as bodily fluid and/or tissue, or be clouded by smoke or other debris. Should fluid and/or debris collect on, or in the vicinity of, the lens, this can impede vision of an operator of the instrument, such as a surgeon or other medical personnel participating in the surgery. This may require removing the instrument from the surgical cavity to clean the lens, which can extend duration of the surgical procedure. Furthermore, when the instrument is removed from the cavity, this can cause the temperature of the instrument to decrease, which can result in further fogging and/or condensation when reintroduced to the cavity.

The present disclosure provides examples of a medical instrument accessory which can be used with a cannula 15 of a medical gases delivery or laparoscopic system. The medical instrument accessory is configurable to convey gases relative to a medical instrument, e.g. a scope. This may allow reducing or preventing fluid droplets or debris collecting on the instrument, and/or removing fluid or debris from the instrument.

The example medical instrument accessories disclosed herein can be retro-fitted to existing surgical systems, for example, insufflation systems, without requiring bespoke customization. The example medical instrument accessories disclosed herein can therefore enhance optical clarity of a scope lens and/or maintain a clear field of vision during use. This may aid in minimizing operation duration and post-operation complications (for example, pain, adhesions, and/or others), and/or can make it easier for the medical personnel, such as the surgeon, in navigating the cannula during the medical procedure.

Delivery of gas flow close to or across the distal end of the medical instrument, e.g. scope, may heat the end of the instrument and/or exert force relative to the end. This may inhibit condensation forming by affecting the environment immediately around the scope lens. Additionally or alternatively, this may propel fluid and/or debris away from the end. This may be achieved by manipulating fluid flows, temperatures, and/or humidity in the environment. This can advantageously maintain the temperature of scope lens (or other instrument component, such as a sensor) above the dew point of the gas in the zone adjacent to the scope lens. The medical instrument accessory can be single use (disposable) or reusable. Alternatively, parts of the medical instrument accessory can be single use (disposable) or reusable. The medical instrument accessory may be made of materials that are biocompatible and/or sterilizable.

It will be appreciated that disclosed embodiments of medical instrument accessories may comprise features and/or integers disclosed herein or indicated in the specification individually or collectively, and that any and all combinations of two or more disclosed features is within the scope of the disclosure.

The example directed gases flow cannulas can have any of the features of the cannula 15. For example, the directed gases flow cannula can have a cannula body 102 connectable to an elongate shaft 104. The elongate shaft 104 can optionally have a pointed end for easier insertion of the cannula 100 into the surgical cavity. In some cases, the elongate shaft 104 of the cannula can be utilized in combination with an obturator to function as a trocar. A trocar can include a cannula and an obturator. The cannula body 102 can have a guiding feature to aid insertion of the medical instruments into the cannula. As used herein, a guiding element, guiding feature, guide element, and/or guide feature can be used interchangeably herein to refer to a feature used to aid insertion, or provide support for or position a medical instrument within a cannula.

A surgical, for example, insufflation system for supplying insufflation gases to a surgical cavity, such as any surgical, for example, insufflation systems disclosed herein, can incorporate any of the example medical instrument accessories disclosed herein. As described above, the system can include a gas source configured to provide the insufflation gases, a humidifier in fluid communication with the gas source and configured to humidify insufflation gases received from the gas source., A gases delivery tube may extend between, and be in fluid communication with, the humidifier and the cannula.

During laparoscopic surgery, there will generally be some form of electrosurgery/electrocautery/ultrasonic or laser device surgery to cause cutting or coagulation within the insufflated surgical cavity. This can produce surgical smoke which can concentrate within the cavity, especially when there are no significant gas leaks or suction/irrigation. A high concentration of smoke in the insufflated cavity, or a smoke plume moving towards a lens of a scope in the cavity, can significantly impede optical clarity and field of vision for an operator of the scope, such as a surgeon or other member of a surgical team. In the absence of venting or suction, surgeons typically release all, or a portion of, the gas from inside the cavity, then re-insufflate.

Directing gas flow relative to a lens of a scope can advantageously mitigate the effect of concentrated smoke in the insufflated cavity by affecting the environment immediately adjacent to the lens. For example, this may propel the smoke away from the scope to clear the line of sight and enhance the surgeon's field of vision. This may also prevent a smoke plume from contacting the medical instrument.

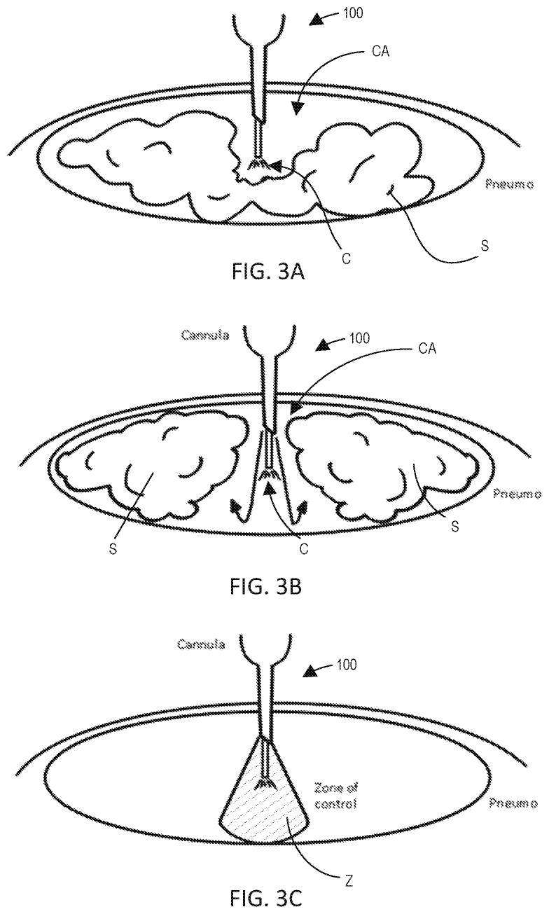

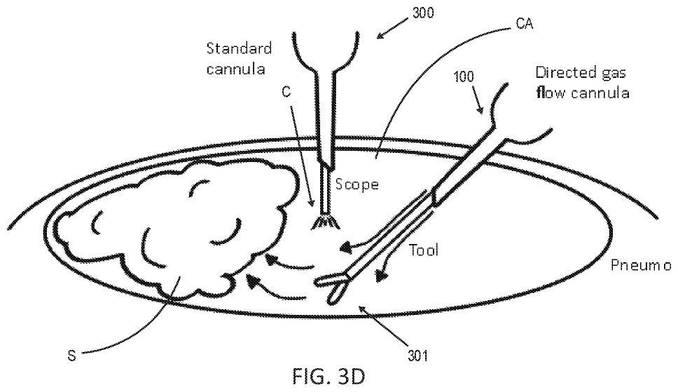

FIG. 3A schematically illustrates a surgical cavity, showing a cannula 100 extending inside an insufflated cavity CA, such as within the pneumoperitoneum, and a scope lens C at a distal end of a scope inserted through the cannula 100. Surgical smoke S is shown surrounding the scope lens C. FIG. 3B schematically illustrates the surgical cavity scenario of FIG. 3A with directed gas flow, illustrated by arrows, moving the smoke S away from the scope lens C. As illustrated, the scope can be held concentric with the cannula 100 and the gases be directed substantially concentric and coaxial with the instrument. The gases are directed around and past the scope as they travel through the cannula 100. FIG. 3C illustrates schematically that the cannula can include one or more features as described in greater detail below to create a zone of control Z in which smoke, fluid, or other unwanted media is propelled away from the medical instrument. In other words, in some cases a gas barrier or envelope, also referred to herein as a gases shroud, gases sheath, protection zone, or region of controlled temperature and humidity, can be formed by directing gas flow through the cannula 100, such that gases flow from an opening, through a lumen of the cannula 100, and exit from one or more outlets. FIG. 3D schematically illustrates the scenario of FIGS. 3A-3C showing the directed gases flow cannula 100 used in combination with a second cannula 300. In some cases, a first medical instrument can include a scope lens C and can be inserted through the second cannula 300 and the directed gases flow can be introduced through the directed gases flow cannula 100. As illustrated in FIG. 3D, the directed gases flow cannula 100 can provide directed gas flow, indicated by arrows, within the insufflated cavity CA to cause the smoke S to move away from the scope lens C inserted through the second cannula 300. In some cases, the directed gases flow cannula 100 can also support a second medical instrument 301 that can be inserted through the directed gases flow cannula 100 as illustrated in FIG. 3D.

The directed gases flow cannula 100 can be configured to create a gases envelope extending distally beyond the end of an associated medical instrument, and/or onto or past a portion of the medical instrument, such as an endoscope lens, sensor, or other element for example. The gases envelope formed could have any number of potential advantages, including one or more of, but not limited to: maintaining the temperature of the instrument above a dew point; preventing or reducing fogging and/or condensation forming on the instrument; reducing or preventing smoke, debris or other unwanted media from contacting or collecting on the instrument; directing the smoke, debris or other unwanted media away from the instrument and/or an outlet of a lumen such that a gas envelop disperses the smoke plume; substantially surrounding a portion of the instrument (or substantially the entire instrument portion positioned within the surgical cavity and/or the cannula shaft); concentrically surrounding the instrument inside the shaft and/or distally beyond the outlet of the shaft; extending a predetermined or calculated distance in a desired direction beyond the outlet; and maintaining a temperature, humidity, and/or pressure controlled environment about the shaft (e.g., distal end of the shaft) and outlet of the elongate shaft, such as maintaining the temperature in the envelope above a dew point.

The gases flow separates and diverges from the scope surface at a distance from the outlet of the cannula 100. The distance and jet divergence angles do not necessarily depend on scope insertion depth. The distance at which the flow separates and diverges from the scope may decrease proportionally to increasing flow rate. In some embodiments, the extension of the gases envelope can be controlled to extend to about, no more than about, or at least about 10 mm, 25 mm, 50 mm, 75 mm, or 100 mm, or more or less past the distal end of a medical instrument and/or luminal outlet, or ranges including any two of the foregoing values. In some configurations, the gases envelope can extend beyond the distal end and/or luminal outlet at any distance between about 10 mm and about 100 mm. In some configurations, the gases envelope can extend no more than about 100 mm past the distal end of a medical instrument and/or luminal outlet. The distance the envelope extends can be based on the flow rate of gases delivered.

The surgical system, for example, insufflation system, can be configured to deliver intermittent (e.g., cyclic) and/or constant flow of gases. In some embodiments, a constant flow provides a more stable envelope. In other embodiments, an intermittent or cyclic flow allows for an envelope being formed to cause droplets on the scope to evaporate. The flow rate of the gases delivered can be sufficient to maintain a pressurized surgical cavity. The flow rate can be, for example, at least about 2 liters per minute (lpm). In one example the flow rate provided is at least about 6 lpm. In one example the flow rate provided is at least about 7 lpm. In another example the flow rate is at least about 10 lpm or between about 10 lpm and about 12 lpm, or about, at least about, or no more than about 2, 4, 6, 8, 10, 12, 14, 16, 20, 30, 40, 50, 60, or more or less lpm, or ranges incorporating any two of the foregoing values. The flow rates can be any suitable flow rate. In one example the flow rate can be as high as between about 40 L/min to about 50 L/min, or more. Further the flow limit is based on the pressure in the surgical cavity. The pressure in the surgical cavity can be defined in regulatory standards, e.g. established clinical practice, and for example can be up to about 50 mmHg in some cases. The example flow rates listed above can be continuous flow rates. If an intermittent flow rate is delivered, the flow rate can vary between an upper and a lower value, including values listed for continuous flow rates. Where the instrument is arranged concentrically with the cannula 100, this can enhance the insufflation gases being distributed around, and/or against, the instrument, e.g. a lens of a scope. This also allows for defogging of the instrument. In general, increasing the flow rate of the insufflation gases can reduce the required defogging time. Cold, dry gas provided to the cannula 100, while the instrument is held concentric, can also help to defog the lens C. The defogging can be improved with warming of gases. This can be achieved using a humidifier such as the SH870 humidifier from Fisher & Paykel Healthcare (Auckland, NZ) which can further humidify the gases. Humidifying the gases has advantages of reducing cell/tissue damage. A larger flow rate provides an increased distance such that the envelope covers the scope as the scope is inserted beyond the cannula. The distance between the end of the shaft and the distal end of the scope can be referred to as the insertion depth. The insertion depth can be, for example, between about 20 mm and about 100 mm. The insertion depth can be, for example, up to about 80 mm. The defogging time may increase as the insertion depth extends beyond a threshold distance, such as for example 100 mm in some cases. The flow rate from the gas source 9, e.g., insufflator, can be controlled to vary the length of the envelope. The flow may be controlled at the insufflator or there may be a flow control device positioned in the gases path or the humidifier may include a device or structures to control the flow rate delivered to the cannula.

### Examples of Localized Flow for Insufflation or Venting

In some cases, a localized flow of gases for insufflation or venting near the field of vision of a scope lens can be used to clear stagnation zones and debris, including but not limited to, smoke. For example, during laparoscopic surgery, there will generally be some form of electrosurgery or electrocautery within the surgical cavity. This can produce, for example, surgical smoke which can concentrate within the cavity, especially when there are no significant gas leaks or suction. A high concentration of smoke in the surgical cavity, or a smoke plume moving towards a lens of a scope in the cavity, can severely impede optical clarity and field of vision for an operator of the scope. In some cases, surgeons may vent or use suction to extract the smoky gas and/or reduce the concentration of smoke through insufflating the cavity with clean gas.

In some cases, a localized insufflation flow adjacent the lens of the scope can reduce or eliminate stagnation zones of gas flow around the scope. This can help move the gas within the field of vision and/or dilute the smoke with clean insufflation gas, both of which can improve the optical clarity. This may cause clean insufflation gas to be pushed into the field of vision.

In some cases, a localized venting adjacent the lens of the scope can effectively remove smoky gas by venting gas from close to the smoke source. This can allow removing gas from, or close to, the field of vision.

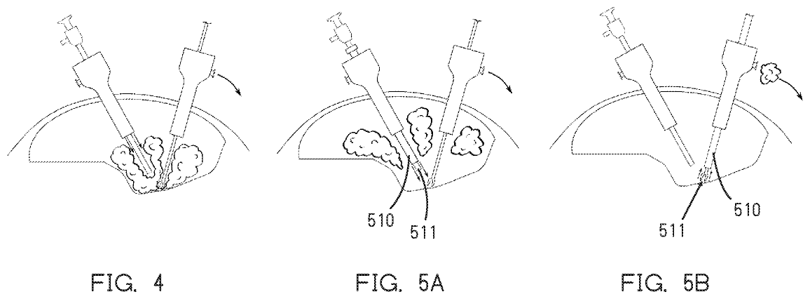

FIGS. 4-5B illustrate localizing conveying gases to, and venting gases from, adjacent the distal end of the medical instrument, e.g. a scope. This can advantageously allow affecting the environment immediately surrounding the lens, regardless of depth of insertion of the medical instrument into the cavity and beyond the distal end of the cannula. In some embodiments, systems can be configured to arrange the medical instrument to be concentric relative to the accessory. Furthermore, some embodiments are configured to direct gases relative to the lens, such as across or at the lens. Other embodiments are configured to direct gases in front of the lens and/or away from the lens.

FIG. 4 illustrates a surgical cavity with no intervention. FIG. 5A illustrates the use of a first medical instrument accessory 510 to provide localized insufflation relative to the distal end of a medical instrument 511 which includes a scope or camera. FIG. 5B illustrates the use of the first medical instrument accessory 510 to provide localized venting relative to the distal end of the medical instrument 511.

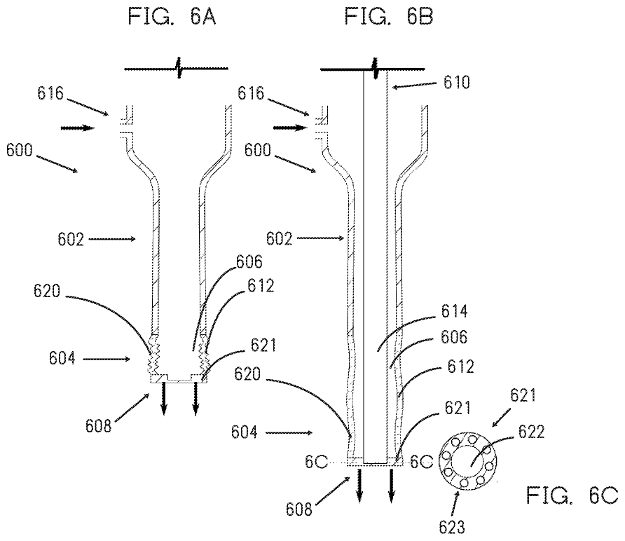

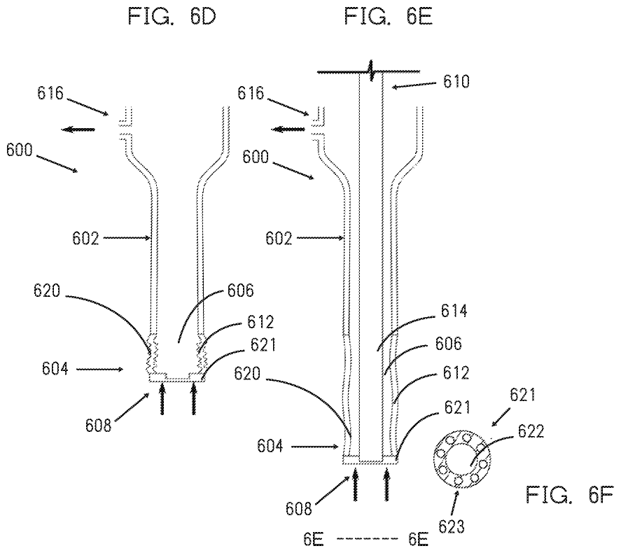

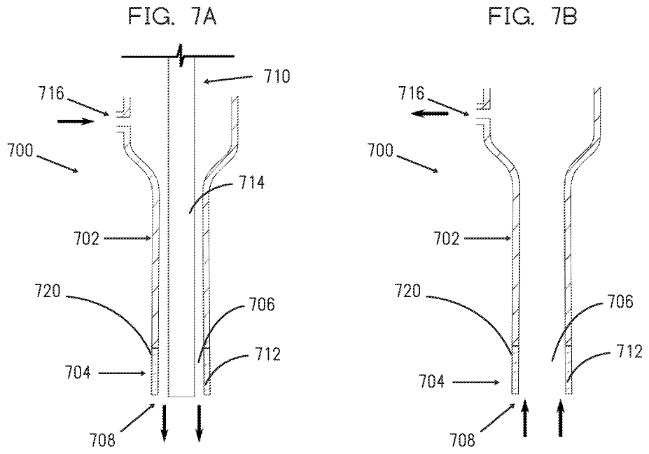

FIGS. 6A-6F illustrate a medical instrument accessory including an extendable element 620 removably attached to a cannula 600 configured to convey gas relative to the distal end of a medical instrument 610. FIGS. 6A and 6B illustrate the cannula 600 and extendible element 620 conveying gas past the instrument 610 to be expelled from a distal end 608 adjacent a distal end of the instrument 610, the gas indicated by single-ended arrows. FIGS. 6D and 6E illustrate the cannula 600 and extendible element 620 receiving gas into the distal end 608 and conveying the gas past the instrument 610, the gas indicated by single-ended arrows.

FIG. 6A illustrates a body 604 of the extendable element 620 in a compressed configuration to be withdrawn towards an elongate shaft 602 of the cannula 600. The body 604 is dimensioned to at least partially receive a shaft of the medical instrument 610. The body 604 may define one or more lumens. In the illustrated embodiment, the body 604 defines inner lumen 606. The one or more lumens can extend from and be in fluid communication with an opening or outlet defined by a distal end 608 of the body 604. The lumen 606 is defined by an inner sidewall 612 of the body 604. FIG. 6B illustrates the extendible element 620 and cannula 600 receiving the medical instrument 610, such as a scope or camera. The instrument 610 has an elongate shaft 614 arranged within the lumen 606 of the body 604. In the illustrated embodiment, the distal end 608 is perpendicular to an axis of the body 604 and shaft 602. In other embodiments, the distal end 608 is arranged at an angle to the axis of the body 604, for example, to receive an angled scope. The cannula 600 can include a gas port 616 configurable to allow gas to enter or exit the cannula 600. The gases inlet port 616 can be connected to a gases delivery tube of a gas source, for example, an insufflation system (such as any of the systems disclosed herein, for example). In some embodiments, the shaft 602 of the cannula 600 is elongate, and may be cylindrical, generally cylindrical, or otherwise tubular. In some embodiments, a gas flow path can be at least partly defined by an inner wall of the shaft 602 and the outer wall of a shaft of the medical instrument 610. The extendible element 620 may be removably attached to the distal end of the cannula 600, as shown in FIGS. 6A-6F. In other embodiments, the extendible element 620 and cannula 600 are integral. The extendible element 620 is deformable, being at least partially flexible, and can be deformed between a retracted position, shown in FIG. 6A, and an extended position, shown in FIG. 6B. In some embodiments, the retracted position is the default or resting position of the extendable element 620.

In some cases, the medical instrument 610 can be a laparoscope, and fluid is conveyed by the cannula 600 and/or extendible element 620 to exit and/or enter the fluid flow path adjacent a lens of the laparoscope. In other cases, the medical instrument can be an electrocautery tool.

The extendable element 620 can define an abutment structure, such as a ring 621, at the distal end to allow abutting against the distal end of the instrument 610. It will be appreciated that shaping the abutment structure as the ring 621 is exemplary and that the structure can be alternatively configured, such as comprising an alternative shelf, shoulder or other surfaces. In the illustrated embodiment, the ring 621 is dimensioned to partially receive the end of the instrument 610, and can include a catch, or include a mechanism to allow being releasably securable to the medical instrument 610. Securing the extendable element 620 to the instrument 610 allows the element 620 to be extended as the medical instrument 610 is passed through the lumen 606. This allows the extendable element 620 to extend proportionally to the insertion depth of the instrument 610.

FIG. 6C illustrates a cross-section through line 6C-6C of FIG. 6B, showing the distal end of the extendable element 620 through the ring 621. The ring 621 defines an opening 622 to allow light to access the medical instrument 610. The ring 621 is dimensioned to prevent the medical instrument 610 from passing through the opening 622. The ring 621 can define additional apertures 623. In the illustrated embodiment, these apertures 623 are arranged in an annular array spaced circumferentially about the opening 622. In other embodiments, the ring 621 defines more or less of the apertures 623. In use, the apertures 623 can allow gas to pass through the ring 621 to exit from the body 604 and into the surgical cavity, or be vented from the surgical cavity and into the body 604.

FIGS. 6D-6E illustrate the cannula 600 and extendible element 620 being used to vent gas from the surgical cavity, through the distal end 608, and out of the gas port 616.

FIGS. 7A and 7B illustrate a medical instrument accessory comprising an extension 720 securable to a cannula 700 to allow expelling or venting gas adjacent to a distal end of a medical instrument 710 inserted through the cannula 700. FIG. 7A illustrates a body 704 of the extension 720 attached to a distal end of the cannula 700. The cannula 700 can include an elongate shaft 702. The body 704 can be mountable to, or positioned over, at least a portion of the shaft 702. The body 704 may define one or more lumens, such as inner lumen 706 illustrated in FIGS. 7A and 7B. The one or more lumens can extend from and be in fluid communication with an opening defined in a distal end 708 of the body 704. The lumen 706 is defined by a sidewall 712 of the body 704. The lumen 706 is configured to receive at least a portion of an elongate shaft 714 of the medical instrument 710. The body 704 defines a free end arranged perpendicular to an axis of the body 704. In other embodiments, the free end is arranged at an angle to the axis. The cannula 700 can include a gas port 716 arranged to convey gas into, or out of, the cannula 700. The port 716 is connectable to a gases delivery tube of a gas source, for example, an insufflation system (such as any of the systems disclosed herein, for example). FIG. 7A illustrates the cannula 700 and extension 720 arranged to allow gas to be expelled adjacent to the distal end of the medical instrument 710, as shown by the arrows extending from the distal end 708 showing the gases flow path.

The extension 720 can be attached or secured to the end of the cannula 700. The extension 720 can allow a focused gas flow to be delivered to an area of interest, such as within the field of vision of the medical instrument 710.

FIG. 7B illustrates the cannula 700 and extension 720 being used to vent gas from the surgical cavity, as shown by the arrows pointing inward at the distal end 708.

The distal end 708 of the extension 720 can be arranged at or adjacent the distal end of the medical instrument 710. In some embodiments, the extension 720 is cylindrical, or otherwise shaped to fit to the cannula 700 and/or shaped to be complementary to the instrument 710, such as the inner wall 712 being configured to be offset a defined distance from the external surface(s) of the instrument 710. In some cases, the gas flow path can be at least partly defined by the inner wall of the extension 720 and the outer wall of the shaft of the medical instrument 710. The extension 720 is configured to attach to the distal end of the cannula 700 as shown in FIGS. 7A-7B.

### Examples of Medical Instrument Accessory for Directed Gas Flow

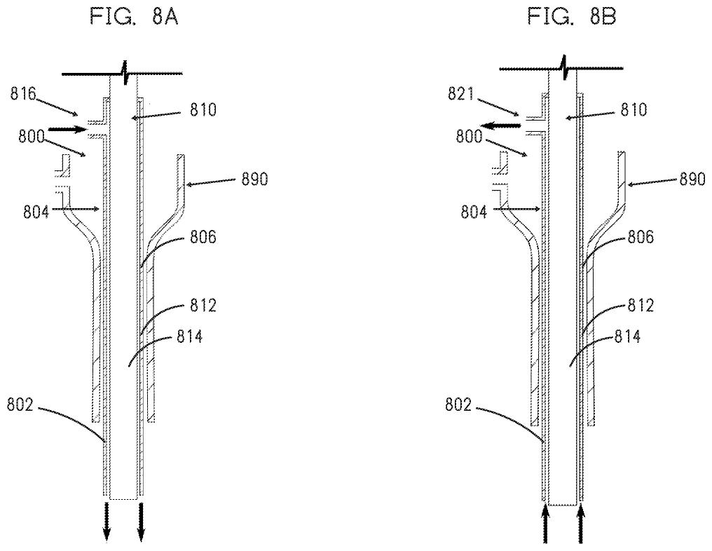

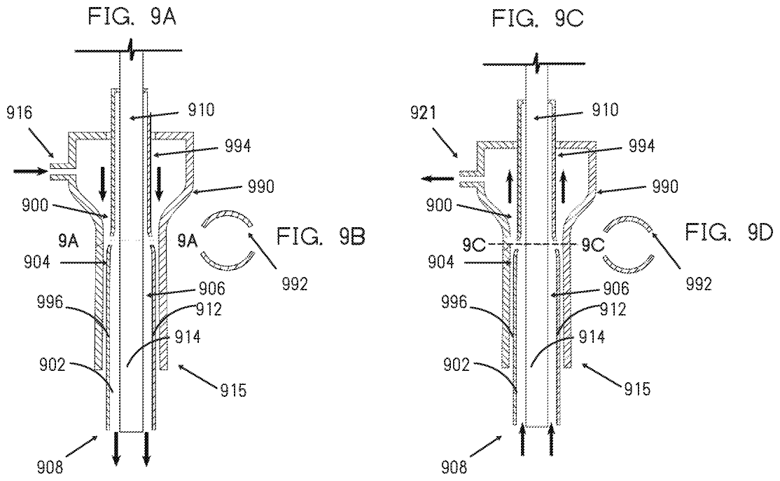

FIGS. 8A and 8B illustrate a medical instrument accessory 800 that can be positioned around a medical instrument 810. The medical instrument accessory 800 and the medical instrument 810 can be positioned within a cannula 890. The medical instrument accessory 800 is securable to the medical instrument 810, allowing connecting the accessory 800 and instrument 810 prior to insertion into the cannula 890, and providing direct insufflation to release gas adjacent to the tip of the medical instrument 810 regardless of insertion depth relative to the cannula 890.

The medical instrument accessory 800 includes a body 804 dimensioned to be positioned within a lumen of the cannula 890 when mounted on the medical instrument 810, such as scope. The body 804 can include an elongate shaft 802 defining one or more lumens. In the illustrated embodiment, the shaft 802 defines an inner lumen 806 dimensioned to at least partially receive a shaft 814 of the medical instrument 810. The one or more lumens can extend from, and be in fluid communication with, an opening or outlet defined in a distal end of the body 804. The lumen 806 is at least partially defined by an inner sidewall 812 of the body 804. The body 804 can include a gas port 816 configured to allow gas to enter and/or exit from the body 804. The port 816 can be connected to a gases delivery tube of a gas source, for example, insufflation system (such as any of the systems disclosed herein, for example). FIG. 8A illustrates the medical instrument accessory 800 attached to the medical instrument 810 to allow gas to be directed parallel to the shaft 814 and/or away from the distal end of the shaft 814, creating a gas flow path as shown by the arrows. In the illustrated embodiment, gas is delivered into the accessory 800 through a port, configured in this illustrated embodiment as a gas inlet port 816. In other embodiments, the proximal end of the body 804 can be in fluid communication with the gas source. The accessory 800 can be attached to the medical instrument 810 to inhibit gas from leaking between the accessory 800 and the instrument 810. In some cases, the accessory 800 includes a sealable attachment mechanism to sealably secure the accessory 800 to the instrument 810. The accessory 800 can be dimensioned to extend to, or beyond, the distal end of the medical instrument 810 and can be secured to release gas at the end of the medical instrument 810 regardless of the depth of the medical instrument 810. The accessory 800 can allow a focused gas flow to be delivered closer to an area of interest. As shown in FIG. 8A, the accessory 800 can release the gas closer to the field of vision of the medical instrument 810. In some cases, the body 804 can have a length that extends to the distal end or beyond the distal end of the medical instrument 814 such that distal end of the body 804 is adjacent to or extends beyond an distal end of the medical instrument as shown in FIGS. 8A-8B.

FIG. 8B illustrates the medical instrument accessory 800 configured to vent gas at any depth of insertion of the medical instrument 810 near the field of vision as noted with the arrows pointing inward at the distal end of the accessory 800. FIG. 8B illustrates the port configured as a gas venting port 821 that allows the gas to vent out of the assembly.

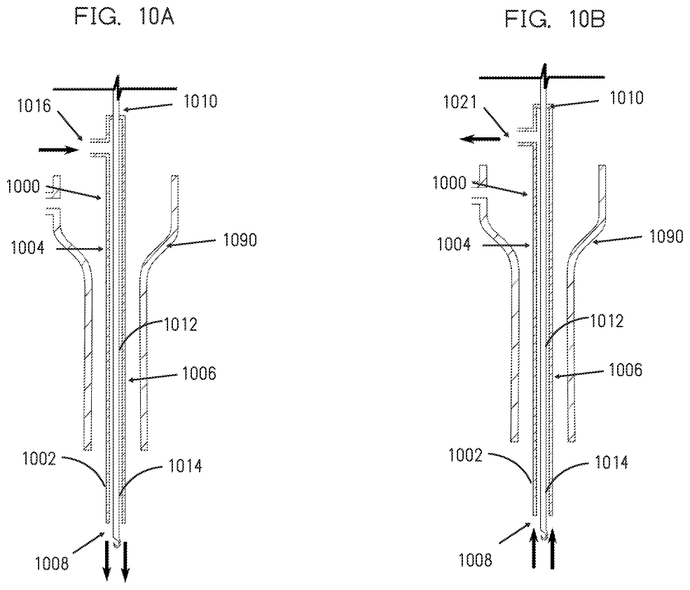

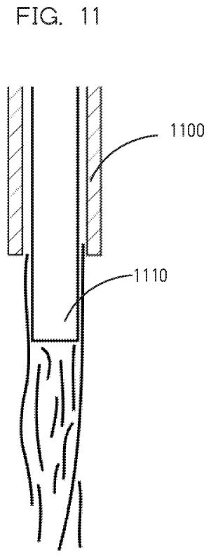

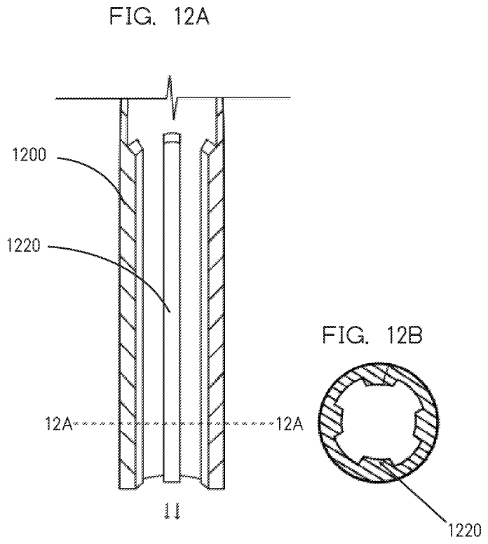

FIGS. 9A-9D illustrate a medical instrument accessory 900 that can be positioned around a medical instrument 910. The medical instrument accessory 900 and the medical instrument 910 can be positioned within a cannula 990. The medical instrument accessory 900 can be attached to the medical instrument 910 prior to insertion into the cannula 990. The medical instrument accessory 900 can allow channeling insufflation to release gas at and/or beyond the distal end of the medical instrument. The medical instrument accessory 900 can extend to the distal end of the medical instrument as shown in FIGS. 9A-9B.

FIG. 9A illustrates a medical instrument accessory 900 which can include a body 904. The medical instrument accessory 900 can be positioned within a lumen of a cannula 990 as illustrated in FIGS. 9A-9B. The body 904 can include an elongate shaft 902. One, two, or more lumens can be present in the body 904, e.g., inner lumen 906. The one, two, or more lumens can extend from and be in fluid communication with an opening or outlet at or near distal end 908 of the body 904. A lumen 906 can be defined by a sidewall, such as inner sidewall 912 of the body 904. An elongate shaft 914 of the medical instrument 910 can be located within the lumen 906 of the body 904. The cannula 990 can include a gases inlet port 916, which can be connected to a gases delivery tube of a surgical, for example, insufflation system (such as any of the systems disclosed herein, for example).

FIG. 9A illustrates the medical instrument accessory 900 with the medical instrument 910 located therein, allowing a release of gas and gas flow at the distal end of the medical instrument shaft 914 as shown by the arrows. The accessory 900 can secure or attach to a proximal end of the medical instrument 910 to prevent gas from leaking out the proximal end and to release gas at the distal end of the accessory 900. In some cases, the attachment can be sealing. In some cases, the attachment can prevent fluid loss at the attachment. The gas can enter through the gases inlet port 916 into the cannula 990.

As shown in FIG. 9B, the accessory 900 can include one or more apertures 992 in the body that allow gas to pass from the cannula 990 into the lumen 906 of the accessory 900. The gas can be channeled through the accessory apertures 992. The gap between the interior wall of the cannula 990 and the exterior wall of the accessory 900 can be small enough so that the gas can be channeled through the accessory 900 as shown in FIG. 9A. Gas can be released at the distal end of the accessory 900 at or adjacent to the field of vision of the medical instrument 910. The accessory 900 can be inserted into the cannula lumen and the at least one aperture 992 preferably does not extend past the distal end of the cannula 915 so as to allow a desired or suitable flow of gases through lumen 906. In some cases, the position of apertures 992 defines the length of functional range of the shaft. The accessory 900 can have a longitudinal length such that the open distal end of the accessory 900 can be located at or adjacent the distal end of the medical instrument 910 and can release gas at the end of the medical instrument 910 regardless of the depth within the surgical cavity.

As shown in FIG. 9A and 9C, the body can have a first portion 994 at the proximal end of the accessory 900 having a lumen with a first diameter comparable with the medical instrument outer wall diameter. The body can have a second portion 996 at a distal end of the accessory 900 having a lumen with a second diameter greater than the medical instrument outer wall diameter. The body can have at least one aperture 992 in fluid communication with gas flow path. FIG. 9B illustrates a cross-section through line 9A-9A of FIG. 9A at the location of the apertures 992. FIG. 9B illustrates the apertures in the medical instrument accessory 900 that allow gas to enter the accessory thereby channeling the gas into the lumen of the medical instrument accessory closer to the medical instrument. As shown in FIG. 9A and 9C, the diameter of the lumen can transition from the first diameter to the second diameter. The diameter can transition at or adjacent the position of the aperture on the body. In some cases, locating the apertures 992 at the transition can bias the gas flow into the aperture 992 and through the gas flow path between the body and the medical instrument 910.

FIG. 9C illustrates a medical instrument accessory 900 to vent gas near the field of vision as noted by the arrows indicating flow direction. The accessory 900 shown in FIG. 9C is similar to the medical instrument accessory 900 shown in FIG. 9A except the medical instrument accessory 900 vents gas near the field of vision of the medical instrument 910 and includes a venting port 921. The medical instrument accessory 900 can be placed on any medical instrument to vent gas near the field of vision. For example, electrocautery cutting can produces smoke which can cause vision problems. Therefore, if the medical instrument accessory is placed on the cutting tool, any smoke generated will be blown away or vented before causing problems, such as vision impairment. The gas can be vented close to the field of vision and pass through the lumen of the accessory 900. As illustrated by the arrows in FIG. 9C, the gas can pass through the apertures 992 in the accessory 900 and into the interior of the cannula and out of the venting port 921. FIG. 9D illustrates a cross-section through line 9C-9C of FIG. 9C of the medical instrument accessory 900 at the location of the apertures 992. FIG. 9D illustrates the apertures in the medical instrument accessory 900 that allow gas to pass from the accessory 900 into the cannula 990.

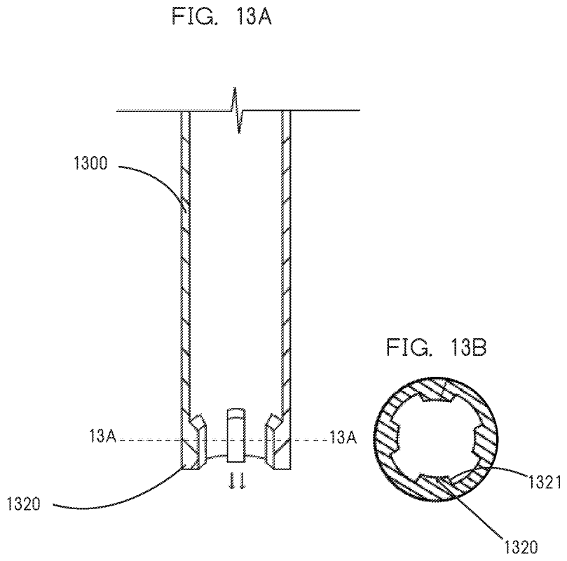

FIGS. 10A-10B illustrate a medical instrument accessory 1000 that can be positioned around a medical instrument 1010. The medical instrument accessory 1000 and the medical instrument 1010 can be positioned within a cannula 1090. The medical instrument accessory 1000 can be attached to the medical instrument 1010 prior to insertion into the cannula 1090. The medical instrument accessory 1000 can be placed on the medical instrument 1010 to channel gas through the medical instrument accessory 1000. The distal end of the medical instrument accessory 1000 can be positioned in use at or adjacent the distal end of the medical instrument 1010 as shown in FIGS. 10A-10B. In some cases, a portion of the medical instrument 1010 can be located outside the open distal end of the accessory. In some cases, the medical instrument 1010 can be placed into the accessory prior to both being introduced into the cannula 1090.

FIG. 10A illustrates a medical instrument accessory 1000 which can include a body 1004. The medical instrument accessory 1000 can interact with or be secured to the medical instrument 1010. The medical instrument accessory 1000 and medical instrument 1010 assembly can be positioned within a cannula 1090. The accessory 1000 can be positioned within a lumen of a cannula 1090 as illustrated in FIGS. 10A-10B. One, two, or more lumens can be present in the body 1004, e.g., inner lumen 1006. The one, two, or more lumens can be in fluid communication with an opening or outlet at or near distal end 1008 of the body 1004. A lumen 1006 can be defined by a sidewall, such as inner sidewall 1012 of the body 1004. The medical instrument 1010, such as an electrocautery device, or an endoscope or camera or other scope device in other embodiments, for example, including an elongate shaft 1014 of the medical instrument 1010 can be received within the lumen 1006 of the body 1004.

The accessory 1000 can include a gases inlet port 1016. The gases inlet port 1016 can be connected to a gases delivery tube of a surgical, for example, insufflation system (such as any of the systems disclosed herein, for example). FIG. 10A illustrates a medical instrument accessory 1000 with the medical instrument 1010 inserted within the accessory 1000 allowing a release of gas at the distal end of the medical instrument shaft 1014 as shown by the gas flow path arrows extending from the distal end of the accessory 1000. The medical instrument accessory 1000 can be positioned within the cannula 1090. The accessory 1000 can be secured to a proximal end of the medical instrument 1010. A sealing means or seal element can be provided to prevent gas from leaking out the proximal end and to release gas at the distal end of the accessory 1000. The gas can enter through the gases inlet port 1016 into the body 1004 of the accessory 1000. The accessory 1000 can allow gas flow to be delivered closer to the area of interest. The accessory 1000 can release gas near the distal end or area of interest of the medical instrument 1010. The accessory 1000 can be placed on any medical instrument 1010. For example, electrocautery cutting can produce smoke which can cause vision problems. Therefore, if the accessory 1000 is engaged with the cutting tool, generated smoke can be vented away before causing any problems.

FIG. 10B illustrates a medical instrument accessory 1000 that allows gas to enter near the distal end or area of interest of a medical instrument as indicated by the arrows pointing towards the distal end of the accessory 1000. FIG. 10B illustrates a medical instrument accessory 1000 similar to the medical instrument accessory 1000 shown in FIG. 10A except the medical instrument accessory 1000 allows gas to enter near the distal end or area of interest of the medical instrument 1010 and includes a venting port 1021.

The accessory 1000 can vent gas near the field of vision. The accessory 1000 can be placed on any tool. For example, electrocautery cutting can produce smoke and vision problems. Therefore, if the accessory 1000 is placed on the cutting tool, any smoke generated can be vented before causing any problems. The gas can be vented close to the field of vision and pass through the lumen of the accessory 1000. As illustrated by the arrows in FIG. 10B, the gas can pass through the lumen of the accessory 1000 and exit out of the venting port 1021.

### Examples of Directed Gas Flow around a Medical Instrument with a Medical Instrument Accessory

It can be desirable to create a controlled micro-environment around the lens or working end of the device to overcome some of the condensation, fogging, or other issues that can cause reduced visibility. A directed gas flow around the medical instrument can allow for the creation of the micro-environment to be controlled around the lens or working end of the device. This environment can isolate the lens from the warm and humid environment of the pneumo-peritoneum. The medical instrument with the lens can be either held concentrically or off-axis surrounded by a gas pathway. This may cause the insufflation gas to conform to, and may substantially enclose, the medical instrument. The gas may then cover the lens of the medical instrument and, to a certain extent, form a barrier between the lens and the surrounding environment. If the delivered gas conditions are controlled, this can allow affecting the environment around the medical instrument.

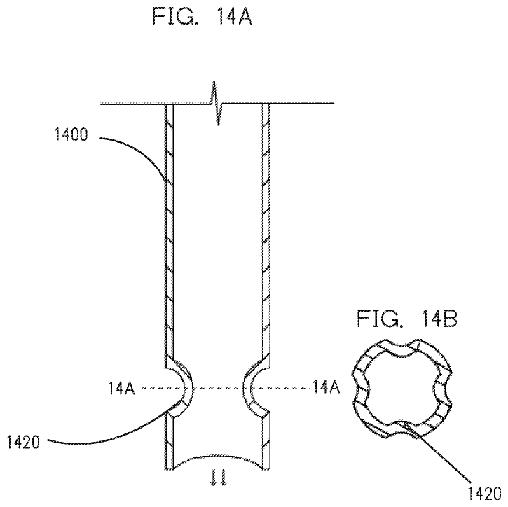

FIG. 11 illustrates the directed gas flow around the medical instrument 1110 within the medical instrument accessory 1100.

In some cases, a medical instrument accessory can include at least one structure or guide element to position the medical instrument relative to the lumen, such as concentric to a longitudinal axis defined by the lumen, or offset from the axis, such that a gas flow path is defined between the lumen inner wall and the medical instrument shaft. More detailed examples of the guide elements or structures that may also be used in combination or in addition to those described herein are described in International Application No. PCT/NZ2019/050100, titled “DIRECTED GAS FLOW SURGICAL CANNULA FOR PROVIDING GASES TO A PATIENT,” filed on Aug. 16, 2019, the disclosures of which are hereby incorporated by reference in their entirety.

In some cases, the structure or guide elements can be arranged at an inner wall of the accessory, such as being defined by the inner wall, or being mountable to or adjacent the inner wall. For example, the structure or guide elements may be in the form of one or more members which are separate from, and securable to, a shaft of the accessory to be arranged at the inner wall. The structure or guide elements can extend inwardly relative to the inner wall such that, in use, the structure or guide elements are positioned between the inner wall and the medical instrument. In some cases, the medical instrument accessory can have a plurality of the structures. The proximal end of the body of the medical instrument accessory can be in fluid communication with a gas source or vent. Gas can be directed out from the open distal end of the accessory and around an end of the medical instrument. In some embodiments, the at least one structure or guide element is formed by one or more ribs. FIG. 12A illustrates longitudinal (axially) extending ribs 1220 defined by an inner wall of a medical instrument accessory 1200. The ribs 1220 are arranged to concentrically position the accessory 1200 around a medical instrument (not shown). FIG. 12B illustrates a cross-section through line 12A-12A of FIG. 12A of the medical instrument accessory 1200. As shown in FIG. 12B, the ribs 1220 can form an annular array spaced circumferentially around the inner wall to hold a medical instrument substantially concentrically in the medical instrument accessory 1200. FIGS. 12A and 12B illustrate the ribs 1220 extending partway along the longitudinal length of the inner wall of the accessory 1200. It will be appreciated that the ribs 1220 may be arranged only adjacent one, or both, of the proximal and distal ends of the accessory 1200, continuously between the ends, or discontinuously at spaced intervals between the ends. The ribs 1220 are arranged such that gases can flow through the accessory 1200, between the ribs 1220, and around the medical instrument. In some cases, the ribs 1220 can extend inwardly from an inner wall of the medical instrument accessory 1200 as shown in FIG. 12B. It will further be appreciated that configuring the instrument supporting structures or guide elements as ribs 1220 is exemplary and these structures may be configured in other forms, such as dimples, fins, splines, grooves, or channels.

As illustrated in FIG. 13A, ribs 1320 can be positioned at, or adjacent, an open distal end of a medical instrument accessory 1300. The ribs 1320 can define a gas pathway for gas to travel around the periphery or circumference of the medical instrument. The ribs 1320 can hold the medical instrument substantially concentrically within the lumen of the medical instrument accessory. In some cases, the ribs may not contact the medical instrument while in use, but can act as limits or stops, to prevent the medical instrument from contacting the interior sidewall of the medical instrument accessory shaft. FIG. 13B illustrates a cross-section through line 13A-13A of FIG. 13A of the medical instrument accessory 1300. As shown in FIG. 13B, the ribs can be spread radially to promote centering of the medical instrument in the medical instrument accessory 1300 and the gas flow can pass between the sidewall 1321 of the ribs 1320 around the medical instrument and the outer wall of the medical instrument (not shown). In some cases, the ribs 1320 can be spaced axially and/or longitudinally apart regularly or irregularly along the inner circumference of the medical instrument accessory 1200.

In some cases, the at least one structure or guide element can include protrusions such as bumps or indents in the medical instrument accessory to direct gas flow concentrically around the medical instrument. The protrusions can extend inwardly from the inner wall of the lumen of the medical instrument. The protrusions can be located at any position along the body of the medical instrument accessory to direct the flow concentrically around the medical instrument. The protrusions can be located at a proximal end, distal end, or intermediate portion along the length of the lumen of the accessory. As illustrated in FIG. 14A, the protrusions 1420 can be positioned at an open distal end of the medical instrument accessory 1400 and gas can pass between the protrusions 1420 concentrically around the medical instrument (not shown). FIG. 14B illustrates a cross-section through line 14A-14A of FIG. 14A of the medical instrument accessory 1400. As shown in FIG. 14B, the protrusions 1420 can be spread radially to hold a medical instrument concentrically in the medical instrument accessory 1400 and the gas flow can pass between the protrusions 1420 around the medical instrument. In some cases, the protrusions can be spaced uniformly (or substantially uniformly) around the diameter of the accessory lumen. In other embodiments, the protrusions can be spaced non-uniformly around the diameter of the accessory lumen creating gas flow paths of different sizes.

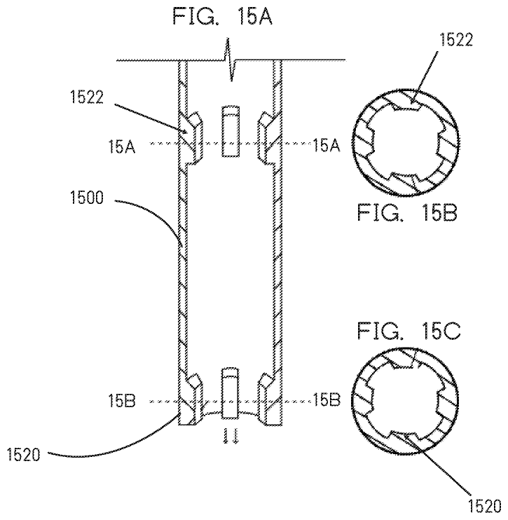

In some cases, the at least one structure can include more than one set of ribs on the medical instrument accessory to direct gas flow concentrically around the medical instrument. The more than one set of ribs can be located at any position along the body of the medical instrument accessory to direct the flow concentrically around the medical instrument. As illustrated in FIG. 15A, the ribs 1520 can be positioned at a first location at a distal end of the medical instrument accessory 1500. Ribs 1522 can be positioned in a second location proximal to the first location on the medical instrument accessory 1500. The gas can pass between the ribs 1520 and ribs 1522 concentrically around the medical instrument (not shown). FIG. 15B illustrates a cross-section through line 15A-15A of FIG. 15A of the medical instrument accessory 1500 through ribs 1522. FIG. 15C illustrates a cross-section through line 15B-15B of FIG. 15A of the medical instrument accessory 1500 through ribs 1520. As shown in FIGS. 15B and 15C, the ribs 1520 and ribs 1522 can be spread radially at the two locations to hold the medical instrument concentrically in the medical instrument accessory 1500 and the gas flow can pass between the ribs 1520 and ribs 1522 around the medical instrument. In some cases, the ribs 1520 and ribs 1522 can be positioned along the accessory at any number of locations any number of times. In some cases, the ribs can be located adjacent the proximal end of the medical instrument accessory.

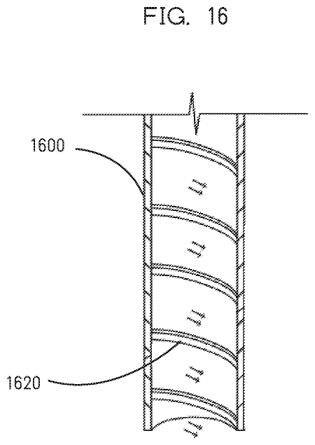

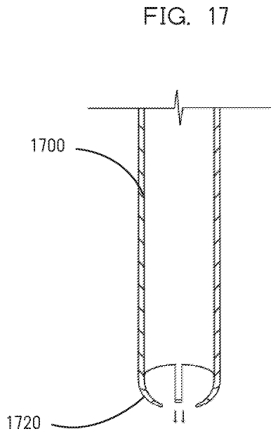

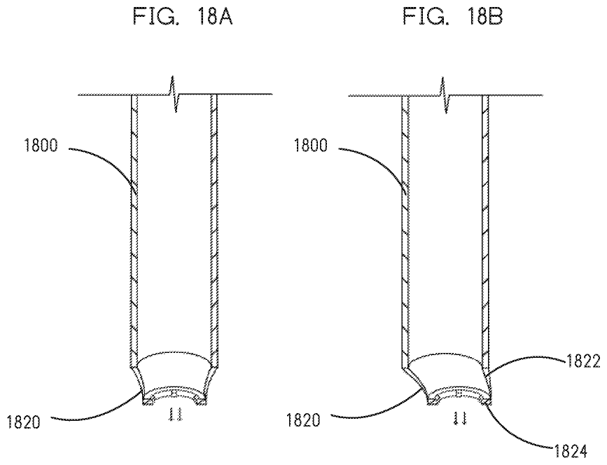

The at least one structure can include one or more fins extending inwardly from the inner wall of the accessory. The one or more fins can be in a spiral structure on the medical instrument accessory to direct gas flow in a vortex pattern around the medical instrument. The spiral structure can be located at any position along the body of the medical instrument accessory to direct the flow in a vortex pattern around the medical instrument. As illustrated in FIG. 16, the spiral structure can include spiral fins 1620 positioned along the inner wall of the medical instrument accessory 1600. The spiral fins 1620 can direct flow as well as holding the medical instrument concentrically within the medical instrument accessory 1600.

The at least one structure or guide element can include a flexible or semi-flexible flaring tip at the distal end of the medical instrument accessory to direct gas flow concentrically around the medical instrument and position the medical instrument concentrically within the accessory. The flexible flaring tip can be located at a distal end of the body of the medical instrument accessory to direct the flow concentrically around the medical instrument. As illustrated in FIG. 17, the flexible flaring tip 1720 can be positioned at the distal end of the medical instrument accessory 1700. The flaring tip 1720 can be made of thin plastic or another semi-flexible material at the distal end of the accessory 1700. The flexible flaring tip 1720 can flare out when the medical instrument (not shown) is pushed past the flaring tip 1720. The flexible flaring tip can push back on the medical instrument holding the medical instrument concentrically within the medical instrument accessory 1700.

The at least one structure or guide elements can include a flexible section at the distal end of the medical instrument accessory to direct gas flow concentrically around the distal end of the medical instrument. The flexible section can be located at a distal end of the body of the medical instrument accessory to direct the flow concentrically around the medical instrument and hold the medical instrument in the medical instrument accessory. As illustrated in FIGS. 18A-18B, the flexible section 1820 can be positioned at the distal end of the medical instrument accessory 1800. In some cases, the flexible section 1820 can include a flexible bellows. The flexible section can be made of a flexible material 1822 attached to the body of the medical instrument accessory 1800 and can have a non-flexible tip or edge 1824 that attaches to the medical instrument. The flexible section 1820 can have a movable tip that can move with the medical instrument and maintain the flow around the lens of the medical instrument as shown in FIG. 18B. As illustrated in FIG. 18A-18B, the movable tip can have a flexible material 1822 and a solid tip or edge 1824 with one or more protrusions extending radially inwardly. The solid tip or edge 1824 can be laterally movable and can be substantially parallel with the open distal end of the accessory. The solid tip or edge 1824 can engage with an end of the medical instrument.

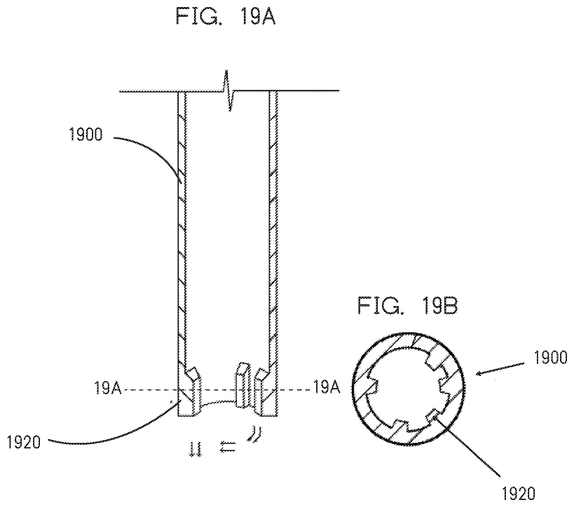

The at least one structure can include ribs or channels at the distal end of the medical instrument accessory to direct gas flow concentrically around the medical instrument. The ribs or channels can be similar to the ribs or channels described with reference to FIGS. 12A-16. The ribs or channels can be located at a distal end of the body of the medical instrument accessory to direct the flow concentrically around the medical instrument and hold the medical instrument in the medical instrument accessory. In some cases, the ribs and channels can be even sized ribs spaced unevenly around the accessory. The one or more ribs can create a channel of gas flow between adjacent ribs. As illustrated in FIGS. 19A-19B, the ribs 1920 can be positioned at the distal end of the medical instrument accessory 1900. The ribs 1920 can be unevenly spaced around the accessory 1900 to deflect contamination or water droplets away from the lens or encourage evaporation of condensation/fogging that has formed on the lens as illustrated by the arrow shown at the distal end of the accessory 1900 in FIG. 19A. The ribs 1920 can be located at any point along the accessory 1900 or the full length of the accessory 1900. The ribs 1920 can be spaced unevenly around the accessory 1900 to direct an entrainment of gas over the medical instrument. Uneven spacing of the ribs 1920 can create different size channels around the inner wall of the accessory 1900. The different sized channels can result in different gas speeds and may create pressure differentials at the distal end of the accessory 1900. FIG. 19B illustrates a cross-section through line 19A-19A of FIG. 19A, illustrating the uneven spacing of ribs 1920 around the inner wall of the accessory 1900. The ribs 1920 can hold the scope substantially concentrically within the accessory 1900.

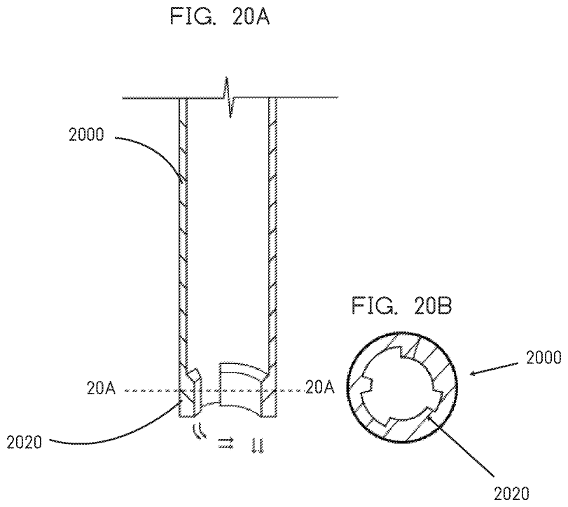

In some cases, the ribs and channels can be ribs of different sizes spaced around the accessory. The one or more ribs can define a channel of gas flow between adjacent ribs. As illustrated in FIGS. 20A-20B, the ribs 2020 can be positioned at the distal end of the medical instrument accessory 2000. The ribs 2020 can have different sizes covering different portions around the circumference of the inner sidewall of medical instrument accessory 200. The ribs 2020 can be of different sizes and spaced around the accessory 2000 to entrain gas flow over the medical instrument, shown by the arrows at the distal end of the accessory 2000 in FIG. 20A. Flow across the lens can advantageously deflect contamination or water droplets away from the lens or encourage evaporation of condensation/fogging that has formed on the lens. The ribs 2020 can be located at any point along the accessory or the full length of the accessory. Different size channels can be created by the different width of the ribs around the inner wall of the accessory. The different sized channels can have different gas speeds and create pressure differentials at the distal end of the device. As illustrated in FIG. 20B, which is a cross-section through line 20A-20A of FIG. 20A, the ribs 2020 can be of different widths spaced around the inner wall of the accessory and can hold the scope concentrically within the accessory. Although the ribs are shown at the distal end, the ribs can be of any length.

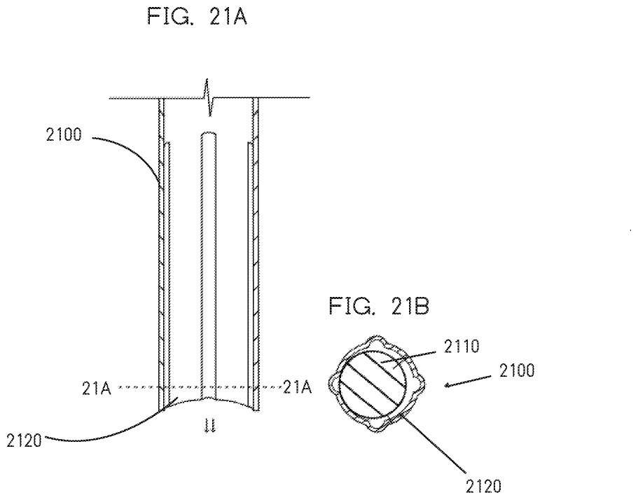

The at least one structure can include notches or channels in the inner wall of the body of the medical instrument accessory to direct gas flow concentrically around the medical instrument. The notches or channels in the body of the medical instrument accessory can define channels for gas flow no matter where the medical instrument is positioned within the accessory. Depending on the axial position of the medical instrument in the accessory, the gas will either flow concentrically around the medical instrument or be entrained over it. As illustrated in FIGS. 21A-21B, the notches or channels 2120 can be positioned substantially along the length of body of the accessory 2100. As illustrated in FIG. 21B, which is a cross-section through line 21A-21A of FIG. 21A, the notches or channels 2120 can allow gas to continue flowing around the medical instrument 2110 even if the medical instrument 2110 is pressed against inside an inside wall of accessory 2100. Although the notches or channels 2120 are shown evenly spaced and extending a large portion of the body of the accessory, the notches or channels can be of any length and can be spaced evenly or unevenly. In some cases, the notches or channels can extend the entire length or substantially the entire length of the lumen of the accessory.

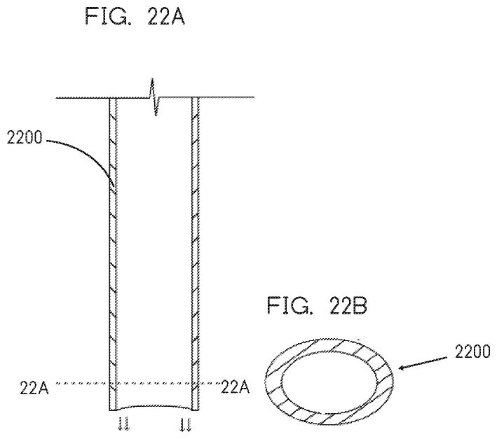

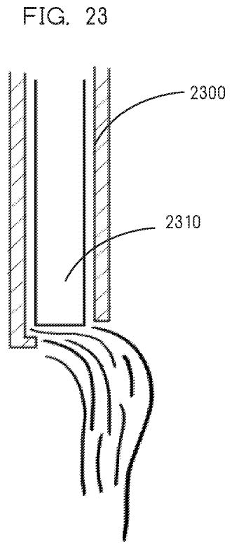

The at least one structure can include a non-circular cross-section in the body of the medical instrument accessory. The medical instrument can have a circular or substantially circular shaft. Therefore, no matter the positioning of the medical instrument within the medical instrument accessory, gas can flow through the accessory. As illustrated in FIGS. 22A-22B, the medical instrument accessory 2200 can have a shape that is non-circular thereby allowing gas flow around the medical instrument. Depending on the axial positioning of the scope, the gas can either flow concentrically around the medical instrument or be entrained over the medical instrument. FIG. 22B illustrates a cross-section through line 22A-22A of FIG. 22A of the medical instrument accessory 2200 the body of the medical instrument. As illustrated in FIG. 22B, the non-circular shape of the accessory 2200 can allow gas to flow around the medical instrument in any position. Although the non-circular cross-section of the body of the accessory is shown as an oval shape, the non-circular cross-section of the body of the accessory can be any non-circular shape.

### Examples of Directed Gas Flow Across a Medical Instrument with a Medical Instrument Accessory

FIG. 23 illustrates a medical instrument accessory 2300 configured to direct fluid flow across a distal end of a medical instrument 2310 arranged within the medical instrument accessory 2300.

In some applications, it can be useful to control a micro-environment surrounding a lens or working end of a medical instrument to mitigate problems created by collection of condensation, smoke or debris near or across the lens/working end. Directed gas flow across the lens or working end of the medical instrument can help to control the micro-environment by affecting the zone around the lens/working end, such as by manipulating fluid flows, temperatures, and/or humidity. For example, directing gas or other fluid around the lens can maintain lens temperature above the dew point of the gas in the local environment to inhibit condensation forming. Also, should moisture or debris collect on the lens, this can be at least partially removed by directing fluid, such as non-saturated gas, across the lens. Some disclosed embodiments, discussed below, are configured to position the medical instrument concentrically, or off axis, and direct gas flow across the lens. The medical instrument can be positioned relative to the distal end of the assemblies described herein to direct flow across the lens at the distal end of the medical instrument shaft.

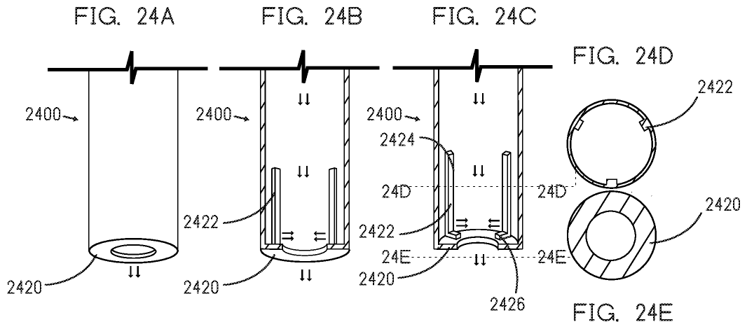

FIGS. 24A-37E illustrate examples of embodiments of a medical instrument accessory having features similar to embodiments shown in FIGS. 6A-22B. These embodiments also include a stopping portion arranged at, or adjacent to, the distal open end. The stopping portion can aid positioning a distal end of a medical instrument, such as a lens of a scope, relative to a distal end of the accessory, for example, to be adjacent, or at a pre-determined distance from, the open distal end of the accessory. In use, the stopping portion can include any features that position the end of the medical instrument shaft a predetermined distance away from the open distal end.

FIGS. 24A-24E illustrate a medical instrument accessory 2400 including a stopping portion 2426 and a deflection structure configured to redirect fluid travelling longitudinally through the accessory 2400 to travel at least partially radially, as illustrated by the single ended arrows. In this embodiment, the deflection structure is configured as an annular ledge 2420 arranged at, or adjacent to, a distal end of the accessory 2400, to define an opening at the distal end. In other embodiments, the deflection structure comprises one or more specifically angled and/or positioned surfaces defined by one or more other structures. The annular ledge 2420 defines a plane and can direct gas across a lens of a medical instrument arranged in the accessory 2400 in all directions of the plane. As shown by the arrows in FIG. 24A-24C, the gas can be directed down the lumen and directed across the lens concurrently with exiting directly into the surgical cavity. In this embodiment, the ledge 2420 extends radially inwards from an edge or rim of the distal end. The ledge 2420 can be substantially perpendicular to the edge of the distal end of the accessory 2400. The accessory 2400 can also include one or more protrusions, in this embodiment in the form of ribs 2422, arranged to position the medical instrument within the accessory 2400. The ribs 2422 may also direct fluid flow through the channel defined between ribs 2422, the medical instrument, the inner wall of the accessory, and the ledge 2420. The ribs 2422 can have a longitudal rib portion 2424 arranged to maintain the position of the medical instrument relative to the longitudinal axis of the accessory 2400 and/or a radial rib portion 2426 (positioned on the ledge 2420) to act as the stopping portion and maintain the position of the medical instrument relative to the distal end of the accessory 2400. The ribs 2422 can be arranged on the inner wall of the lumen of the accessory 2400. FIG. 24D, which is a cross-section through line 24D-24D of FIG. 24C, illustrates the annular arrangement of the ribs 2422 about the inner wall. FIG. 24E is a cross-section through line 24E-24E of FIG. 24C, illustrating the distal end and annular ledge 2420 of the medical instrument accessory 2400.

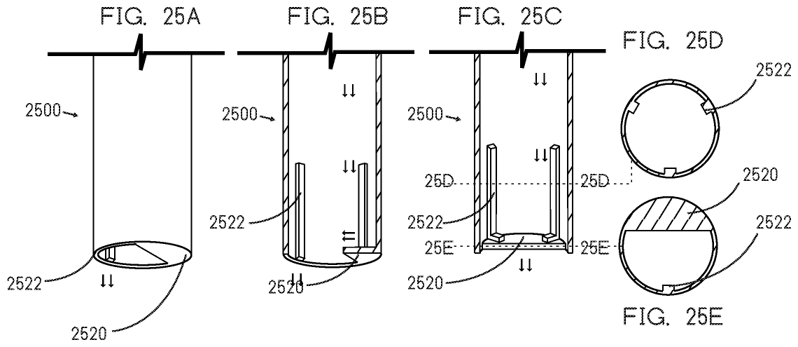

FIGS. 25A-25E illustrate a medical instrument accessory 2500 including a deflection structure arranged to direct fluid flow relative to a distal end of the accessory 2500, the deflection structure in this embodiment in the form of a shelf 2520 extending part way across an opening defined at the distal end of the accessory 2500. In some cases, the medical instrument accessory 2500 can accommodate a medical instrument to be center or offset relative to its longitudinal axis, and has a flat lens or angled lens. The medical instrument accessory 2500 can provide better control of the medical instrument. The shelf 2520 can direct gas across the lens of the medical instrument. As shown by the arrows in FIG. 25A-25C, the gas can be directed down the lumen and be deflected by the shelf 2520 to travel across the lens simultaneously with the gas exiting the distal end and into the surgical cavity. The accessory 2500 can include the shelf 2520 extending part way across the distal opening of the accessory 2500. In some cases, the shelf 2520 can be a segment that extends partially across the distal opening and may define an area of less than half of the opening. For example, the straight edge of the shelf 2520 can be less than the diameter of the open distal end. The shelf 2520 can extend radially inwards from the edge of the open distal end. The accessory 2500 can also include one or more protruding ribs 2522 arranged to be adjacent, or abut, the medical instrument to allow supporting the instrument during use. The ribs can be arranged on the inner wall of the lumen of the accessory. The ribs can be arranged concentrically or substantially concentrically around the inner wall of the lumen of the accessory. The one or more protruding ribs 2522 can direct the flow across the lens through the channel created by the protruding ribs 2522, the medical instrument (not shown), the inner wall of the accessory, and the shelf 2520. The ribs 2522 can be concentric around the scope to let gas flow directly into the body cavity as well as across the lens. The protruding ribs 2522 of the ledge allows the medical instrument to rest on the ribs and the ledge can direct flow to pass across the lens and the distal end of the shaft of the medical instrument. FIG. 25D illustrates a cross-section through line 25D-25D of FIG. 25C that runs through the medical instrument accessory 2500. FIG. 25D illustrates the ribs 2522 in the medical instrument accessory. FIG. 25E illustrates a cross-section through line 25E-25E of FIG. 25C that passes through the distal end of the medical instrument accessory 2500. FIG. 25E shows the shelf 2520 covering one side of the opening of the medical instrument accessory 2500. As shown in FIG. 25E, the shelf 2520 can be a semicircle or have a generally semi-circular shape. In other embodiments, the shelf 2520 defines a crescent shape, or one or more other shapes depending on the requirements of directing fluid flow relative to the distal end of the accessory 2500.

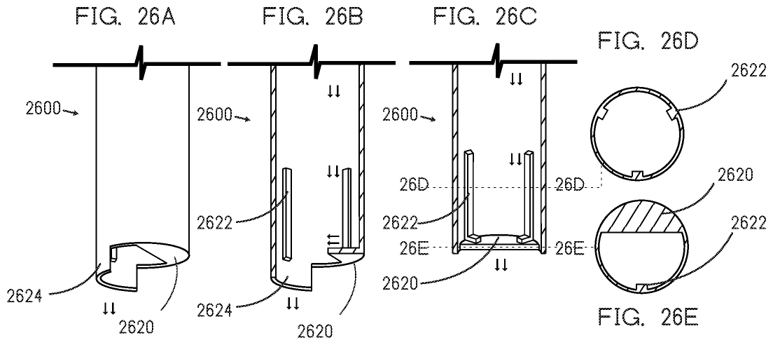

FIGS. 26A-26E illustrate a medical instrument accessory 2600 having a deflection structure configured similarly to the previously described embodiments as a ledge 2620 arranged at one side of the distal end of the accessory 2600. A protuberance extends longitudinally from the distal end to form a wall, in this embodiment being configured as a flange 2624. The ledge 2620 can extend part way across the distal opening of the accessory 2600 to direct gas across the lens of the medical instrument while the gas is exiting the accessory 2600 into the body cavity. The flange 2624 can be located substantially opposite the ledge 2620 as shown in FIGS. 26A-26B. Gas can pass through the lumen, against the ledge 2620 to be directed across the distal end of the medical instrument, and then against the flange 2624 to be directed away from the accessory 2600 and into the surgical cavity, as shown by the arrows in FIGS. 26A-26C. The ledge 2620 can be located on one side of the distal end of the accessory 2600 and the flange 2624 on the opposite side. The ledge 2620 can extend radially inwards from the edge of the open distal end opposite the flange 2624. The ledge 2620 can be on an opposite side of the accessory 2600 than the flange 2624. The flange 2624 can extend the length of a first side of the accessory 2600. A second, opposite side of the accessory 2600 can be shorter than the first side and terminate at the ledge 2624, as shown in FIG. 26B.

The accessory 2600 can also include one or more protruding ribs 2622 arranged to position the medical instrument within the accessory 2600. The ribs can be arranged on the inner wall of the lumen of the accessory. The ribs can be arranged concentrically, or substantially concentrically, around the inner wall of the lumen of the accessory. The one or more protruding ribs 2622 and the ledge 2620 can direct the flow across the lens through a channel created between the ribs 2622, the medical instrument (not shown), the inner wall of the accessory, and the ledge 2620. The ribs 2622 can be concentric around the scope to let gas flow directly into the body cavity as well as across the lens. The protruding ribs 2622 arranged on the ledge 2620 are arranged to support the medical instrument and allow fluid flow past the instrument to be deflected by the ledge and to pass across the distal end of the medical instrument. FIG. 26D, which is a cross-section through line 26D-26D of FIG. 26C, illustrates one possible arrangement of the ribs 2622 in the accessory 2600. FIG. 26E, which is a cross-section through line 26E-26E of FIG. 26C, illustrates the distal end of the medical instrument accessory 2600. FIG. 26E illustrates a cross-section through line 26E-26E of FIG. 26C and shows the ledge 2620 that extends part way across the distal opening of the medical instrument accessory 2600. In some cases, the ledge 2620 can be a segment that extends across the distal opening and is smaller than half of the opening. For example, in this embodiment the ledge 2620 defines a straight edge dimensioned to be less than the diameter of the open distal end. It will be appreciated, that the ledge 2620 may define one or more curved edges across the open distal end, or can be configured as a doubly-curved (three-dimensionally curved) surface arranged partially across the distal end.

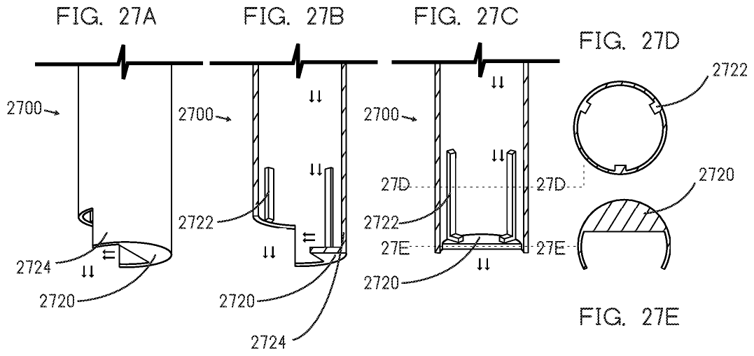

FIGS. 27A-27E illustrate a medical instrument accessory 2700 with a protuberance, configured as a flange 2724, extending longitudinally from a distal end of the accessory 2700 and a deflection structure, configured as a ledge 270, extending from the flange 2724. The ledge 2720 can direct fluid flow relative to the distal end, such as directing gas across a distal end/lens of a medical instrument arranged within the accessory 2700. The ledge 2720 can extend radially inward from the end of the flange 2724 as shown in FIGS. 27A-27B. A cut-out is defined on the opposite side of the distal end from the flange 2724 to allow fluid to exit the accessory 2700. The accessory 2700 can also include one or more protruding ribs 2722 arranged to position the medical instrument. The ribs can be arranged on the inner wall of the lumen of the accessory. The ribs can be arranged concentrically or substantially concentrically around the inner wall of the lumen of the accessory. The one or more protruding ribs 2722 and the ledge 2720 can direct the flow across the lens through the channel defined between the protruding ribs 2722, the medical instrument (not shown), the inner wall of the accessory, and the ledge 2720. The ribs 2722 can be concentric around the scope to let gas flow directly into the body cavity as well as across the lens. The protruding ribs 2722 arranged on the ledge 2720 can support the medical instrument and allow fluid to flow around the instrument to pass across the its distal end. Gases can pass down the lumen and can be directed across the distal end of the accessory 2600, and consequently the medical instrument, by the ledge 2720. Gases can also flow downwards into the surgical cavity after exiting the distal end of the accessory 2700, as shown by the arrows in FIGS. 27A-27C. FIG. 27D, which is a cross-section through line 27D-27D of FIG. 27C, illustrates the arrangement of the ribs 2722 in the accessory 2700. FIG. 27E, which is a cross-section through line 27E-27E of FIG. 27C, shows the ledge 2720 as a segment part of the distal opening of the accessory 2700.

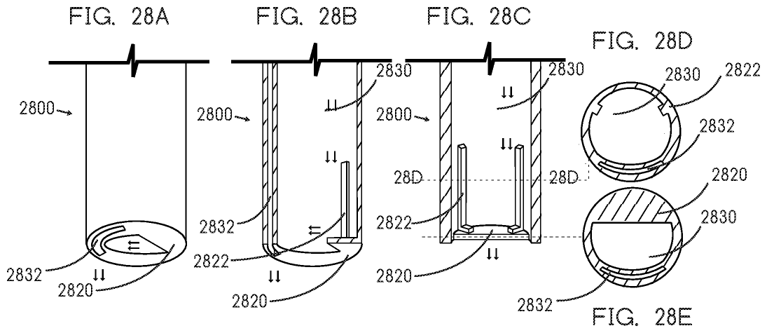

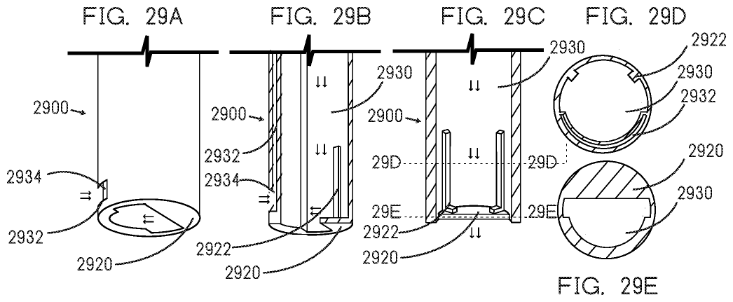

FIGS. 28A-28E illustrate a medical instrument accessory 2800 having an internal sidewall defining a first longitudinal lumen. The accessory 2800 includes a deflection structure, in the form of a ledge 2820, extending part way across a distal end of the accessory 2800. A second longitudinal lumen 2832 is defined at one side of the accessory 2800 and may be in fluid communication with the first lumen. The ledge 2820 can be perpendicular to the inner sidewall. The ledge 2820 is arranged to direct gas flow relative to the distal end, allowing gas flow across a lens of a medical instrument arranged within the first lumen. The ledge 2820 can extend radially inward from the end of the primary or first lumen 2830 as shown in FIGS. 28A-28B. The medical instrument (not shown) can be positioned within the first lumen 2830. As shown by arrows in FIG. 28A-28C, the gas can pass down the lumen and be directed across the distal end of the accessory by the ledge 2820 and will pass over the distal end of the medical instrument as the gas exits into the surgical cavity. The second lumen 2832 can channel insufflation gas to the surgical cavity directly. The accessory 2800 can also include one or more protruding ribs 2822 arranged to position the medical instrument within the first lumen. The ribs can be arranged on the inner wall of the first lumen 2830 of the accessory. The ribs can be arranged concentrically or substantially concentrically around the inner wall of the first lumen of the accessory. The one or more protruding ribs 2822 and the ledge 2820 can direct the flow across the lens through the channel created by the protruding ribs 2822, the medical instrument (not shown), the inner wall of the accessory, and the ledge 2820. The ribs 2822 can be concentric around the scope to let gas flow directly into the body cavity as well as across the lens. The protruding ribs 2822 arranged on the ledge 2820 can support the medical instrument during use and assist directing gas flow to pass longitudinally around the instrument and across the distal end of the instrument. FIG. 28D, which is a cross-section through line 28D-28D of FIG. 28C, illustrates the ribs 2822 in the accessory 2800 and the first lumen 2830 and second lumen 2832. FIG. 28E that passes through the distal end of the medical instrument accessory 2800. FIG. 28E, which is a cross-section through line 28E-28E of FIG. 28C, shows the ledge 2820 covering part of one side of the distal opening of the first lumen 2830 of the accessory 2800, with the second lumen 2832 located opposite to the ledge 2820.

FIGS. 29A-29E illustrate a medical instrument accessory 2900 with a deflection structure, in the form of a ledge 2920, on one side of the distal end of the accessory 2900 and a venting lumen 2932 terminating at a vent port 2934 defined at an opposed side of the accessory 2900. The ledge 2920 can direct gas across the lens of the medical instrument. The ledge 2920 can extend radially inward from the end of the primary or first lumen 2930 as shown in FIGS. 29A-29B. The medical instrument (not shown) can be positioned within the first lumen 2930. As shown in FIG. 29A-29B, the gas can be directed across the distal end of the accessory by the ledge 2920 and will pass over the distal end medical instrument. The venting lumen 2932 can vent gas out of the field of vision to remove any smoke that may be causing optical problems. The accessory 2900 can include a ledge 2920 extending radially inwards from an edge of the distal end of the accessory 2900. The accessory 2900 can also include one or more protruding ribs 2922 arranged to position the medical instrument within the accessory 2900. The ribs can be arranged on the inner wall of the first lumen 2930 of the accessory. The ribs can be arranged concentrically or substantially concentrically around the inner wall of the first lumen of the accessory. The one or more protruding ribs 2922 and the ledge 2920 can direct the flow across the lens through the channel created by the protruding ribs 2922, the medical instrument (not shown), the inner wall of the accessory, and the ledge 2920. The ribs 2922 can be concentric around the scope to let gas flow directly into the body cavity as well as across the lens. The ribs 2922 arranged on the ledge 2920 can act as the stopping portion to support the medical instrument and enhance fluid flow against the ledge 2920 to be directed across the lens or distal end of medical instrument. The inner wall of the first lumen 2930 adjacent to the venting lumen 2932 can have the same diameter as the medical instrument to let gas flow across the lens and hold the medical instrument in position within the accessory. The venting lumen 2932 allows gas to be vented from the surgical cavity through the accessory 2900. The venting lumen 2932 has an inlet 2934 on the outer wall of the body of the accessory 2900 as shown in FIG. 29A. Gas can pass down the lumen and can be directed across the distal end of the medical instrument by the ledge 2920 as the gas exits into the surgical cavity as well as venting gas through the venting lumen 2932 as shown by the arrows in FIGS. 29A-29C. FIG. 29D, which is a cross-section through line 29D-29D of FIG. 29C, illustrates the arrangement of the ribs 2922 and the first lumen 2930 and second lumen 2932 in the accessory 2900 FIG. 29E, which is a cross-section through line 29E-29E of FIG. 29C, shows the ledge 2920 covering part of one side of the distal opening of the first lumen 2930 of the accessory 2900. It will be appreciated that in other embodiments, the accessory 2900 can include a plurality of venting lumens and more than one associated venting port.

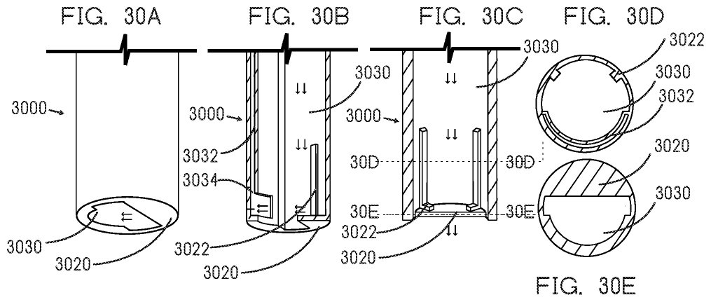

FIGS. 30A-30E illustrate a medical instrument accessory 3000 with deflection structure, configured as a ledge 3020, and a venting lumen 3032. The ledge 3020 is arranged to allow directing gas across a lens of a medical instrument arranged within the accessory 3000. The ledge 3020 can extend radially inward from the distal end of the primary or first lumen 3030 as shown in FIGS. 30A-30B. The medical instrument (not shown) can be positioned within the first lumen 3030. As shown in FIG. 30A-30B, the gas can be directed across the distal end of the accessory 3000 by the ledge 3020 and will pass over the distal end of the medical instrument. The venting lumen 3032 can vent gas from the distal end of the accessory 3000 after it has passed across the medical instrument. In some cases, the venting lumen inlet 3034 can be positioned on an inner wall of the lumen 3030 as shown in FIG. 30B. FIGS. 30B-30C illustrate partial cross-sectional embodiments of the accessory 3000. The accessory 3000 can also include one or more protruding ribs 3022 arranged to position the medical instrument within the accessory. The ribs 3022 can be arranged on the inner wall of the first lumen 3030 of the accessory 3000. The ribs can be arranged concentrically or substantially concentrically around the inner wall of the first lumen 3030 of the accessory 3000. The one or more protruding ribs 3022 and the ledge 3020 can direct the flow across the lens through the channel created by the protruding ribs 3022, the medical instrument (not shown), the inner wall of the accessory 3000, and the ledge 3020. The ribs 3022 can be concentric around the scope to let gas flow directly into the body cavity as well as across the lens. The protruding ribs 3022 arranged on the ledge 3020 can support the medical instrument during use and provide space for fluid flow around the instrument and against the ledge 3020 to be directed across the lens or distal end of the medical instrument. The inner wall of the first lumen 3030 adjacent to the venting lumen 3032 can have the same diameter as the medical instrument to let gas flow across the lens and hold the medical instrument in position within the accessory 3000. Gas can pass down the lumen and can be directed across the distal end of the medical instrument by the ledge 3020. The gas may then vent away from the distal end of the accessory 3000, after passing across the medical instrument, as shown by the arrows in FIGS. 30A-30C. FIG. 30D, which is a cross-section through line 30D-30D of FIG. 30C, illustrates arrangement of the ribs 3022 and the first lumen 3030 and second (venting) lumen 3032 in the accessory 3000. FIG. 30E, which is a cross-section through line 30E-30E of FIG. 30C, shows the ledge 3020 extending over part of one side of the distal opening of the first lumen 3030 of the accessory 3000.

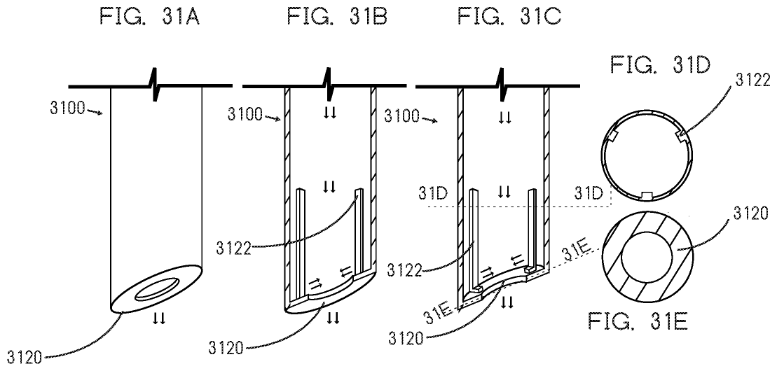

FIGS. 24A-30E illustrate accessories that can be used with medical instruments, such as scopes, that have a flat (0°) end, such as defined by or housing a lens, which is orthogonal to the longitudinal axis of the instrument. The accessories illustrated in FIGS. 31-37 can be used with medical instruments, such as scopes, that have an angled lens which is oblique to the longitudinal axis of the instrument. FIGS. 31A-37E illustrate a medical instrument accessory that is similar to the embodiments described with reference to FIGS. 24A-30E. However, the medical instrument accessory of FIGS. 31A-37E have an angled distal end relative to a longitudinal axis of the accessory, providing a geometry configured to provide the desired viewing angle(s). In some cases, the angled distal end can provide a viewing angle between about 1 degree and about 120 degrees. The angled distal end can be used to accommodate an angled medical instrument such as an angled scope and the embodiments described in FIGS. 31A-37E can be arranged to direct gas across the lens of the angled medical instrument.

FIGS. 31A-31E are similar to FIGS. 24A-24E and similar features are labeled similarly. Reference numerals of the same or substantially the same features share the same last two digits as those described in FIGS. 24A-24E. The deflection structure, in the form of an annular ledge 3120 in FIGS. 31A-31E, can be disposed at an angle, generally between 1 and 89°, to accommodate an angle-ended medical instrument, e.g. angled surgical scope, and direct fluid across the end in all directions of the ledge plane. Gas can pass down the lumen and can be directed across the distal end of the medical instrument by the ledge 3120 as the gas exits into the surgical cavity as shown by the arrows in FIGS. 31A-31C.

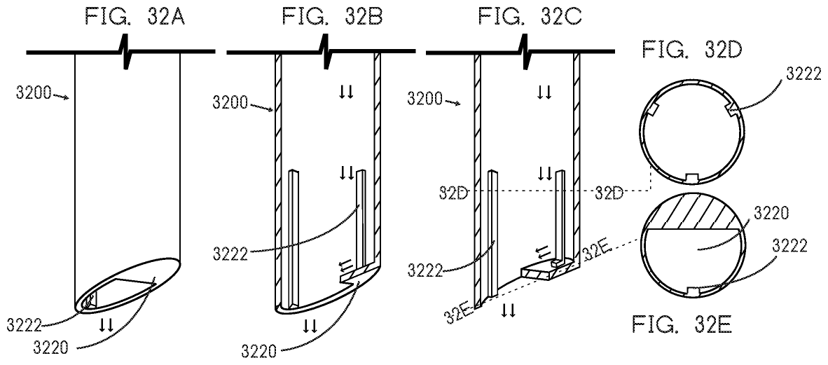

FIGS. 32A-32E are similar to FIGS. 25A-25E and similar features are labeled similarly. Reference numerals of the same or substantially the same features share the same last two digits as those described in FIGS. 25A-25E. The ledge 3220 on one side of the accessory 3200 in FIGS. 32A-32E can be angled to accommodate an angled medical instrument and direct gas across the lens in the ledge plane. Gas can pass down the lumen and can be directed across the distal end of the medical instrument by the ledge 3220 as the gas exits into the surgical cavity as shown by the arrows in FIGS. 32A-32C.

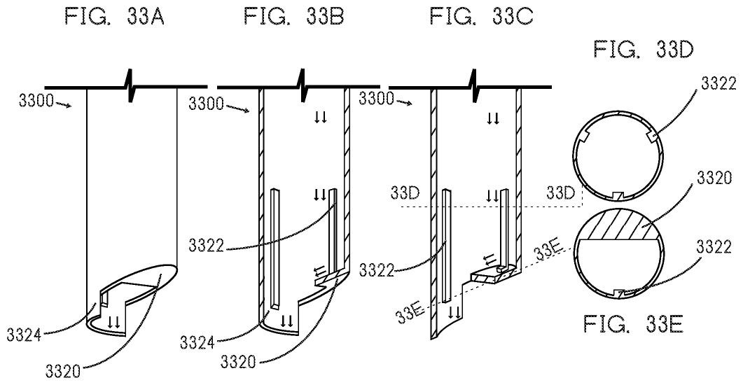

FIGS. 33A-33E are similar to FIGS. 26A-26E and similar features are labeled similarly. Reference numerals of the same or substantially the same features share the same last two digits as those described in FIGS. 26A-26E. The ledge 3320 on one side of the accessory in FIGS. 33A-33E can be angled to accommodate an angled medical instrument and direct gas across the lens. In some cases, the angled distal end can be positioned at an angle between about 1 degree and about 120 degrees. The flange 3324 is extended on the opposite side of the opening of the accessory 3300 from the ledge and can direct gas flow directly downward into the surgical cavity after it passes over the medical instrument. Gas can pass down the lumen and can be directed across the distal end of the medical instrument by the ledge 3320 and directed downwards into the surgical cavity by the flange 3320 as shown by the arrows in FIGS. 33A-33C.

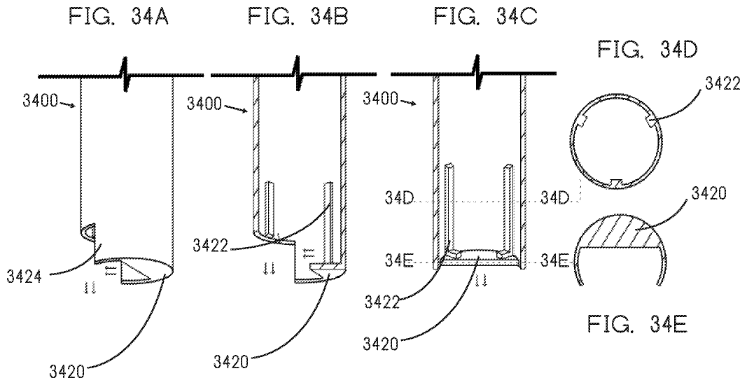

FIGS. 34A-34E are similar to FIGS. 27A-27E and similar features are labeled similarly. Reference numerals of the same or substantially the same features share the same last two digits as those described in FIGS. 27A-27E. The ledge 3420 on one side of the accessory in FIGS. 34A-34E can accommodate an angled medical instrument and direct gas across the lens. The flange 3424 is extended on the side with the ledge to create a cut-out on the opposite side of the ledge to reduce the chance of gas deflection.

FIGS. 35A-35E are similar to FIGS. 28A-28E and similar features are labeled similarly. Reference numerals of the same or substantially the same features share the same last two digits as those described in FIGS. 28A-28E. The distal end and the ledge 3520 on one side of the open end of the accessory in FIGS. 34A-34E can be angled to accommodate an angled medical instrument and direct gas across the lens. The second lumen 3532 in the medical instrument accessory 3500 can channel insufflation gas to the surgical cavity.

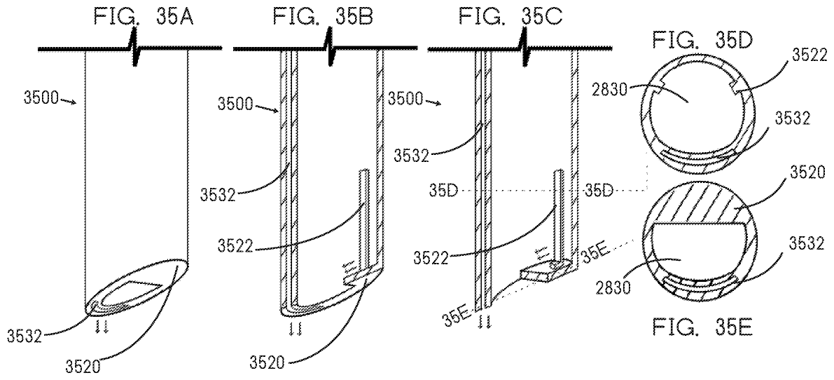

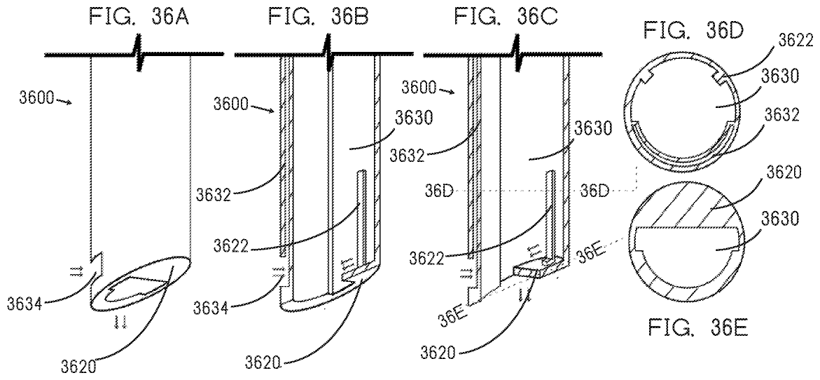

FIGS. 36A-36E are similar to FIGS. 29A-29E and similar features are labeled similarly. Reference numerals of the same or substantially the same features share the same last two digits as those described in FIGS. 29A-29E. The distal end and the ledge 3620 on one side of the accessory in FIGS. 36A-36E can be angled to accommodate an angled medical instrument and direct gas across the lens. The second lumen 3632 in the medical instrument accessory 3600 can vent gas out of the field of vision to remove any smoke out of the surgical cavity that may be causing optical problems.

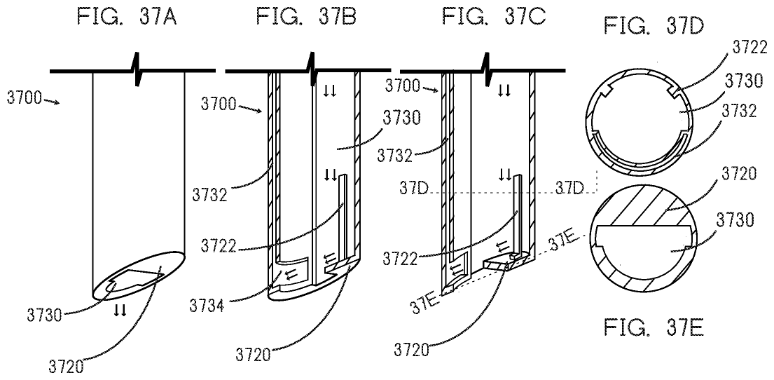

FIGS. 37A-37E are similar to FIGS. 30A-30E and similar features are labeled similarly. Reference numerals of the same or substantially the same features share the same last two digits as those described in FIGS. 30A-30E. The distal end and the ledge 3720 on one side of the accessory in FIGS. 37A-37E can be angled to accommodate an angled medical instrument and direct gas across the lens. The second lumen 3732 with an inlet on the inner wall of the medical instrument accessory 3700 can vent gas through the accessory after passing across the medical instrument. Gas can pass down the lumen and can be directed across the distal end of the medical instrument by the ledge 3720 and the gas can vent through the accessory after passing across the medical instrument as shown by the arrows in FIGS. 37A-37C.

### Examples of Heating of Medical Instrument Lens for use with a Medical Instrument Accessory

FIG. 38 illustrates a heating device 3840 that can be used with any of the medical instrument accessories described herein. Heating the medical instrument and/or fluids, for example gases, can help to prevent condensation by keeping the medical instrument lens temperature above the humidity dew point temperature of the gas surrounding the lens. This can be effective for condensation caused by the surgical cavity conditions and electrosurgery. As shown in FIG. 38, the heating device 3840 can be a coiled heating device that is incorporated into the walls of a medical instrument accessory. As shown in FIG. 38, a medical instrument 3810 can be received by the medical instrument accessory 3800. The accessory 3800 can have a body 3804 that can mount over or receive at least a portion of the medical instrument 3810. The body 3804 can have a lumen 3806 and an inner wall 3812. The inner wall 3812 can be spaced apart from a surface of the medical instrument 3810 when the device is in use by guide elements (not shown), for example, the guide elements described with respect to FIGS. 12A-37E, and may completely or only partially circumscribe the accessory 3800. The body 3804 can include a heating device 3840. In use, the heating device 3840 can directly or indirectly heat the medical instrument 3810. As shown in FIG. 38, the gas can enter the lumen 3806 of the body 3804 of the medical instrument accessory 3800 and be heated by the heating device 3840. The heated gas can pass over the distal end of the medical instrument 3810. In some cases, the heating device 3840 can provide heating along the length (or substantially along the length) of the shaft of the medical instrument 3810.

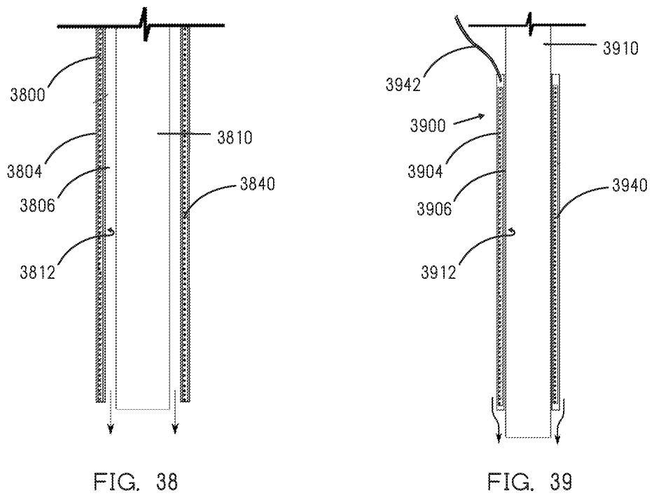

FIG. 39 illustrates a heating device 3940 that can be used with any of medical instrument accessories described herein. The heating device 3940 can be a coiled heating device that is incorporated into a medical instrument accessory 3900 and used to heat a medical instrument 3910 located in the accessory 3900. The medical instrument accessory 3900 can be used with a medical instrument 3910 and cannula (not shown) such as described herein. Additionally and/or alternatively, the heating device 3940 can be used to heat the medical instrument as well as gas that passes through the gap between the accessory and cannula. The accessory 3900 can have a body 3904 that can mount over at least a portion of the medical instrument 3910. The body 3904 can have a lumen 3906 and an inner wall 3912. The inner wall 3912 can contact a surface of the medical instrument 3910. The body 3904 can include or incorporate the heating device 3940. Heated gas can flow through a flow path defined by an outer wall of the accessory 3900 and a cannula lumen and then pass over the distal end of the medical instrument 3910 (as shown by arrows at the distal end). In some cases, the heating device 3940 can provide heating along the length (or substantially along the length) of the shaft of the medical instrument 3910. In some cases, the heating device 3940 can be powered by an electrical connection 3942 as shown in FIG. 39.

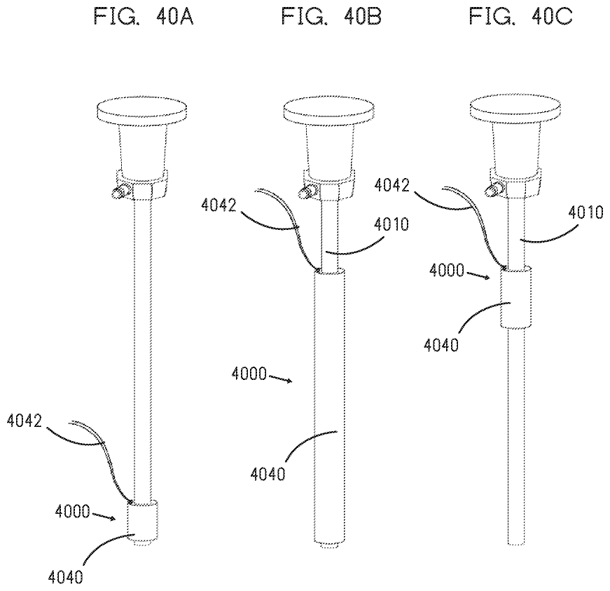

FIGS. 40A-40C illustrate a heating device 4040 that can be used with any of the medical instrument accessories described herein. The heating device 4040 can be incorporated into a medical instrument accessory 4000 used to heat the medical instrument 4010. The heating device 4040 can be located with respect to, or focused on a specific area of the medical instrument 4010. For example, FIG. 40A illustrates a heating device 4040 focused at the distal end of the medical instrument, such as the scope lens, and can heat the distal end more directly. The body of the medical instrument accessory 4000 can be various lengths. In some cases, the body can have a length that is less than, substantially equal to, or even longer than the length of the medical instrument shaft. FIG. 40B illustrates a heating device 4040 on a majority of the medical instrument 4010. FIG. 40C illustrates a heating device 4040 with the heating focused closer to the proximal end of the medical instrument 4010 to conduct heat down the medical instrument shaft.

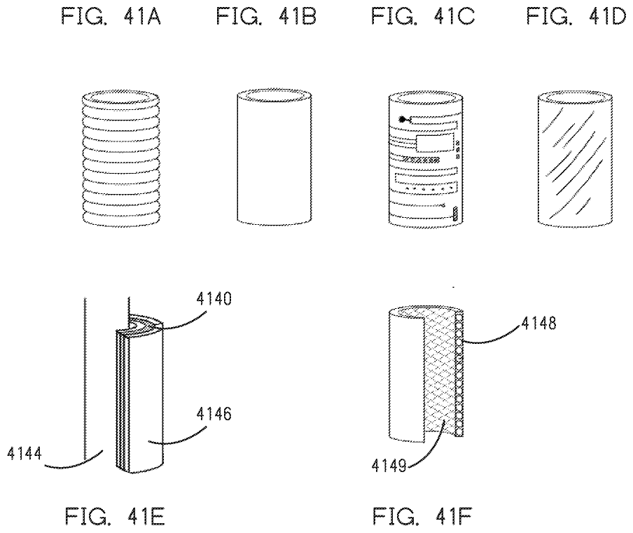

FIGS. 41A-41F illustrate various heating methods and materials which can be incorporated into any of the medical instrument accessory embodiments described herein. FIG. 41A illustrates a coil heating device. FIG. 41B illustrates a resistive material heating device. FIG. 41C illustrates a flexible PCB heating device. FIG. 41D illustrates a chemical reaction heating device. FIG. 41E illustrates a layered heating device with an insulated material. The layered heating device can include a protective inner layer of the wall 4144, a heating device 4140 in the middle layer, and an insulative protective material outer layer 4146. FIG. 41F illustrates a heating device that utilizes latent heat of vaporization. A desiccant 4148 can be used in the wall of the medical instrument accessory. This can convert the moisture of gas to liquid in an exothermic reaction giving off heat. A mesh 4149 can be used to expose the desiccant to gas flow in the medical instrument accessory.

FIGS. 42A-42F illustrate various options for providing power to the heating devices. These options include powering by an external unit (FIG. 42A); an associated cannula (FIG. 42B); a battery (FIG. 42C); a tubeset (FIG. 42D); a tube (FIG. 42E); and a wireless power transfer (FIGS. 42F-42G). The wireless power transfer can work with heating coils. FIG. 42G, which is a cross-section through line marked 42G-42G in FIG. 42F, illustrates the cross-section of a cannula 4250, medical instrument accessory heating device 4240, and a medical instrument 4210. The cannula 4250 can include a heated coil and the accessory heating device 4240 can include a coil. The cannula coil can transfer power to the heating device 4240.

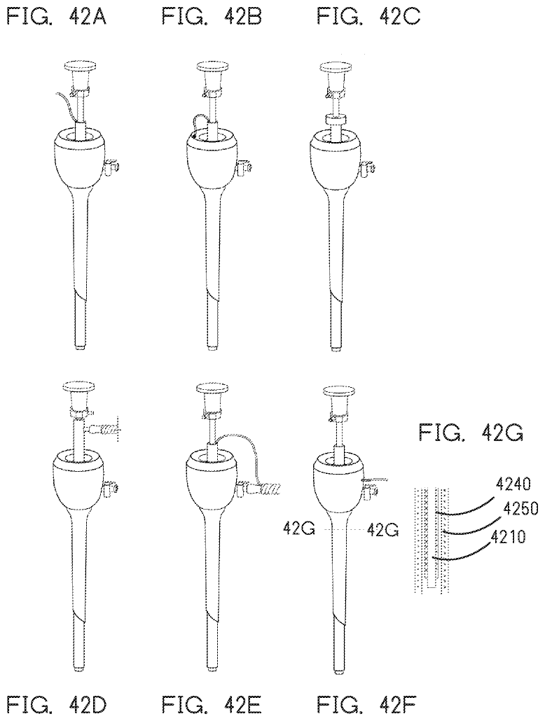

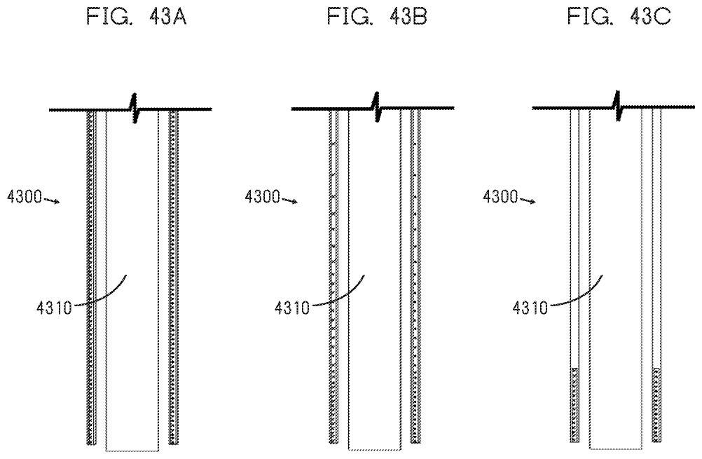

FIGS. 43A-43C illustrate example embodiments of how heat may be transferred to gas that flows by or through the medical instrument accessory 4300. FIG. 43A illustrates a medical instrument accessory 4300 with a heating device that provides a constant heating as indicated by the dotted pattern on the accessory wall. The heating device 4340 of FIG. 43B provides a gradient heating, indicated by the gradient dotted pattern on the accessory wall. The embodiment shown in FIG. 43C has a heating device 4340 that provides a localized heating as indicated by the dotted pattern on the distal end of the accessory wall. Although the heating in FIG. 43C is shown localized to the distal end of the medical instrument and accessory, the heating can be localized to any portion of the medical instrument shaft or accessory wall.

### Gas Supply Methods

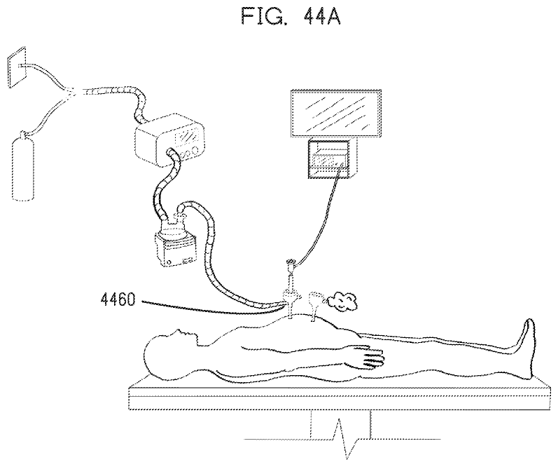

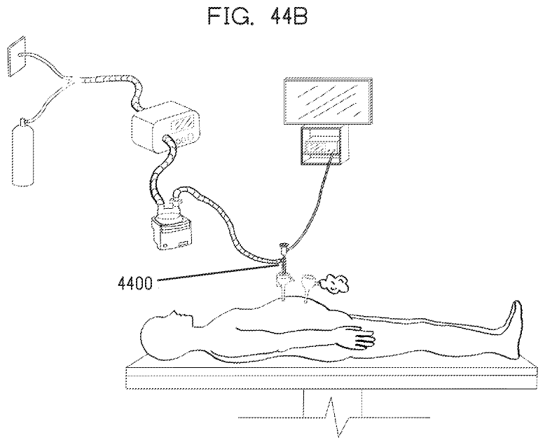

FIGS. 44-47 illustrate exemplar gas supply options that can be used with any of the cannulas, medical instrument accessories, or medical instruments described herein. FIG. 44A illustrates the use of insufflation gas delivered to the surgical cavity directly through the insufflating cannula 4460. A surgical humidifier as well as other types of humidification systems, may be placed between the insufflator and the cannula as illustrated in FIG. 44A. The insufflation gas can be delivered to the surgical cavity directly through the cannula.

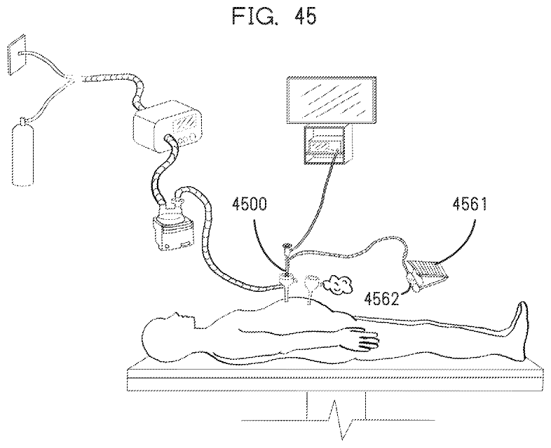

FIG. 44B illustrates delivery of insufflation gas (such as, for example, CO2 or other gases, or combinations thereof) to the surgical cavity directly through the medical instrument accessory 4400. A surgical humidifier, or any other type of suitable humidification system, may be placed between the insufflator and the cannula as illustrated in FIG. 45. The insufflation gas is delivered to the surgical cavity through the inlet in the medical instrument accessory 4400. The gas can be delivered through the inlet, into the lumen and out of the distal end of the medical instrument accessory located in the surgical cavity.

FIG. 45 illustrates the use of a gas canister 4562, for example, a CO2 canister that supplies a secondary gas source to the medical instrument accessory 4500. In some cases, although CO2 is described, the gas canister can supply other gases including but not limited to air, oxygen, nitrous oxide, argon, helium, and/or mixtures of these gases. The gas can be delivered to the medical instrument accessory 4500 when a control, for example, a foot pedal 4561 is pressed as illustrated in FIG. 45. In some cases, the control can be in operative communication with the gas flow mechanism, for example, a valve. Pressing the foot pedal can release a burst of gas, which can flow through the medical instrument accessory 4500 from the gas inlet, through the lumen and exiting the distal end of the medical instrument accessory to flush over the distal end of the medical instrument or lens.

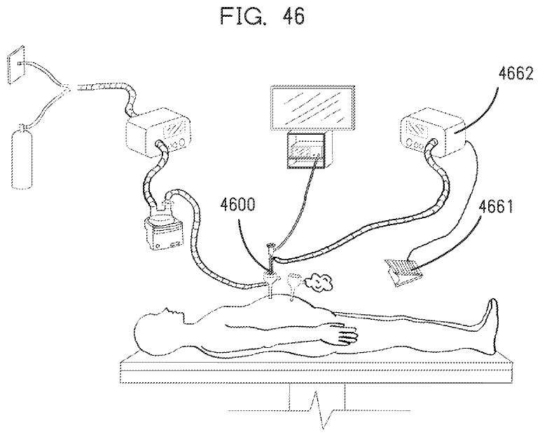

FIG. 46 illustrates the use of an additional CO2 gas supply unit, for example insufflator, 4662 that delivers CO2 gas direct to the medical instrument accessory 4600. The CO2 gas can be in operative communication with a gas control mechanism, such as a foot pedal 4661. The CO2 can be delivered to the medical instrument accessory 4600 when the foot pedal 4661 is pressed as illustrated in FIG. 46.

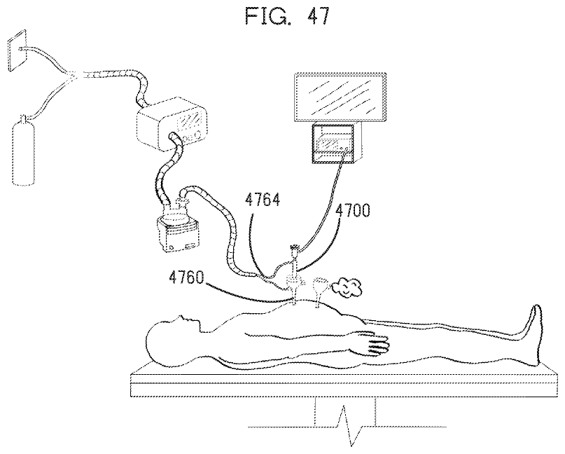

FIG. 47 illustrates delivery of insufflation gas to the surgical cavity with a split 4764 between the medical instrument accessory 4700 and the cannula 4760. The insufflation gas can be delivered to the surgical cavity through both the cannula 4760 and the medical instrument accessory 4700 with a split connection 4764 at the distal end of the insufflation tube as shown in FIG. 47.

### Attachment Options

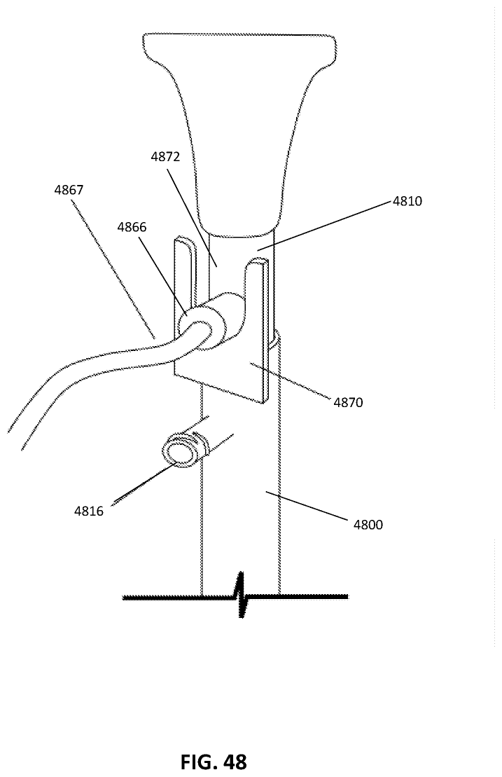

FIGS. 48-52 illustrate example attachment options for securement of components of the surgical assembly that incorporates a medical instrument accessory described herein and that can be used for any of the cannulas, medical instrument accessories, or medical instruments described herein.

FIG. 48 illustrates an embodiment of an alignment feature 4870 which is releasably securable to a medical instrument accessory 4800. It will be appreciated that in other embodiments, the feature 4870 is integrally formed with, or permanently affixed, to the accessory 4800. Fluid, such as insufflation gases, can be delivered directly to the medical instrument accessory 4800 through a fluid inlet 4816. The alignment feature 4870 can aid correct orientation of the medical instrument accessory 4800 relative to the medical instrument 4810, such as a scope. The alignment feature 4870 defines a recess 4872 dimensioned to partially surround a connection port 4866 protruding from the medical instrument 4810, such as a light cable connector. The recess 4872 of the alignment feature 4870 can be used to inhibit relative rotational and axial movement of the medical instrument 4810 relative to the medical instrument accessory 4800. For example, this may assist with arranging the distal end and lens of the instrument 4810 relative to the distal end of the accessory 4800 to enhance directing fluid flow across the end and lens of the instrument 4810 by the accessory 4800. Furthermore, the alignment feature 4870 may arrange the fluid inlet 4816 adjacent the connection port 4866 to enhance convenience of connecting the light source and fluid source to the instrument 4810 and accessory 4800, respectively. In other embodiments, the recess of the alignment feature 4870 can be defined by alternatively shaped structures, such as a V-shape defined by two elongate members extending in different directions from a common location.

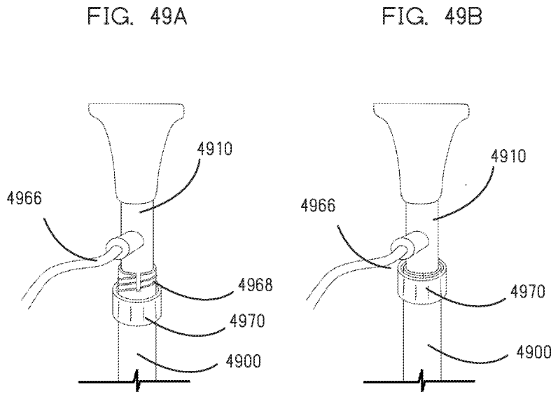

FIGS. 49A-49B illustrate an embodiment of an accessory securement assembly, including a threaded ring 4970 configured to engage with a proximal end of the accessory. In some cases, the gas can be delivered to a cannula and the gas can be redirected by the scope accessory. The accessory can include a threaded portion 4968 on the proximal end of the shaft, corresponding to threaded features (not shown) located on an inner surface of the threaded ring 4970. FIG. 49A illustrates the ring 4970 removed from the threaded features 4968 of the accessory 4910. FIG. 49B illustrates the ring 4970 in engagement with the accessory when the threaded surface of the ring 4970 is mated with the corresponding threaded portion of the accessory. In some cases, the medical instrument accessory can have a tapered proximal end. The ring 4970 can tighten the tapered end of the medical instrument accessory around the medical instrument 4910. In some cases, instead of or in addition to the threaded engagement, a friction fit connection on the tapered end of the connector portion could be used.

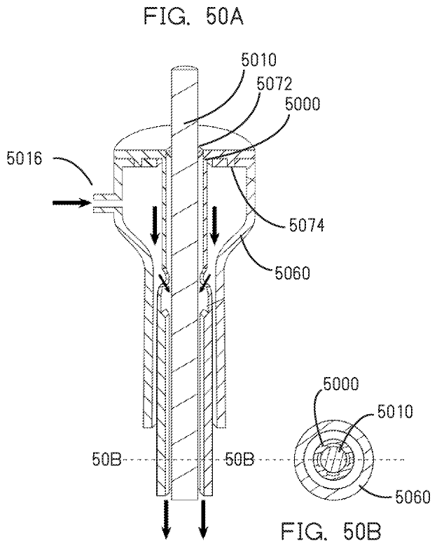

FIGS. 50A-50B illustrate an embodiment of an attachment assembly to attach the accessory to a trocar assembly or cannula. The medical instrument accessory 5000 can be attached inside a cannula 5060 and secured in place by clipping into the cannula housing or by any other suitable means. The medical instrument 5010 can move freely relative to the accessory 5000 and cannula 5060 assembly. The insufflation gas can be supplied to the cannula through the gas inlet 5016 and redirected through the medical instrument accessory 5000 by any of the means described herein. The proximal end of the accessory 5000 can have a seal 5072 to prevent gas from escaping. The medical instrument accessory 5000 can include protrusion(s) that engage with corresponding apertures in cannula body. The medical instrument accessory 5000 can be secured to the cannula by a clip press mechanism 5074 that clips into the proximal end of the cannula. FIG. 50B illustrates a cross-sectional view through line 50B-50B in FIG. 50A. As illustrated in FIG. 50A, the gas flow can enter the gas inlet 5016 and can pass down the medical instrument 5010 between the ribs or any other features that directs gas flow in the cannula or medical instrument accessory as described herein.

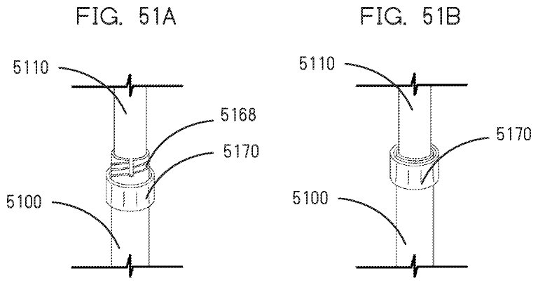

FIGS. 51A-51B illustrate a medical instrument accessory 5100 with a threaded ring 5170 to align and attach the medical instrument accessory 5100 on the medical instrument 5110. The medical instrument accessory 5100 can include a threaded ring 5170 that tightens over the tapered end of the medical instrument accessory 5100. The medical instrument accessory 5100 can have a shaft with male threaded features 5168 that correspond to the threading in the threaded ring 5170. The ring can be tightened and closed onto the medical instrument 5110 when the ring 5170 is screwed up the shaft of the accessory 5100. This allows the accessory 5100 to attach directly to the medical instrument 5110. For example, the accessory 5100 can attach to a medical instrument, such as a cutting tool. In some cases, instead of or in addition to the threaded engagement, a friction fit engagement on the tapered end of the connector portion could be used. FIG. 51A illustrates the ring 5170 in an open configuration and removed from the threaded features 5168 of the medical instrument 5110. FIG. 51B illustrated the ring 5170 in a closed configuration with the ring 5170 screwed up the shaft of the medical instrument accessory 5100.

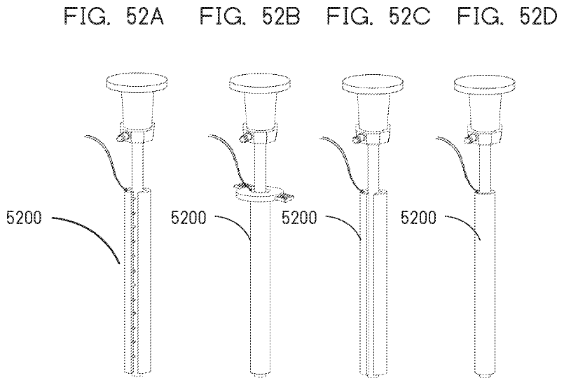

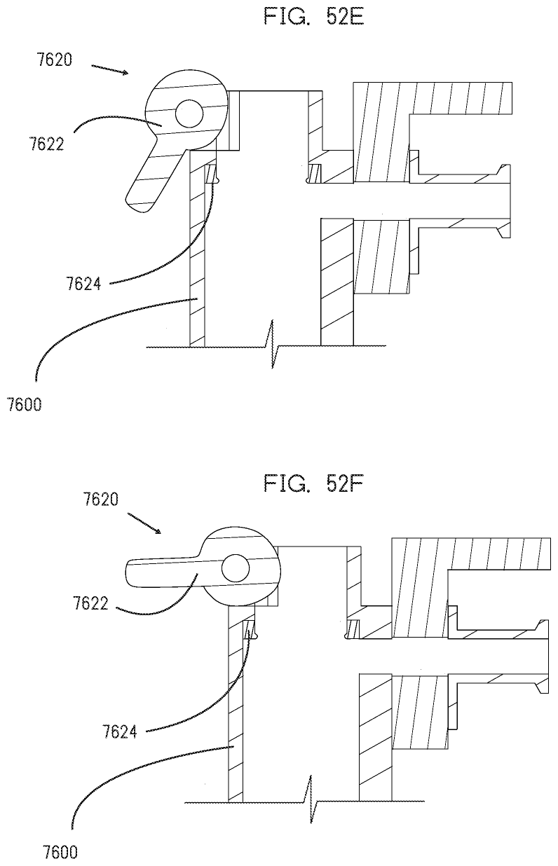

FIGS. 52A-52D illustrate example attachment assemblies for securing any of the aforementioned medical instrument accessories, particularly those incorporating a heating device. FIG. 52A illustrates the use of an attachment that is a clip on two sections of the heating device. FIG. 52B illustrates the use of an attachment that uses a push and release clamp for the heating device. FIG. 52C illustrates the use of an attachment that uses an adhesive on two sections of the heating device. FIG. 52D illustrates the use of an attachment that uses friction fit for attachment of the heating device.

FIGS. 52E-52F illustrates a locking mechanism 7620 arranged adjacent an opening defined at a proximal end of a medical instrument accessory 7600. The locking mechanism 7620 includes a cam 7622 which is rotatable about an axis to move between a first (open) position (shown in FIG. 52E) and second (locking) position (shown in FIG. 52F). The locking mechanism 7620 can be operated, for example, by a user operating a lever 7626 extending from the cam 7622. As shown in FIG. 52E, the cam 7622 is shown in the open position so that the cam 7622 does not obstruct the opening and, consequently, does not interact with a medical instrument arranged within the accessory 7600. FIG. 52F illustrates the cam 7622 in the locked position to extend into the opening. Arranging the cam 7622 in the locked position allows the cam 7622 to interfere with the medical instrument to secure the instrument in the accessory 7600. The accessory 7600 can also include a seal element 7624 similar to the seal element 7520 described with reference to FIG. 75 below.

### Examples of Medical Instrument Attachment to Direct Gas Flow

In some cases, the medical instrument accessory can include an attachment structure/framework configured to attached to the medical instrument. FIGS. 53A-53B illustrate a cannula 5360 with a gas flow path between the cannula lumen 5306 and the medical instrument 5310. The cannula 5360 can include a gas inlet 5316. The medical instrument attachment 5300 can have a body 5304 and a structure to position a medical instrument 5310 in the cannula lumen 5306. The medical instrument attachment 5300 can be an attachment positionable on the medical instrument 5310 as shown in FIG. 53A. The medical instrument attachment that attaches to the medical instrument 5310 can incorporate guide elements as described herein. In some cases, the attachment 5300 can include one or more ribs 5320 as shown in FIG. 53A. In other embodiments, the medical instrument attachment 5300 can include any of the features described herein to direct gas flow through the cannula. The ribs 5320 can position the medical instrument shaft in the cannula lumen 5360 such that a gas flow path is defined between the lumen 5360 wall and the shaft of the medical instrument 5310. The gas flow path can direct gas around a distal end of the medical instrument 5310. The ribs 5320 can lie on or adjacent an outer surface of the medical instrument 5310. Gas can enter the cannula 5360 through the inlet 5316 and travel in a gas flow path created between the cannula inner sidewall 5312 and the medical instrument 5310 and medical instrument attachment 5300 assembly. FIG. 53B illustrate a cross-sectional view through line 53B-53B in FIG. 53A. As illustrated in FIG. 53B, the gas can be channeled between the medical instrument 5310 and the lumen 5306 of the cannula 5360.

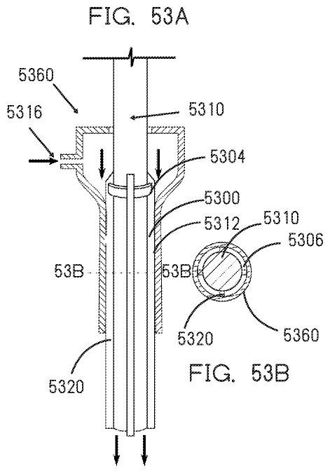

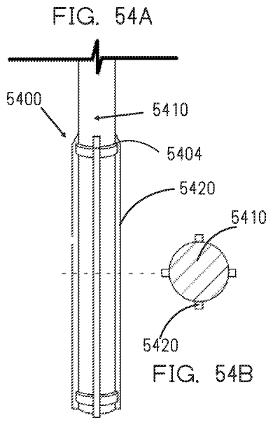

FIGS. 54A and 54B illustrate a medical instrument 5410 with a medical instrument attachment 5400. FIG. 54A-54B illustrate the medical instrument attachment 5400 is similar to the medical instrument attachment 5400 describe with reference to FIGS. 53A-53B. However, the assembly is shown without the cannula surrounding it. The medical instrument attachment 5400 can have a body 5404 and ribs 5420 that position the medical instrument shaft in a cannula lumen or other medical instrument accessory. The ribs 5420 can lie on or adjacent an outer surface of the medical instrument 5410. In some cases, the body 5404 can be secured to the medical instrument 5410. The ribs 5420 on the medical instrument 5410 can be any length along the shaft on the medical instrument 5410. FIG. 54B illustrate a cross-sectional view through line 54B-54B in FIG. 54A. As illustrated in FIG. 54B, the ribs 5420 can hold the medical instrument 5410 concentrically in a cannula and gas can flow concentrically around the medical instrument.

FIGS. 55A-55B illustrate a medical instrument 5510 with a pair of medical instrument attachments 5500 attached to the medical instrument 5510. In some cases, one or more medical instrument attachments 5500 can be attached to the medical instrument 5510. The medical instrument attachment 5500 can have ribs 5520 that position the medical instrument shaft in a cannula lumen or other medical instrument attachments. The ribs 5520 can lie on or adjacent an outer surface of the medical instrument 5510. FIG. 55B illustrate a cross-sectional view through line 55B-55B in FIG. 55A. As illustrated in FIG. 55B, the ribs 5520 can be fit onto the medical instrument 5510 and can hold the medical instrument 5510 concentrically in a cannula and gas can flow concentrically around the medical instrument. In some cases, the medical instrument attachment can be fit onto the medical instrument 5510 by a friction fit or adhesive.

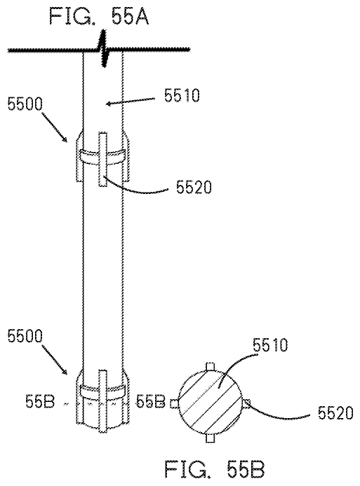

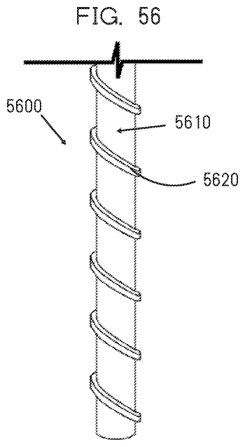

FIG. 56 illustrates a medical instrument 5610 with a medical instrument attachment 5600 attached to the medical instrument 5610. The medical instrument attachment 5600 can be a spiral attachment 5620 that is secured on to the medical instrument 5610 to guide flow around the medial instrument 5610 in a vortex pattern. In some embodiments, the gas flow can be guided between the medical instrument and a cannula lumen.

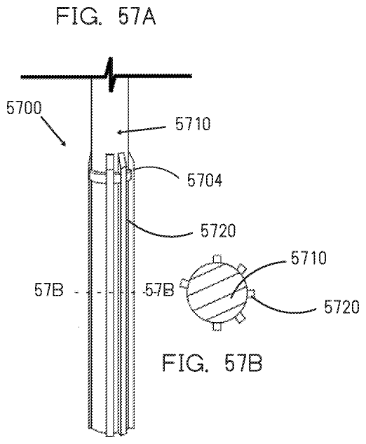

FIGS. 57A-57B illustrate a medical instrument 5710 with a medical instrument attachment 5700. The medical instrument attachment 5700 can have a body 5704 and ribs 5720 that position the medical instrument shaft in a cannula lumen or other medical instrument attachment. The ribs 5720 can lie on or adjacent an outer surface of the medical instrument 5710. In some cases, the body 5704 can be secured to the medical instrument 5710. As shown in FIG. 57A, the ribs 5720 can be unevenly spaced around the medical instrument 5710 to deflect contamination or water droplets away from the lens or encourage evaporation of condensation/fogging that has formed on the lens. The ribs can be at any point along the accessory or the full length. FIG. 57B illustrates a cross-sectional view through line 57B-57B in FIG. 57A. FIG. 57B illustrates the uneven spacing of the ribs 5720 on the medical instrument 5710. The ribs can hold the medical instrument concentrically in a cannula and gas can flow concentrically around it.

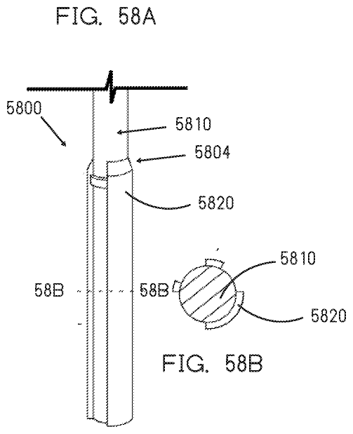

FIGS. 58A-58B illustrate a medical instrument 5810 with a medical instrument attachment 5800. The medical instrument attachment 5800 can have a body 5804 and ribs 5820 that position the medical instrument shaft in a cannula lumen or other medical instrument attachment. The ribs 5820 can lie on or adjacent an outer surface of the medical instrument 5810. In some cases, the body 5804 can be secured to the medical instrument 5780. As shown in FIG. 58A, the ribs 5820 can be unevenly widths around the medical instrument 5810 to entrain gas flow over the lens as a cleaning effect. The ribs can be at any point along the attachment or the full length. FIG. 58B illustrate a cross-sectional view through line 58B-58B in FIG. 58A. FIG. 58B illustrates the uneven widths of the ribs 5820 on the medical instrument 5810. The ribs can hold the medical instrument concentrically in a cannula and gas can flow concentrically around it.

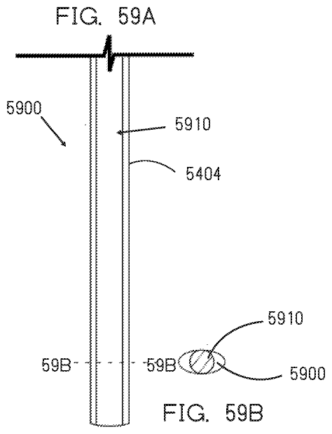

FIGS. 59A-59B illustrate a medical instrument 5910 with a medical instrument accessory 5900. The medical instrument accessory 5900 can have a body 5904 and a non-circular cross-section. As shown in FIG. 59A, the medical instrument accessory 5900 can be secured to the medical instrument 5910. Depending on the medical instrument 5910 positioning the gas can either flow concentrically around the medical instrument 5910 or be entrained over it. FIG. 59B illustrate a cross-sectional view through line 59B-59B in FIG. 59A. FIG. 59B illustrates the non-circular shape of the medical instrument accessory 5900. Although the shape of the non-circular cross-section is shown as an oval shape, the non-circular cross-section of the body of the medical instrument accessory can be any non-circular shape.

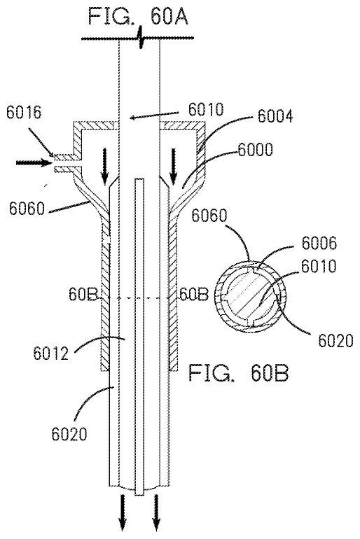

In some cases, the medical instrument can have features integrated with, that is, built into the medical instrument itself. FIGS. 60A-60B illustrate a cannula 6060 with a gas flow path between the cannula lumen 6006 and the medical instrument 6010. The cannula 6060 can include a gas inlet 6016. The medical instrument 6010 can have a shaft 6012 and a protrusion 6020 to position a medical instrument 6010 in a cannula lumen 6006. In some cases, the surface of the shaft can have one or more protrusions extending radially outward. The one or more protrusions can come into contact with an inner wall of the cannula when in use. The protrusion 6020 can be built into the medical instrument 6010 as shown in FIG. 60A. The cannula inner wall and the protrusions can define a gas flow path. In some cases, the protrusion on the medical instrument 6010 can be one or more ribs 6020 as shown in FIG. 60A. In other embodiments, the protrusion can be any of the features described herein to direct gas flow through the cannula. The ribs 6020 can extend at least part of the length of the shaft 6012. The ribs 6020 on the medical instrument shaft 6012 can extend the length (or substantially the length) of the shaft. The gas flow path can be defined between the cannula 6060 and the shaft 6012 of the medical instrument 6010. Gas can enter the cannula 6060 through the inlet 6016 and travel in a gas flow path created between the cannula 6060 and the medical instrument 6010. The medical instrument 6010 can direct gas flow over or adjacent to a distal end of the shaft 6012. FIG. 60B illustrate a cross-sectional view through line 60B-60B in FIG. 60A. As illustrated in FIG. 60B, the gas can be channeled between the medical instrument 6010 and the lumen 6006 of the cannula 6060.

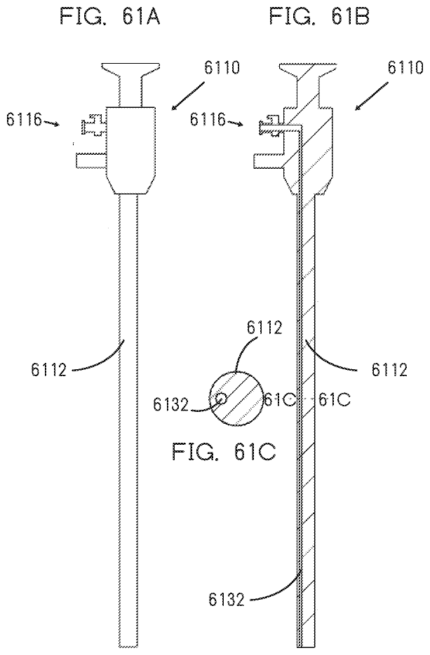

FIGS. 61A-61C illustrate an embodiment of a medical instrument with a lumen to direct gas flow through the medical instrument. FIG. 61A illustrates a medical instrument 6110 with a gas inlet 6116 and a shaft 6112. FIG. 61B illustrates a vertical cross-sectional view of a medical instrument 6110 with a shaft 6112 and an internal lumen 6132. As illustrated in FIG. 61B, the shaft 6112 can have a lumen 6132 that directs gas flow through the shaft 6112 and out of a distal end of the shaft 6112. The directed lumen 6132 can direct flow of gas across the distal end of the medical instrument or the lens. FIG. 61C illustrate a cross-sectional view through line 61C-61C in FIG. 61B. As illustrated in FIG. 61C, the lumen 6132 can be offset from the center of the shaft 6112.

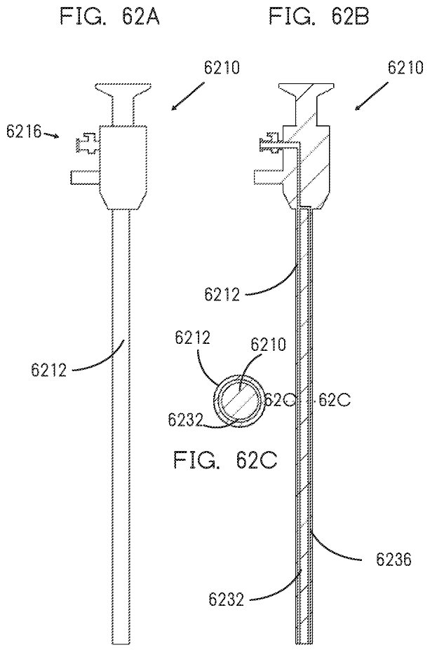

FIGS. 62A-62C illustrate an embodiment of a medical instrument with a lumen to direct gas flow through the medical instrument. FIG. 62A illustrates a medical instrument 6210 with a gas inlet 6216 and a shaft 6212. FIG. 61B illustrates a vertical cross-sectional view of a medical instrument 6210 with a shaft 6212 and an internal lumen 6232. As illustrated in FIG. 62B, the shaft 6212 can have a lumen 6232 that directs gas flow through the shaft 6212 and out of a distal end of the shaft 6212. The lumen 6232 can direct flow of gas for a concentric flow or flow across the distal end of the medical instrument or the lens. FIG. 62C illustrate a cross-sectional view through line 62C-62C in FIG. 62B. As illustrated in FIG. 62C, the lumen 6232 can be a concentric lumen for a directed flow around the distal end of the medical instrument.

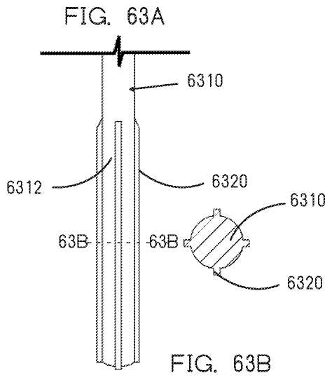

FIGS. 63A-63B illustrate protrusions arranged uniformly around the circumference of the shaft of the medical instrument to direct flow concentrically around the medical instrument. The protrusions can be ribs 6320 as illustrated in FIG. 63A. The ribs 6320 on the medical instrument can be any length along the medical instrument shaft 6312. The ribs 6320 can be arranged uniformly (or substantially uniformly) around the circumference of the shaft 6312. FIG. 63B illustrate a cross-sectional view through line 63B-63B in FIG. 63A. As illustrated in FIG. 63B, the ribs 6320 can hold the medical instrument 6310 concentrically in a cannula (not shown) and gas can flow concentrically around it.

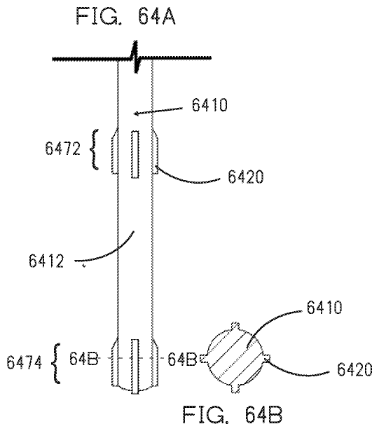

FIGS. 64A-64B illustrate two sets of protrusions arranged uniformly around the circumference of the shaft of the medical instrument to direct flow concentrically around the medical instrument. The first set of protrusions 6472 is horizontally offset from the second set of protrusions 6474. The first set of protrusions 6472 can be positioned at a proximal end of the shaft 6412 and the second set of protrusions 6474 can be positioned at a distal end of the shaft 6412 as illustrated in FIG. 64A. The sets of protrusions can be arranged anywhere along the shaft of the medical instrument. In some cases, the protrusions can be ribs 6420 as illustrated in FIG. 64A. The ribs 6420 on the medical instrument can be any length along the medical instrument shaft 6412 and positioned at any location. The ribs 6420 can be arranged uniformly (or substantially uniformly) around the circumference of the shaft 6412. FIG. 64B illustrates a cross-sectional view through line 64B-64B in FIG. 64A. As illustrated in FIG. 64B, the ribs 6420 can hold the medical instrument 6410 concentrically in a cannula (not shown) and gas can flow concentrically around it.

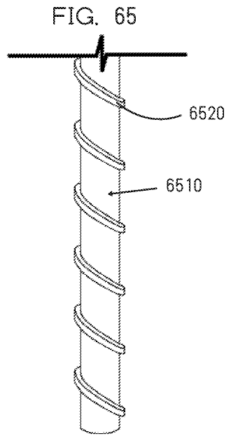

FIG. 65 illustrates protrusions in a spiral arrangement around the circumference of the shaft of the medical instrument to direct flow concentrically around the medical instrument. The protrusions can be a spiral fin 6520 as illustrated in FIG. 65. The spiral fin 6520 can direct flow in a vortex pattern around the medical instrument 6510. The gas flow can be guided between the medical instrument and a cannula (not shown).

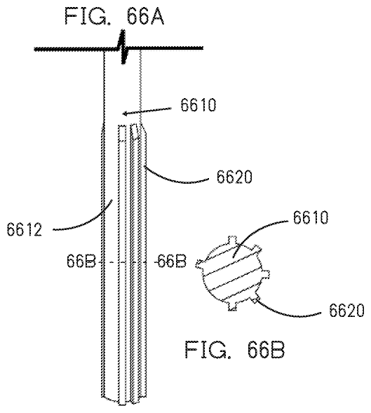

FIGS. 66A-66B illustrate protrusions arranged non-uniformly around the circumference of the shaft of the medical instrument to direct flow concentrically around the medical instrument. The protrusions can be unevenly spaced or non-uniformly arranged around the shaft of the medical instrument. In some cases, the protrusions can be ribs 6620 unevenly spaced around the medical instrument 6610 as illustrated in FIG. 66A-66B. The ribs 6620 on the medical instrument 6610 can be any length along the medical instrument shaft 6612 and positioned at any location. The ribs 6620 can be unevenly spaced around the medical instrument 6610 to deflect contamination or water droplets away from the lens or encourage evaporation of condensation/fogging that has formed on the lens. The ribs 6620 can be at any point along the shaft 6612 of the medical instrument 6610 or along the full length of the medical instrument 6610. FIG. 66B illustrate a cross-sectional view through line 66B-66B in FIG. 66A. As illustrated in FIG. 66B, the ribs 6620 can hold the medical instrument 6610 concentrically in a cannula (not shown) and gas can flow concentrically around it.

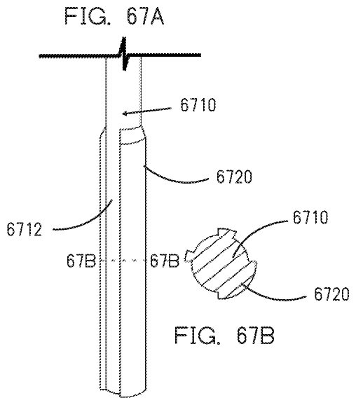

FIGS. 67A-67B illustrate protrusions with uneven widths around the shaft of the medical instrument to direct flow concentrically around the medical instrument. In some cases, the protrusions can be ribs 6720 with uneven widths around the medical instrument 6710 as illustrated in FIG. 67A-67B. The ribs 6720 on the medical instrument 6610 can be any length along the medical instrument shaft 6712 and positioned at any location. The ribs 6720 can be different widths around the medical instrument 6710 to deflect contamination or water droplets away from the lens or encourage evaporation of condensation/fogging that has formed on the lens. The ribs 6720 can be at any point along the shaft 6712 of the medical instrument 6710 or along the full length of the medical instrument 6710. FIG. 67B illustrate a cross-sectional view through line 67B-67B in FIG. 67A. As illustrated in FIG. 67B, the ribs 6720 can have uneven widths and can hold the medical instrument 6710 concentrically in a cannula (not shown) and gas can flow concentrically around it.

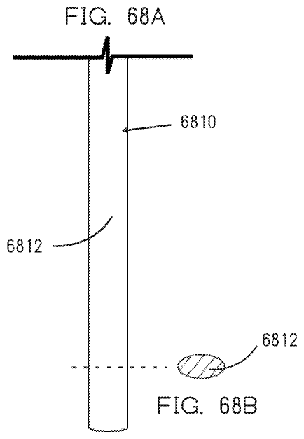

FIGS. 68A-68B illustrate a medical instrument 6810 with a shaft 6812 with a non-circular cross-section. The medical instrument 6800 can be used with a cannula lumen that is circular so no matter the positioning of the medical instrument gas can flow through the cannula. Depending on the medical instrument 6800 positioning the gas will flow around the scope or be entrained over it. FIG. 68B illustrate a cross-sectional view through line 68B-68B in FIG. 68B. As illustrated in FIG. 68B, the shaft 6812 can have a non-circular shape. In some case, the non-circular shape can be an oval shape as shown in FIG. 68B. In other cases, the shaft 6812 can be any non-circular shape.

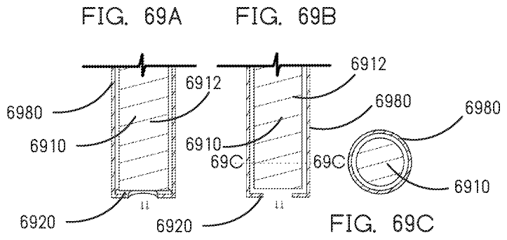

FIGS. 69A-69C illustrate a medical instrument 6910 including a deflection structure, in the form of a ledge 6920. The ledge 6920 extends inward from the edge of the distal end of the shaft 6912 of the medical instrument 6910. In some cases, the ledge 6920 can be an at least partially annular structure. In this embodiment, the ledge 6920 is configured as a continuous ring. However, it will be appreciated that the ledge 6920 may comprise a plurality of structures to form a discontinuous ring. The ring-shaped ledge 6920 can be positioned around the lens of the medical instrument to direct gas across the lens in all directions of the ledge plane. FIGS. 69A-69B illustrate the ring ledge 6920 around the distal end of the medical instrument 6910 to direct gas across the distal end of the medical instrument. FIG. 69C illustrates a cross-sectional view through line 69C-69C in FIG. 69B. As illustrated in FIG. 69C, the outer shell 6980 of the medical instrument 6910 can form a lumen for gas to flow around the central medical instrument 6910.

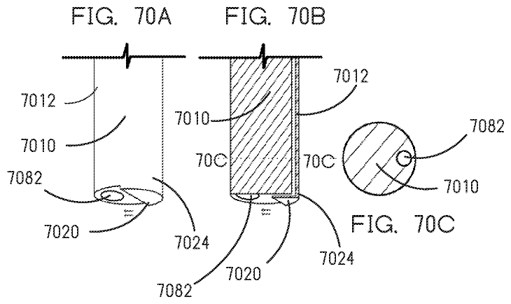

FIGS. 70A-70C illustrate a medical instrument 7010 with a deflection structure, in the form of a ledge 7020 on one side of the medical instrument 7010 to direct gas across the lens. The ledge 7020 extends inward from the edge of the distal end of the shaft 7012 of the medical instrument 7010. The medical instrument 7010 can also have a flange 7024 extending distally from the medical instrument. The flange 7024 can be adjacent to the lens 7082 of the medical instrument 7010. FIG. 70B illustrates a vertical cross-sectional view of the medical instrument 7010. As shown in FIGS. 70A-70B the ledge can direct gas flow across the lens of the scope. FIG. 70C illustrates a cross-sectional view through line 70C-70C in FIG. 70B. In some cases, the lumen inside the scope can deliver gas across the lens 7082. In some cases, the lumen can be a single lumen or a full concentric lumen. In some cases, the flange can direct gas flow from the concentric lumen across the distal end of the shaft.

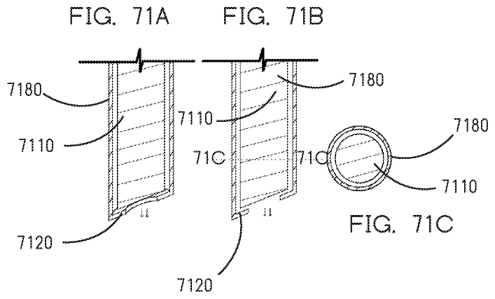

FIGS. 71A-71C illustrate a medical instrument 7110 having an outer body 7180 defining a circular ledge 7120 around an angled distal end. The outer body is spaced from an inner body to define a cavity between the bodies. Gas can be introduced between the bodies to cause the gas to be directed by the ledge 7120 across the angled end in all directions of the ledge plane. The ledge 7120 can extend inwardly from the edge of the distal end of the medical instrument 7110. As shown in FIGS. 71A-71B the ledge can direct gas flow across a lens at the distal end of the medical instrument. FIG. 71C illustrate a cross-sectional view through line 71C-71C in FIG. 71B. As illustrated in FIG. 71C, the outer body 7180 of the medical instrument 7110 can form a lumen for gas to flow around the inner body. In some cases, the lumen inside the medical instrument 7110 can deliver gas across the lens. In some cases, the lumen can be a single lumen or a full concentric lumen. In some cases, the ledge 7120 can direct gas flow from the concentric lumen across the distal end of the shaft.

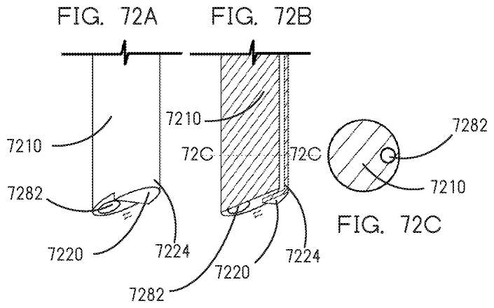

FIGS. 72A-72C illustrate an angled medical instrument 7210 with a deflection structure in the form of a ledge 7220 on one side of the angled medical instrument 7210 to direct gas across the lens. The ledge 7220 extends inward from the edge of the distal end of the medical instrument 7210. The medical instrument 7210 can also have a flange 7224 extending distally from the medical instrument. The flange 7224 can be adjacent to the lens 7282 of the medical instrument 7210. FIG. 72B illustrates a vertical cross-sectional view of the medical instrument 7210. As shown in FIGS. 72A-72B the ledge can direct gas flow across the lens of the scope. FIG. 72C illustrate a cross-sectional view through line 72C-72C in FIG. 72B. In some cases, the lumen inside the scope can deliver gas across the lens 7282. In some cases, the lumen can be a single lumen or a full concentric lumen. In some cases, the flange can direct gas flow from the concentric lumen across the distal end of the shaft.

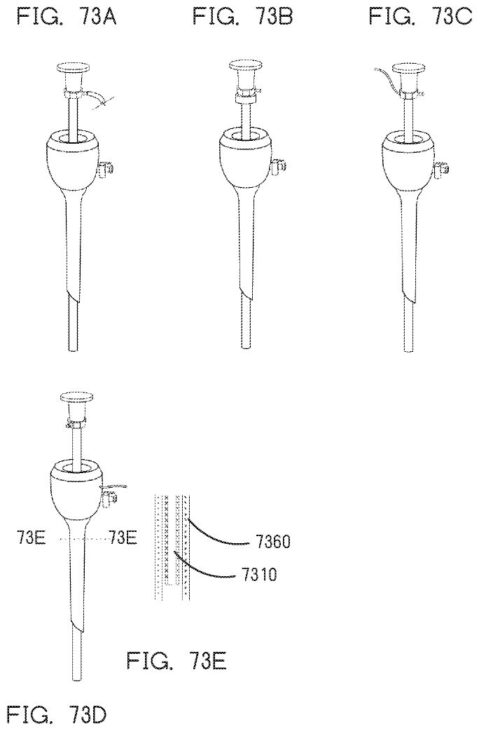

In some cases, the medical instrument or the shaft of the medical instrument itself can include a heating device. The heated medical instrument can be incorporated into any medical instrument embodiments described herein. FIGS. 73A-73E illustrate embodiments of power options for a heating device for a medical instrument. FIG. 73A illustrates a medical instrument powered directly by a cable. FIG. 73B illustrates a medical instrument powered directly by a battery. FIG. 73C illustrates a medical instrument powered externally. FIG. 73D illustrates a medical instrument powered directly by a wireless power transfer. In some cases, the wireless power transfer can be used with coils. FIG. 73E illustrate a cross-sectional view through line 73E-73E in FIG. 73D. As shown in FIG. 73E, there can be a coil in the cannula 7360 and a coil in the medical instrument 7310. The cannula coil can transfer power to the scope coil.

In some cases, the cannulas and accessories described herein can be based on manipulating the conditioned gas supply. In some cases, a continuously positive and/or substantially constantly flowing gas supply can be conditioned and manipulated, as well as intermittent and/or fluctuating flows. In some cases, optimizing the humidity source and manipulating the conditioned gas supply can assist in the use of the system and devices described herein. In some cases, continuous venting can enable continuous gas flow as well as conditioning or manipulating the intermittent and/or fluctuating flows. In some cases, the vented gas can be filtered.

The redirection of flow can increase resistance in the system so a reduced gas restriction would help to offset this and may improve compatibility with the gas supply. The reduced restriction at the gas connection, a tubeset with less friction, a tubeset with consistent diameter, and a tubeset with multiple connections can be used.

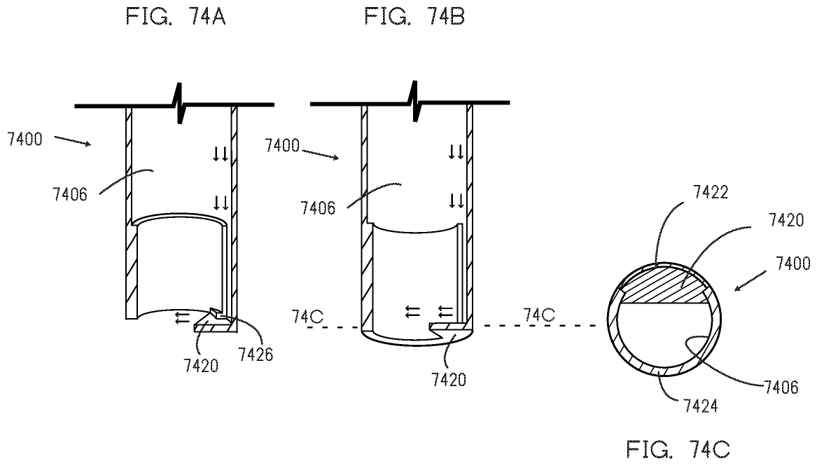

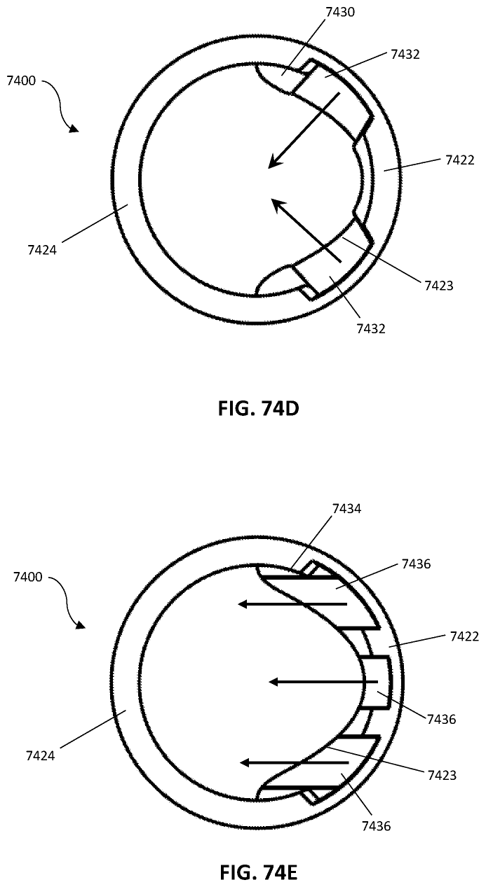

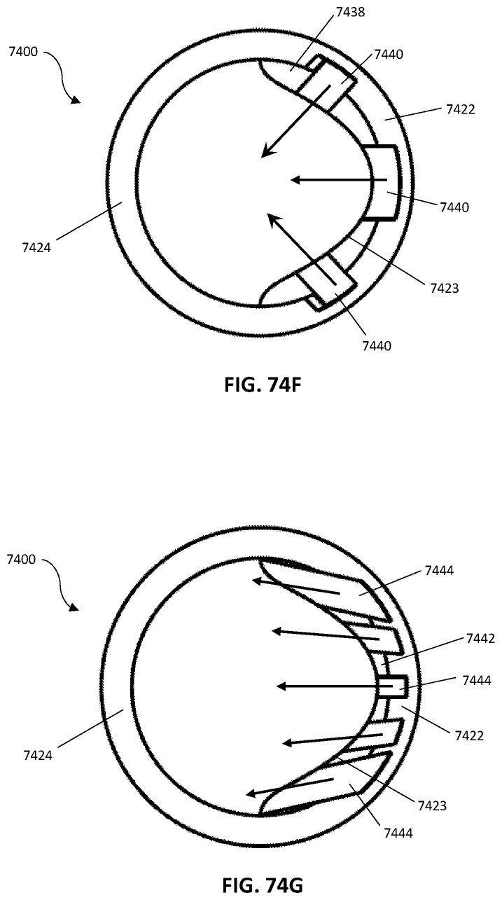

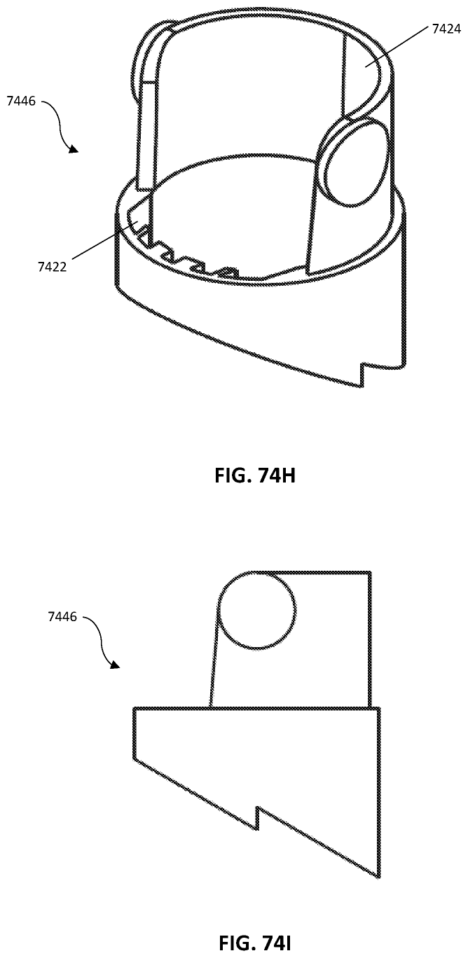

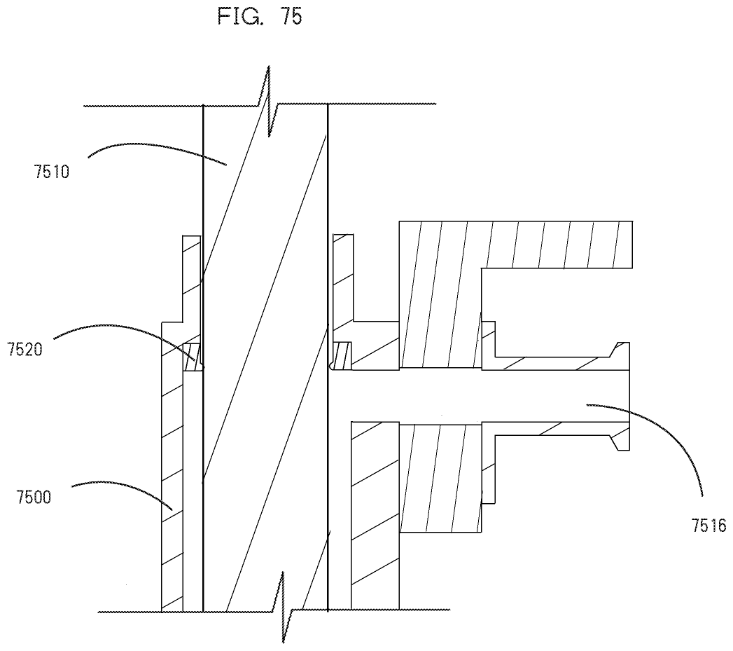

FIGS. 74A-74I illustrate embodiments of a medical instrument accessory 7400 including at least one deflection structure arranged to direct fluid flow transversely to a longitudinal axis defined by the accessory 7400. In the illustrated embodiments, the, or each, deflection structure is arranged to direct fluid flow relative to the distal end of the accessory 7400 and, as a result, relative to a distal end of a medical instrument received within the accessory 7400. In the embodiments of FIGS. 74A-74C, the deflection structure is in the form of a ledge 7420 extending partially across an opening defined in the distal end of the accessory 7400. It will be appreciated that in other embodiments, the deflection structure may be defined by a shelf, shoulder, bosses, or other structures which define one or more surfaces arranged to direct fluid flow. The medical instrument accessory 7400 has one or more sidewalls 7406 which define a first diameter portion 7424 and a second diameter portion 7422. Best shown in FIG. 74C, the first portion 7424 defines a first internal diameter dimensioned to be substantially the same as an external diameter of the medical instrument to form a close fit, and in some embodiments, friction fit, with the instrument. In the illustrated embodiments, the first portion 7424 extends away from the distal end of the accessory 7400 and partially along the shaft of the accessory 7400. In other embodiments, the first portion 7424 extends along the majority of the shaft of the accessory 7400. As the first portion 7424 is dimensioned to form a close fit to the instrument, little or no gas can flow past the instrument in this portion 7424 of the accessory 7400.

The second portion 7422 of the accessory 7400 defines a second internal diameter dimensioned to be greater than the first diameter. In the illustrated embodiments, the accessory 7400 is shaped to define the second diameter concentrically with the first diameter. It will be appreciated that, in other embodiments, the second diameter may be arranged about an axis which is axially offset from the axis of the first diameter—forming non-concentric diameters. The second portion 7422 can align with the position of the ledge 7420 with respect to the inner sidewall 7406. In some embodiments, the ledge can include various shapes other than tab-like protrusions, such as an arc or ramped region, for example. The second diameter portion 7422 can create a flow channel, indicated by the single-ended arrows. The flow channel directs flow longitudinally through the accessory 7400, past the instrument, and to the ledge 7420, where the fluid is directed transversely relative to the longitudinal axis of the accessory 7400, to flow at least partially in a non-parallel direction relative to the axis. In the illustrated embodiments, the fluid is directed substantially perpendicularly to the axis. For example, fluid is directed to allow flowing parallel to and across the distal end of an instrument, which can cause fluid flow parallel to a lens, arranged within the accessory 7400. It will be appreciated that in other embodiments, the fluid may be directed at other angles relative to the axis, for example, to cause fluid to be directed at, or away from, the distal end of the instrument. In some cases, the inner sidewall 7406 defines the second, larger diameter at the side of the accessory 7400 with the ledge 7420. This can allow the gas to flow down the accessory 7400 and be deflected by the ledge 7420 as it exits out of into the surgical cavity. FIG. 74C illustrates a cross section passing through line 74C-74C in FIG. 74B. FIG. 74C illustrates the first and second internal diameters. The accessory 7400 may include an abutment surface 7426 arranged to abut against the distal end of the instrument when received within the accessory 7400. In the illustrated embodiments, the abutment surface 7426 is defined by the first portion 7424 to be spaced axially from the ledge 7420. This allows positioning of the medical instrument in the accessory 7400 at a specific distance away from the ledge 7420 which can assist directing fluid, by the ledge 7420, across the end of the medical instrument. FIGS. 74A-74C show an embodiment of the accessory 7400 configured to receive a flat scope and lens. However, it should be understood that the accessory 7400 may have a distal end that is arranged transversely to the axis of accessory 7400, as described with reference to earlier embodiments, to accommodate a scope with an angled lens. It will be appreciated that the first portion 7424 and the second portion 7422 may be formed in an end cap which is securable, permanently or releasably, to a shaft which defines a constant internal diameter. In such embodiments, the shaft may define an internal diameter substantially equal to the internal diameter of the second portion 7422.

FIGS. 74D to 74I illustrate end views of alternative embodiments of the medical instrument accessory 7400 having differently configured distal end structures to allow directing fluid flow in one or more specific directions relative to the longitudinal axis of the accessory 7400, for example, by directing the air to form a plurality of directed streams. Each of the illustrated embodiments include a deflection structure extending from the second portion 7422 partially across the distal opening. Each deflection structure is arranged to direct fluid relative to the longitudinal axis of the accessory 7400, such as transversely to the axis, and/or perpendicularly or radially to the axis. Each deflection structure is arranged to receive fluid flowing through the accessory 7400, along its longitudinal axis, and deflect the fluid to flow transversely to the axis. Each structure may include curved or ramped portions arranged to enhance fluid flow transitioning from the axial direction to the transverse direction. In the illustrated embodiments, each deflection structure defines a curved, or compound curved, edge 7423 extending at least partially across the opening. In other embodiments, the deflection structure defines an at least partially straight edge extending at least partway across the opening, similar to the embodiment of FIGS. 74A-74C. In further embodiments, the deflection structure defines this edge to have a plurality of straight and/or curved regions, for example, extending along a faceted portion.

In FIG. 74D, the deflection structure, in this embodiment in the form of a shelf structure 7430, extends from the distal end to define a pair of deflection surfaces 7432. The surfaces 7432 are arranged to receive fluid flowing through the second portion 7424 of the accessory 7400, along the longitudinal axis of the accessory 7400 and past an instrument housed within the accessory 7400, and direct the received fluid across the axis. In the illustrated embodiment, the shelf structure 7430 is configured to be arranged adjacent a flat-ended medical instrument to direct the fluid substantially perpendicularly to the axis. The surfaces 7432 are arranged to direct fluid flow in two streams towards the axis, as indicated by the single-ended arrows. This allows directing the two streams of fluid towards an intersection region or point. For example, in the illustrated embodiment, the deflection surfaces 7432 are arranged to direct the streams of fluid radially to overlap at, or close to, the axis of the accessory 7400. As a result, the streams of fluid may intersect at, or close to, a centre of the distal end of the housed instrument, such as at the centre of a lens of scope. The directed streams may interact which can enhance clearing debris or fluid from the end of the instrument.

In FIG. 74E, the deflection structure, in this embodiment in the form of a shelf structure 7434, extends from the distal end to define a plurality of deflection surfaces 7436. The surfaces 7436 are arranged to receive fluid flowing through the second portion 7424 of the accessory 7400, along the longitudinal axis of the accessory 7400, and direct the received fluid to flow transversely to the axis, and in a plurality of parallel streams across the axis, as indicated by the single-ended arrows. In some embodiments, the surfaces 7436 are arranged to cause the parallel streams to be directed perpendicularly across the axis, for example, to allow flowing parallel to and across the distal end of a flat scope. In other embodiments, one or more of the surfaces 7436 may be arranged to cause the parallel streams to be directed at an angle across the axis, for example, to allow flowing parallel to across the distal end of an angled scope. The parallel streams may form a “blade” of fluid which can cause a shear effect to clear debris or fluid from the end of the instrument housed within the accessory 7400. In the illustrated embodiment, each of the deflection surfaces 7436 are planar and arranged co-planar with each other to allow forming the blade of fluid substantially across the distal end of the accessory 7400 and medical instrument. In other embodiments, the surfaces 7436 may be offset from each other on spaced planes to cause the streams of fluid to flow at spaced levels.

In FIG. 74F, the deflection structure, in this embodiment in the form of a shelf structure 7438, extends from the distal end to define deflection surfaces 7440. The surfaces 7440 are arranged to receive fluid flowing through the second portion 7424 of the accessory 7400 and direct the received in a plurality of streams towards the axis. The surfaces 7440 are arranged to direct the streams to intersect at, or near to, the axis, as indicated by the single-ended arrows, providing a combination of the approaches of the embodiments of FIGS. 74D and 74E.

In FIG. 74G, the deflection structure, in this embodiment in the form of a shelf structure 7442, extends from the distal end to define a plurality of deflection surfaces 7444. The surfaces 7444 are arranged to receive fluid flowing through the second portion 7424 of the accessory 7400 and direct the received fluid transversely to the axis in a plurality of diverging streams, as indicated by the single-ended arrows. The diverging streams may form a “blade” of fluid which can enhance causing a shear effect to clear debris or fluid from the end of the instrument housed within the accessory 7400. In the illustrated embodiment, the surfaces 7444 are arranged to cause the diverging streams to be directed at an angle across the axis, for example, to allow flowing parallel to across the distal end of an angled scope. This is more clearly illustrated in FIGS. 74H and 74I which show the deflection structure is formed in an end cap 7446 shaped to receive an angled scope. In other embodiments, the surfaces 7444 are arranged to cause the diverging streams to be directed parallel to the longitudinal axis of the accessory 7400. Referring to FIGS. 74H and 74I, the deflection structure is defined in the end cap 7446 which is securable to the shaft of the accessory 7400. Best shown in FIG. 74I, the end cap 7446 is shaped to receive an angled-end scope and arranged the deflection surfaces 7444 to direct flow at a complementary angle relative to the longitudinal axis of the accessory 7400 such that the fluid flows across the end of the scope. It will be appreciated that the embodiments of FIGS. 74D-74F may also be formed as end caps, each being securable to the shaft of the accessory 7400. This may allow providing a kit comprising the shaft and plurality of interchangeable, differently configured end caps to allow adjusting fluid flow relative to the end of the accessory 7400 to accommodate different medical instruments. It will be appreciated that the embodiments of FIGS. 74D-74G may be alternatively configured to define more or less deflection surfaces, and that the pluralities of deflection surfaces shown in these figures are exemplary.

FIG. 75 illustrates a medical instrument accessory 7500 with a seal element 7520 at the proximal end of the shaft of the accessory 7500 to prevent gas from exiting or leaking at the proximal end. As illustrated in FIG. 75, the medical instrument 7510 can be friction fit against the seal element 7520 to seal the proximal end of the lumen of the accessory 7500. The accessory 7500 can have a gas inlet 7516 that allows gas to be delivered directly to the lumen of the accessory 7500.

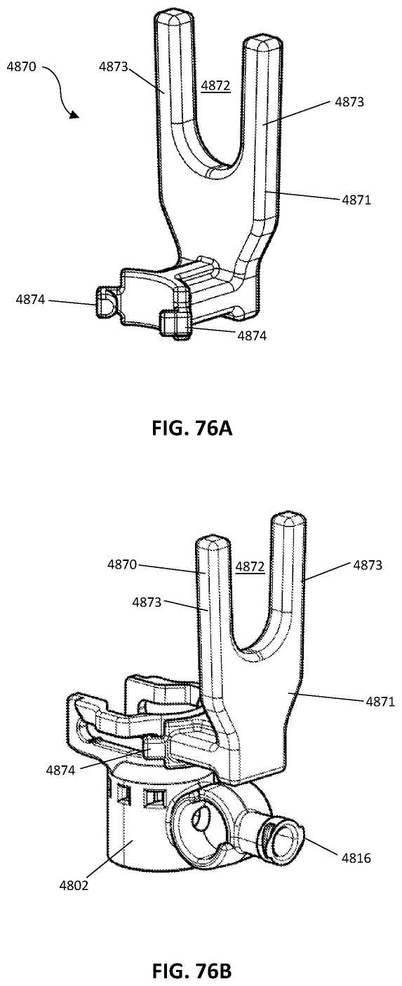

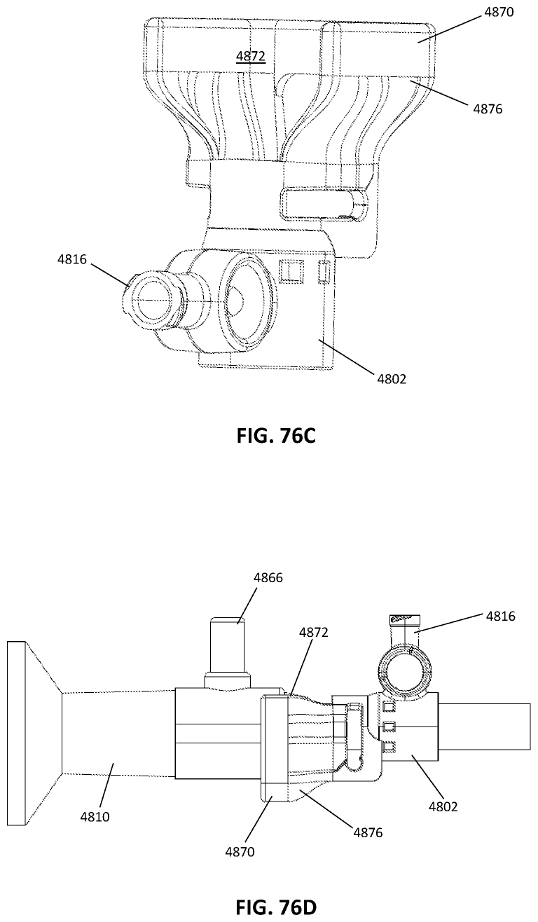

FIGS. 76A and 76B illustrate an alternative embodiment of the alignment feature 4870 discussed above, the feature 4870 comprising a body 4871 securable adjacent a proximal end of the accessory 4800. The body comprises a pair of elongate, spaced members 4873 which define the recess 4872. The members 4873 are configured to extend parallel to the longitudinal axis of the accessory 4800 such that the recess 4872 is an elongate, open ended slot. FIG. 76A shows a pair of tabs 4874 which are resiliently deformable to allow securing the alignment feature 4870 to the accessory 4800, such as forming a releasable ‘snap-fit’ connection. FIG. 76B shows the feature 4870 mounted adjacent to a proximal end of the accessory 4800. In some embodiments, the feature 4870 is mountable to the accessory 4800 such that the recess 4872 is aligned with the fluid inlet 4816. In this illustrated embodiment, the proximal end of the accessory 4800 is configured as an end cap 4802 releasably securable to a shaft. In other embodiments, two or more of the alignment feature 4800, the proximal end and the shaft are integrally formed.

FIGS. 76C and 76D illustrate a further alternative embodiment of the alignment feature 4870 including a shroud 4876 shaped and dimensioned to receive a section of the instrument 4810 to form a close fit. In this illustrated embodiment, the shroud defines a square or rectangular shaped cavity to allow mating to, and interlocking with, a square or rectangular section of the instrument 4810. Shown in FIG. 76D, the shroud 4876 defines the recess 4872 at a side arranged, in use, to be aligned with the connection port 4866 for connecting to a light cable. This configuration of the alignment feature 4870 is useful where the instrument 4810 defines a square or rectangular section as mating this section to the shroud 4876 can firmly inhibit relative rotation of the instrument 4810 and the accessory 4800. It will be appreciated that the shroud 4876 may be alternatively shaped to be complementary to the section of other instruments, such as defining opposed sides, for example, a hexagonal or oval/elliptical section instrument, or a circular-section instrument which has a protrusion which would abut against sides of the recess 4872, such as the light port 4866. It will also be appreciated that whilst the illustrated embodiment shows the shroud 4876 integrally formed with a proximal end cap 4802 of the accessory 4800, in other embodiments, the shroud 4876 is releasably connectable to the end cap 4802 and/or the accessory 4800. This may allow the shroud 4876 to be interchanged with the alignment feature 4870 embodiment shown in FIG. 76A.

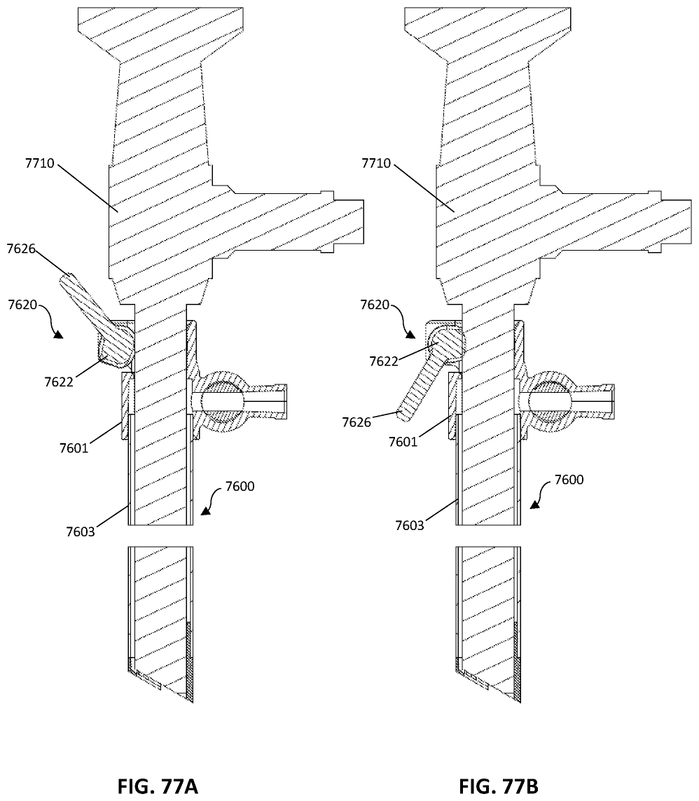

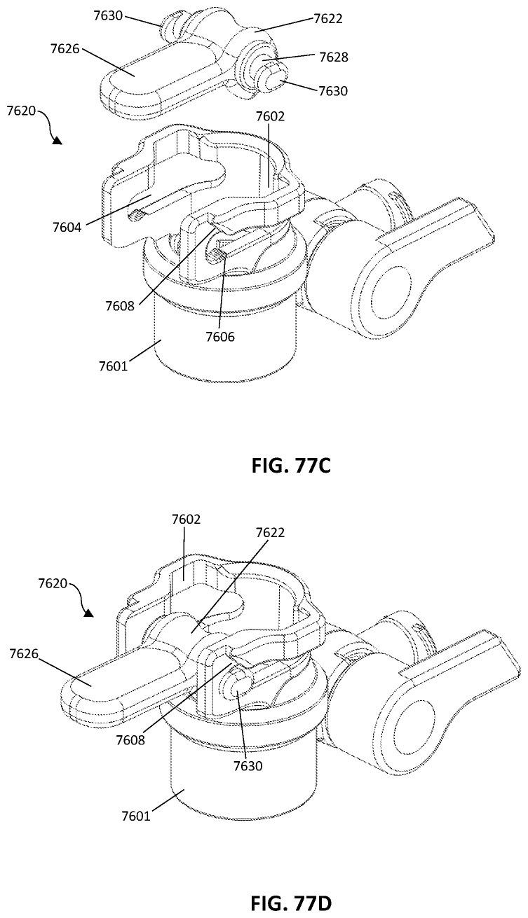

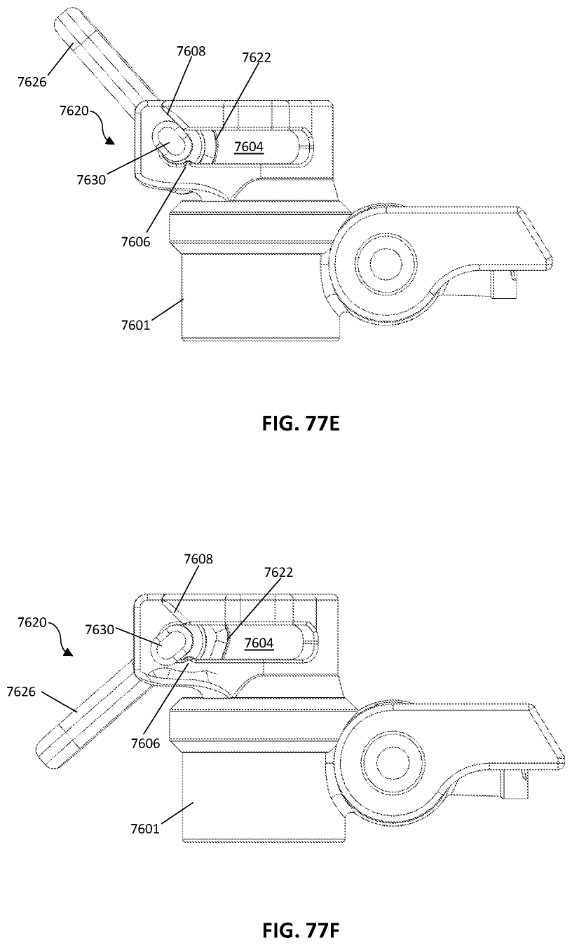

FIGS. 77A to 77F illustrate an embodiment of the locking mechanism 7620 comprising the cam 7622 and a proximal end cap 7601 of the accessory 7600. The end cap 7601 is securable to a shaft 7603 of the accessory 7600. It will be appreciated that in other embodiments, the shaft 7603 and end cap 7601 are integral. FIGS. 77A and 77B illustrate the arrangement of the cam 7622 in the open and locked positions relative to an instrument, being a scope 7710, arranged within the accessory 7600. In these figures, the cam 7622 is arranged about its rotational axis such that rotating the lever 7626 towards the accessory 7600, typically being operatively downwards, arranges the cam 7622 in the locked position. Shown in FIG. 77B, the cam 7622 is dimensioned such that when arranged in the locked position, the cam 7622 interferes with the instrument to cause a frictional engagement. The end cap 7601 defines an aperture dimensioned to receive a shaft of the instrument and may also include one or more seals arranged to extend at least partially around the aperture to allow inhibiting fluid from exiting the proximal end of the accessory 7600.

FIGS. 77C and 77D illustrate assembling the cam 7622 to the end cap 7601. As shown in these figures, the proximal end of the end cap 7601 defines a first, slot 7602 which intersects with a second, slot 7604 arranged to extend transversely to the first slot 7602. In the illustrated embodiment, the first slot 7602 extends parallel to the longitudinal axis of the accessory 7600, and the second slot 7604 extends perpendicularly to this axis. It will be appreciated that in other embodiments, the slots 7602, 7604 are alternatively arranged relative to each other. The cam 7622 includes a shaft 7628 extending from opposed sides, and a protrusion 7630 arranged at each end of the shaft 7628. The first slot 7602 is dimensioned to receive the shaft 7628 and the protrusions 7630, and the second slot 7604 is dimensioned to receive only the shaft 7628 so that the protrusions are arranged outside of the slot 7604. Securing the cam 7622 to the end cap 7601 involves passing the shaft 7628 and the protrusions 7630 through the first slot 7602 to a base of the slot 7602, and then moving the cam 7622 outwardly, away from the longitudinal axis of the accessory 7600 such that the shaft 7628 slides in the second slot 7604, with the protrusions 7630 arranged outside of the slot 7604, until the shaft 7628 rides over a detent feature 7606 arranged to retain the shaft 7628 at an end of the slot 7604. The shape of the protrusions 7630 and arrangement outside of the slot 7604 engages the cam 7622 with the accessory 7600. FIGS. 77E and 77F illustrate an abutment surface 7608 defined adjacent an end of the second slot 7604 and arranged to interfere with the protrusions 7630 to limit rotation of the cam 7622. Best shown in FIG. 77E, this inhibits further rotation of the cam 7622 to define the open position. FIG. 77F illustrates the cam 7622 arranged in the locked position.

### Terminology

Examples of medical gases delivery systems and associated components and methods have been described with reference to the figures. The figures show various systems and modules and connections between them. The various modules and systems can be combined in various configurations and connections between the various modules and systems can represent physical or logical links. The representations in the figures have been presented to clearly illustrate the principles and details regarding divisions of modules or systems have been provided for ease of description rather than attempting to delineate separate physical embodiments. The examples and figures are intended to illustrate and not to limit the scope of the inventions described herein. For example, the principles herein may be applied to delivery or venting of fluids from any desired site with respect to a patient's anatomy.

Examples described herein illustrate a concentric accessory used in combination with and supporting a medical instrument concentrically. In some cases, the accessory can be used to hold other medical instruments concentrically such as surgical tools. Additionally, as referred to herein the terms “concentric”, “concentrically”, and/or “substantially concentric” or any variations of these terms can also refer to minor axis offsets between the accessory and medical instrument.

Examples described herein refer to reducing fogging or condensation on the medical instrument. However, other obstructions to visualization or complications can be prevented or reduced. When reference is made herein to reducing fogging or condensation with the methods, procedures, and devices described herein, it can be understood that these methods, procedures, and devices can also reduce or prevent fogging, condensation, unwanted debris, and/or other field of view obstructions.

Although certain embodiments and examples are disclosed herein, inventive subject matter extends beyond the specifically disclosed embodiments to other alternative embodiments and/or uses, and to modifications and equivalents thereof. Thus, the scope of the claims or embodiments appended hereto is not limited by any of the particular embodiments described herein. For example, in any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain embodiments; however, the order of description should not be construed to imply that these operations are order dependent. Additionally, the structures described herein can be embodied as integrated components or as separate components. For purposes of comparing various embodiments, certain aspects and advantages of these embodiments are described. Not necessarily all such aspects or advantages are achieved by any particular embodiment. Thus, for example, various embodiments can be carried out in a manner that achieves or optimizes one advantage or group of advantages as taught herein without necessarily achieving other aspects or advantages as can also be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments. As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is otherwise understood with the context as used in general to convey that an item, term, etc. may be either X, Y or Z. Thus, such conjunctive language is not generally intended to imply that certain embodiments require at least one of X, at least one of Y and at least one of Z each to be present. As used herein, the words “about” or “approximately” can mean a value is within ±10%, within ±5%, or within ±1% of the stated value.

It should be emphasized that many variations and modifications may be made to the embodiments described herein, the elements of which are to be understood as being among other acceptable examples. All such modifications and variations are intended to be included herein within the scope of this disclosure and protected by the following claims. Further, nothing in the foregoing disclosure is intended to imply that any particular component, characteristic or process step is necessary or essential.

1. A medical instrument accessory for localizing insufflation or venting of fluid near a distal end of a medical instrument, the medical instrument accessory comprising:
a body mountable over at least a portion of a medical instrument shaft, the body having an inner lumen, proximal end and distal end, the distal end comprising an opening, wherein the distal end is configured to be arranged in use at or adjacent the distal end of the medical instrument; and
an outer wall of the medical instrument shaft and the inner lumen defining a fluid flow path, wherein fluid flows in and/or out of the fluid flow path at or adjacent the distal end of the medical instrument shaft.

2. The medical instrument accessory of claim 1, wherein the body is elongate.

3. The medical instrument accessory of claim 1 or 2, wherein the body is generally cylindrical.

4. The medical instrument accessory of any one of the preceding claims, wherein the fluid flow path is at least partly defined by an inner wall of the body and the outer wall of the medical instrument shaft.

5. The medical instrument accessory of any one of the preceding claims, wherein the body is configured to attach to the distal end of a cannula.

6. The medical instrument accessory of any one of the preceding claims, wherein the body is at least partially flexible and/or include an extendible element configured to attach to a distal end of the cannula.

7. The medical instrument accessory of any one of the preceding claims, wherein the body is movable between a retracted position and an extended position.

8. The medical instrument accessory of any one of the preceding claims, wherein the body is configured to attach to a proximal end of the medical instrument with an attachment.

9. The medical instrument accessory of the preceding claims, wherein the attachment is a sealing attachment.

10. The medical instrument accessory of the preceding claims, wherein the attachment is configured to create a fluid-tight seal.

11. The medical instrument accessory of the preceding claims, wherein the proximal end of the body is in fluid communication with a fluid source and/or vent.

12. The medical instrument accessory of the preceding claims, wherein the body has a first portion where the inner lumen has a first diameter substantially the same as the medical instrument outer wall diameter, and a second portion where the inner lumen has a second diameter, wherein the second diameter is greater than the medical instrument outer wall diameter, the body having at least one aperture in fluid communication with the fluid flow path.

13. The medical instrument accessory of claim 12, wherein the diameter of the lumen transitions from the first diameter to the second diameter.

14. The medical instrument accessory of claim 13, wherein the at least one aperture is located in the transition between the first and the second diameter.

15. The medical instrument accessory of any one of the preceding claims, wherein the medical instrument is a laparoscope and fluid is released from or introduced into the fluid flow path adjacent a lens of the laparoscope.

16. The medical instrument accessory of any one of the preceding claims, wherein the medical instrument is a laparoscope and fluid is released from or introduced into the fluid flow path parallel to a lens of the laparoscope.

17. The medical instrument accessory of any one of the preceding claims, wherein the body has a ring portion arranged at the distal end, the ring portion having at least one aperture on the surface of the ring.

18. The medical instrument accessory of claim 17, wherein the ring portion has an inner diameter that is smaller relative to the medical instrument diameter.

19. The medical instrument accessory of any one of the preceding claims, wherein the medical instrument is an electrocautery tool.

20. The medical instrument accessory of any one of the preceding claims, wherein the body has a length that extends substantially the length of the medical instrument such that the distal end is adjacent the distal end of the medical instrument.

21. A medical instrument accessory for localizing fluid flow around a distal end of a medical instrument, the accessory comprising:
a body configured to mount over at least a portion of a shaft of the medical instrument, the body comprising:
a lumen with an inner wall,
a proximal end,
an open distal end, and
at least one structure configured to, in use, position the medical instrument shaft in the lumen such that a fluid flow path is defined between the lumen inner wall and the medical instrument shaft and fluid can be directed into the open distal end or out from the open distal end and around an end of the medical instrument.

22. The medical instrument accessory of claim 21, wherein the at least one structure is on the inner wall of the accessory.

23. The medical instrument accessory of claim 21 or 22, wherein the body is configured to fit over at least a portion of the medical instrument shaft.

24. The medical instrument accessory of any one of claims 21 to 23, wherein the proximal end of the body is in fluid communication with a fluid source or vent.

25. The medical instrument accessory of any one of claims 21 to 24, wherein the at least one structure comprises a plurality of structures.

26. The medical instrument accessory of any one of claims 21 to 25, wherein the at least one structure holds the medical instrument shaft substantially concentrically in the lumen.

27. The medical instrument accessory of any one of claims 21 to 26, wherein the at least one structure comprises one or more ribs extending inwardly from the inner wall.

28. The medical instrument accessory of claim 27, wherein the one or more ribs extend substantially an entire length of the inner wall.

29. The medical instrument accessory of claim 27 or 28, wherein the one or more ribs are located about the open distal end.

30. The medical instrument accessory of any one of claims 27 to 29, wherein the one or more ribs are located adjacent the proximal end.

31. The medical instrument accessory of any one of claims 27 to 30, wherein the one or more ribs are located adjacent the proximal and distal open end.

32. The medical instrument accessory of any one of claims 21 to 31, wherein the at least one structure comprises one or more protrusions extending inwardly from the inner wall of the lumen.

33. The medical instrument accessory of claim 32, wherein the one or more protrusions are located in one or more of the proximal end, distal end or intermediate along the length of the lumen.

34. The medical instrument accessory of any one of claims 27 to 33, wherein the one or more ribs or one or more protrusions are spaced substantially uniformly around a diameter of the lumen.

35. The medical instrument accessory of any one of claims 27 to 33, wherein the one or more ribs or one or more protrusions are spaced non-uniformly, defining fluid flow paths of different sizes.

36. The medical instrument accessory of any one of claims 21 to 35, wherein the at least one structure comprises one or more fins extending inwardly from the lumen inner wall.

37. The medical instrument accessory of claim 36, wherein the one or more fins are arranged in a substantially spiral configuration.

38. The medical instrument accessory of any one of claims 21 to 37, wherein the at least one structure comprises one or more flexible members extending from the open distal end.

39. The medical instrument accessory of any one of claims 21 to 38, wherein the at least one structure comprises a movable tip located at the distal open end, the movable tip having a flexible portion and a solid edge with one or more protrusions extending radially inwardly.

40. The medical instrument accessory of claim 39, wherein the solid edge is laterally movable and is substantially parallel with the open distal end.

41. The medical instrument accessory of claim 39 or 40, wherein the solid edge is configured to engage with an end of the medical instrument.

42. The medical instrument accessory of any one of claims 21 to 41, wherein the lumen has a cross-sectional shape of a different shape than the medical instrument shaft.

43. The medical instrument accessory of any one of claims 21 to 42, wherein the cross-sectional shape of the lumen is substantially oval shaped.

44. The medical instrument accessory of any one of claims 21 to 43, wherein the at least one structure comprises one or more channels disposed in the inner wall of the lumen.

45. The medical instrument accessory of claim 44, wherein the one or more channels extend substantially an entire length of the lumen.

46. A medical instrument accessory for directing fluid flow around and/or across a distal end of a medical instrument, the accessory comprising:
a body configured to mount over at least a portion of a shaft of the medical instrument, the body comprising:
a lumen with an inner wall;
a proximal end;
an open distal end; and
a stopping portion at or adjacent the open distal end.

47. The medical instrument accessory of claim 46, wherein the stopping portion is configured to in use, locate an end of the medical instrument shaft a predetermined distance away from the open distal end of the body.

48. The medical instrument accessory of claim 46 or 47, further comprising one or more protrusions disposed on, or adjacent, the inner wall of the lumen.

49. The medical instrument accessory of claim 48, comprising a plurality of the protrusions arranged substantially concentrically around the inner wall of the lumen.

50. The medical instrument accessory of any one of claims 46 to 49, wherein the body defines a longitudinal axis between the ends, and further comprises a deflection structure extending partially across the open distal end to allow receiving fluid flowing through the lumen and deflecting the fluid to flow transversely to the longitudinal axis.

51. The medical instrument accessory of claim 50, wherein the deflection structure extends radially inward from an edge of the open distal end.

52. The medical instrument accessory of claim 50 or 51, wherein the deflection structure includes a plurality of deflection surfaces arranged to direct fluid in a respective plurality of streams across the longitudinal axis.

53. The medical instrument accessory of any one of claims 50 to 52, wherein the deflection structure is configured as a ledge.

54. The medical instrument accessory of claim 53, wherein the ledge is configured in a ring extending substantially perpendicular from the edge of the open distal end.

55. The medical instrument accessory of claim 53 or 54, wherein the ledge comprises a surface area that is a segment of a circle.

56. The medical instrument accessory of any one of claims 46 to 55, wherein the stopping portion is arranged in the lumen to be spaced axially from the ledge.

57. The medical instrument accessory of any one of claims 46 to 56, further comprising a protuberance extending longitudinally from the open distal end.

58. The medical instrument accessory of claim 57, wherein the protuberance extends from an edge of the open distal end to partially surround the opening.

59. The medical instrument accessory of claim 57 or 58, wherein the protuberance is configured as a flange.

60. The medical instrument accessory of any one of claims 57 to 59, wherein the flange defines a free end, and the ledge extends radially inwards from the free end of the flange.

61. The medical instrument accessory of any one of claims 46 to 60, wherein the body includes a second lumen configured to channel insufflation gas out of the accessory to allow directing the insufflation gas into a surgical cavity.

62. The medical instrument accessory of any one of claims 46 to 61, wherein the body includes a venting lumen configured to vent fluid from adjacent the accessory to allow venting fluid from a surgical cavity.

63. The medical instrument accessory of claim 62, wherein the venting lumen has an inlet arranged in the body.

64. The medical instrument accessory of any one of claims 46 to 63, wherein the open distal end is angled relative to a longitudinal axis of the body.

65. A medical instrument accessory for heating a medical instrument, comprising:
a body configured to mount over at least a portion of the medical instrument, the body including a heating device;
wherein the heating device, in use, directly or indirectly heats the medical instrument.

66. The medical instrument accessory of claim 60, wherein the body has a lumen with an inner wall, the inner wall configured to contact a surface of the medical instrument.

67. The medical instrument accessory of claim 60, wherein the body has a lumen with an inner wall, the inner wall configured in use to be spaced apart from a surface of the medical instrument.

68. The medical instrument accessory of any one of claims 60-62, wherein the body has a length less than an entire length of a shaft of the medical instrument.

69. The medical instrument accessory of any one of claims 60 to 63, wherein the heating device comprises one or more selected from the group consisting of: heating coils, resistive material, flexible PCB, chemical heating, insulated material, and vaporization.

70. The medical instrument accessory of any one of claims 60 to 64, wherein the heating device is powered by one or more of an external unit, an associated cannula, a battery, a tubeset, a tube, and a wireless power transfer.

71. The medical instrument accessory of any one of claims 60 to 65, wherein the heating device provides heating substantially along an entire length of a shaft of the medical instrument.

72. The medical instrument accessory of any one of claims 60 to 66, wherein the heating device is configured to provide graduated heat along a shaft of the medical instrument.

73. The medical instrument accessory of any one of claims 60 to 67, wherein the heating device is configured to provide heat localized to a portion of a shaft of the medical instrument.

74. A medical instrument accessory for localizing fluid flow around a distal end of a medical instrument, the accessory comprising:
a body configured to mount over at least a portion of a shaft of the medical instrument,
the body having at least one structure configured to in use, position the medical instrument shaft in a cannula lumen such that a fluid flow path is defined between a wall of the cannula lumen and the medical instrument shaft.

75. The medical instrument accessory of claim 69, wherein the fluid flow path directs fluid around or across a distal end of the medical instrument.

76. The medical instrument accessory of claim 69 or 70, wherein the at least one structure comprises a plurality of structures.

77. The medical instrument accessory of any one of claims 69 to 71, wherein the at least one structure is configured to, in use, lie on or reside adjacent an outer surface of the medical instrument.

78. A medical instrument for use in laparoscopic surgical procedures, the medical instrument comprising:
a shaft configured to direct fluid flow over or adjacent to a distal end of the shaft.

79. The medical instrument of claim 73, wherein the shaft has a lumen to direct fluid flow through the shaft and out the distal end of the shaft.

80. The medical instrument of claim 74, wherein the lumen is concentric with respect to the shaft.

81. The medical instrument of any one of claims 73-75, wherein the medical instrument comprises a ledge extending inwardly from an end of the shaft and a flange directing fluid flow from the concentric lumen across the distal end of shaft.

82. The medical instrument of claim 76, wherein the ledge is ring shaped.

83. The medical instrument of any one of claims 74-77, wherein the lumen is offset from a center of the shaft.

84. The medical instrument of claim 76, wherein the ledge extends inwardly from the edge of a distal end of the shaft and the flange is located adjacent to a lumen opening.

85. The medical instrument of any one of claims 73 to 79, wherein a surface of the shaft has one or more protrusions extending radially outwards, the one or more protrusions configured, in use, to contact an inner wall of a cannula.

86. The medical instrument of claim 80, wherein the cannula inner wall and one or more protrusions define a fluid flow path.

87. The medical instrument of claim 80 or 81, wherein the one or more protrusions are ribs, extending at least part of the length of the shaft.

88. The medical instrument of claim 82, wherein the ribs extend substantially an entire length of the shaft.

89. The medical instrument of any one of claims 80 to 83, wherein the one or more protrusions are arranged substantially uniformly around a circumference of the shaft.

90. The medical instrument of any one of claims 80 to 83, wherein the one or more protrusions are arranged non-uniformly around a circumference of shaft.

91. The medical instrument of any one of claims 80 to 83, wherein the one or more protrusions are different sizes and around a circumference of shaft, wherein the different sizes of the one or more protrusions are configured to create varied gas path sizes.

92. The medical instrument of any one of claims 80 to 86, wherein the one or more protrusions comprises a spiral fin.

93. The medical instrument of any one of claims 73 to 87, wherein the shaft has a cross-sectional shape different from a cross-sectional shape of the cannula.

94. The medical instrument of any one of claims 73 to 88, wherein the shaft of the medical instrument comprises a heating device.

95. A medical instrument accessory for localizing insufflation or venting of fluid from a distal end of a medical instrument having a shaft, the medical instrument accessory including:
a body configured to be mounted to the medical instrument, the body defining an inner lumen dimensioned to receive at least a portion of the shaft, a proximal end, a distal end defining an opening, and a longitudinal axis between the ends,
the distal end being configured to be arranged, in use, at, or adjacent, the distal end of the medical instrument, and
the inner lumen being shaped to define a fluid flow path, wherein, in use, fluid flows in and/or out of the fluid flow path through the opening.

96. The medical instrument accessory of claim 95, including at least one deflection structure arranged to receive fluid flowing through the inner lumen and direct the received fluid to flow transversely to the longitudinal axis.

97. The medical instrument accessory of claim 96, wherein the at least one deflection structure is configured such that, in use, the, or each, deflection structure directs the received fluid to flow substantially across the distal end of the medical instrument.

98. The medical instrument accessory of claim 97, wherein the at least one deflection structure is configured to, in use, direct the received fluid to flow substantially parallel to the distal end of the medical instrument.

99. The medical instrument accessory of any one of claims 96 to 98, wherein the at least deflection structure is arranged to direct the received fluid to flow substantially perpendicularly to the longitudinal axis.

100. The medical instrument accessory of any one of claims 96 to 99, wherein the at least one deflection structure is arranged to direct the received fluid in a plurality of separate streams.

101. The medical instrument accessory of claim 100, wherein the at least one deflection structure is arranged such that at least two of the streams are directed to intersect with each other.

102. The medical instrument accessory of claim 101, wherein the at least one deflection structure is arranged such that each of the at least two of the streams are directed radially towards the longitudinal axis.

103. The medical instrument accessory of claim 100, wherein the at least one deflection structure is arranged such that at least two of the streams are directed to be parallel to each other.

104. The medical instrument accessory of claim 100, wherein the at least one deflection structure is arranged such that at least two of the streams are directed to diverge away from each other.

105. The medical instrument accessory of any one of claims 100 to 104, wherein the at least one deflection structure is arranged such that the plurality of streams are directed across two or more planes spaced axially apart from each other.

106. The medical instrument accessory of any one of claims 96 to 105, wherein the, or each, deflection structure is arranged to extend from one half of the inner lumen.

107. The medical instrument accessory of any one of claims 96 to 105, wherein the, or each, deflection structure is arranged to cover equal to, or less than, half of the opening defined by the distal end.

108. The medical instrument accessory of any one of claims 96 to 107, wherein the inner lumen defines two portions, a first portion defining a first diameter, and a second portion defining a second diameter which is greater than the first diameter, and wherein the, or each, deflection structure extends from the second portion partially across the opening.

109. The medical instrument accessory of claim 108, wherein the first diameter is dimensioned to be substantially equivalent to an external diameter of the medical instrument such that, in use, the first portion forms a close fit with the medical instrument.

110. The medical instrument accessory of any one of claims 96 to 109, wherein the body comprises a shaft and an end cap releasably securable to the shaft, and wherein the at least one deflection structure is defined by the end cap.

111. The medical instrument accessory of any one of claims 95 to 110 including an alignment feature defining a recess shaped to at least partially receive a portion of the medical instrument, the recess arranged to inhibit relative rotation of the medical instrument and the accessory.

112. The medical instrument accessory of claim 111, wherein the alignment feature is arranged at, or adjacent to, the proximal end of the body.

113. The medical instrument accessory of claim 111 or 112, wherein the recess is configured to be an open-ended slot configured to extend along the longitudinal axis.

114. The medical instrument accessory of claim 113, wherein the recess is defined by a pair of spaced elongate members.

115. The medical instrument accessory of claim 111 or 112, wherein the recess is defined by a shroud shaped to be complementary to, and at least partially surround, the portion of the medical instrument.

116. The medical instrument accessory of any one of claims 111 to 115, wherein the body comprises a shaft and an end cap releasably securable to the shaft, and wherein the alignment feature extends from the end cap.

117. The medical instrument accessory of claim 116, wherein the alignment feature is releasably securable to the end cap.

118. The medical instrument accessory of any one of claims 95 to 117 including a locking mechanism operable to retain the medical instrument in the accessory.

119. The medical instrument accessory of claim 118, wherein the locking mechanism includes a cam rotatable about an axis between an open position and a locked position, wherein, in the locked position in use, the cam is arranged to interfere with the medical instrument.

120. The medical instrument accessory of claim 118 or 119 wherein the locking mechanism is arranged at, or adjacent to, the proximal end of the body.

121. The medical instrument accessory of any one of claims 118 to 120, wherein the body defines a first slot and a second slot extending perpendicularly to the first slot and intersecting with the first slot, and wherein the cam includes shaft, and each slot is dimensioned to receive the shaft.

122. The medical instrument accessory of claim 121, wherein the cam includes a protrusion at each end of the shaft, and wherein the first slot is dimensioned to receive the shaft and the protrusions, and the second slot is dimensioned to receive only the shaft such that the protrusions are arranged outside of the second slot to engage the cam with the body.
